SEQUENCE LISTING (18 sequences)

SEQ ID NO: 1 - DNA, 1111 bp, Mus musculus
  source 1..1111: mol_type = genomic DNA
agcgtcaaagaagcaggtgagaaaggggccacctatactcctgtgaattagacacatgcacaaaagtgaaaaagaatttgtgtctataatcagtgagtgcactgtaatctcttcattatatccgctgacttttcagagttccaaaactttactgctatgcaaaccacattaacttcaatccttctgcccatttggtgtaagcatgcatggaccaggttgtggattctgacagccaaggaacctgggtgcttcaacagggaagctgttctcatagccatcgtggtgtccctgtgagacaaccattaagagtcatggctctctggacttgagaaagggaaagaaaacaggagggtaagattcgtggggaaaatcctgtgtccttcccttccaccaaggtgtctctgttgctccataaataaaacgtcccaccgctttgagtgagagctataaaggcagcagaaaaactgtccccaggcattctggtcgggcagaactccggagcccaaacaggtgagtttatcacagaagagatgcagtcggctgcaccctgcccgctctgtcgtaactctctagggaaggtactggtcaggcgccgcgggcggggtgggcaggacagacaccagaaaaagcgagacaagaggaagctaagggtcagaccgggaaactcgcacctgtccttaaatgggactgccctgctgcagcccagagtgtgcgtggagctgtgtctccgatcgagtgaggggagtaccggaccctttccctgtcgcacaggccccagtccgattccggggagatttcaggacgcggacagcgccctgaattgcccagggaggccacccagcccaacacccaagccctcaaagtgaggctccctcccaggacagcggggattcccgggtatctggtacgggagactgcagtcctcgggtaccgtcctgcaggtggccggccgcagggacagtttgttgccagcgccgaggactgcgggacgcggaccggctttccgaagtcgctgccacaggtggcacccgaggctgctcccaggtgcccgccgcgggccgtttgctcgctccgcgtttccgaacgtcatccctcgtcgcatcccctccag

SEQ ID NO: 2 - DNA, 390 bp, Mus musculus
  source 1..390: mol_type = genomic DNA
taagtagaatacgtggttagaaaatccagcgctaagtagggtgacaagcaaggaactgttaaagccagtggagttgggttttttttttttttccaattttttcctcttcttttatttcagacaataattgctactgccatttcctatggggccaacttggaaaagaactacaatgctggcattgttaagtccatcccaagtgggtgagtgtggccttccttccagatggcactaataaactagggcagcactttgtttaaatggagctttttattactggttttgctttactatttattgatagtcttacttgcatgtgattttggggaaatgtcatgtgcactacacatcagacaaacagcgcatgtggggaccaagttcaggccattt

SEQ ID NO: 3 - DNA, 317 bp, Mus musculus
  source 1..317: mol_type = genomic DNA
cagtcaggacttctgtgagtccacggcctttgtctgtaagctctccgctgactcagggaaggaagtggtgagaaaggctggaaggtggacaggagccagataatgaagggcctcacacactgcattaaggagcttggattttaatctcatctggtaagagataaaggcaaagtggtggcttacattacctggctaatccaatcagctgacctaggtacctagaaatagattcagaggaaattaaagtagagatgaatgaagcttttgtttttgtttcttttttttttctcccaaggagagagtaagagatgaaagaa

SEQ ID NO: 4 - protein, 780 aa, Homo sapiens
MAAPGGRSEPPQLPEYSCSYMVSRPVYSELAFQQQHERRLQERKTLRESLAKCCSCSRKRAFGVLKTLVPILEWLPKYRVKEWLLSDVISGVSTGLVATLQGMAYALLAAVPVGYGLYSAFFPILTYFIFGTSRHISVGPFPVVSLMVGSVVLSMAPDEHFLVSSSNGTVLNTTMIDTAARDTARVLIASALTLLVGIIQLIFGGLQIGFIVRYLADPLVGGFTTAAAFQVLVSQLKIVLNVSTKNYNGVLSIIYTLVEIFQNIGDTNLADFTAGLLTIVVCMAVKELNDRFRHKIPVPIPIEVIVTIIATAISYGANLEKNYNAGIVKSIPRGFLPPELPPVSLFSEMLAASFSIAVVAYAIAVSVGKVYATKYDYTIDGNQEFIAFGISNIFSGFFSCFVATTALSRTAVQESTGGKTQVAGIISAAIVMIAILALGKLLEPLQKSVLAAVVIANLKGMFMQLCDIPRLWRQNKIDAVIWVFTCIVSIILGLDLGLLAGLIFGLLTVVLRVQFPSWNGLGSIPSTDIYKSTKNYKNIEEPQGVKILRFSSPIFYGNVDGFKKCIKSTVGFDAIRVYNKRLKALRKIQKLIKSGQLRATKNGIISDAVSTNNAFEPDEDIEDLEELDIPTKEIEIQVDWNSELPVKVNVPKVPIHSLVLDCGAISFLDVVGVRSLRVIVKEFQRIDVNVYFASLQDYVIEKLEQCGFFDDNIRKDTFFLTVHDAILYLQNQVKSQEGQGSILETITLIQDCKDTLELIETELTEEELDVQDEAMRTLAS

SEQ ID NO: 5 - protein, 780 aa, Mus musculus
MAARGGRSEPPQLAEYSCSYTVSRPVYSELAFQQQRERRLPERRTLRDSLARSCSCSRKRAFGVVKTLLPILDWLPKYRVKEWLLSDIISGVSTGLVGTLQGMAYALLAAVPVQFGLYSAFFPILTYFVFGTSRHISVGPFPVVSLMVGSVVLSMAPDDHFLVPSGNGSALNSTTLDTGTRDAARVLLASTLTLLVGIIQLVFGGLQIGFIVRYLADPLVGGFTTAAAFQVLVSQLKIVLNVSTKNYNGILSIIYTLIEIFQNIGDTNIADFIAGLLTIIVCMAVKELNDRFKHRIPVPIPIEVIVTIIATAISYGANLEKNYNAGIVKSIPSGFLPPVLPSVGLFSDMLAASFSIAVVAYAIAVSVGKVYATKHDYVIDGNQEFIAFGISNVFSGFFSCFVATTALSRTAVQESTGGKTQVAGLISAVIVMVAIVALGRLLEPLQKSVLAAVVIANLKGMFMQVCDVPRLWKQNKTDAVIWVFTCIMSIILGLDLGLLAGLLFALLTVVLRVQFPSWNGLGSVPSTDIYKSITHYKNLEEPEGVKILRFSSPIFYGNVDGFKKCINSTVGFDAIRVYNKRLKALRRIQKLIKKGQLRATKNGIISDIGSSNNAFEPDEDVEEPEELNIPTKEIEIQVDWNSELPVKVNVPKVPIHSLVLDCGAVSFLDVVGVRSLRMIVKEFQRIDVNVYFALLQDDVLEKMEQCGFFDDNIRKDRFFLTVHDAILHLQNQVKSREGQDSLLETVARIRDCKDPLDLMEAEMNAEELDVQDEAMRRLAS

SEQ ID NO: 6 - DNA, 2340 bp, Homo sapiens
  source 1..2340: mol_type = other DNA
atggcagcgccaggcggcaggtcggagccgccgcagctccccgagtacagctgcagctacatggtgtcgcggccggtctacagcgagctcgctttccagcaacagcacgagcggcgcctgcaggagcgcaagacgctgcgggagagcctggccaagtgctgcagttgttcaagaaagagagcctttggtgtgctaaagactcttgtgcccatcttggagtggctccccaaataccgagtcaaggaatggctgcttagtgacgtcatttcgggagttagtactgggctagtggccacgctgcaagggatggcatatgccctactagctgcagttcctgtcggatatggtctctactctgcttttttccctatcctgacatactttatctttggaacatcaagacatatctcagttggaccttttccagtggtgagtttaatggtgggatctgttgttctgagcatggcccccgacgaacactttctcgtatccagcagcaatggaactgtattaaatactactatgatagacactgcagctagagatacagctagagtcctgattgccagtgccctgactctgctggttggaattatacagttgatatttggtggcttgcagattggattcatagtgaggtacttggcagatcctttggttggtggcttcacaacagctgctgccttccaagtgctggtctcacagctaaagattgtcctcaatgtttcaaccaaaaactacaatggagttctctctattatctatacgctggttgagatttttcaaaatattggtgataccaatcttgctgatttcactgctggattgctcaccattgtcgtctgtatggcagttaaggaattaaatgatcggtttagacacaaaatcccagtccctattcctatagaagtaattgtgacgataattgctactgccatttcatatggagccaacctggaaaaaaattacaatgctggcattgttaaatccatcccaagggggtttttgcctcctgaacttccacctgtgagcttgttctcggagatgctggctgcatcattttccatcgctgtggtggcttatgctattgcagtgtcagtaggaaaagtatatgccaccaagtatgattacaccatcgatgggaaccaggaattcattgcctttgggatcagcaacatcttctcaggattcttctcttgttttgtggccaccactgctctttcccgcacggccgtccaggagagcactggaggaaagacacaggttgctggcatcatctctgctgcgattgtgatgatcgccattcttgccctggggaagcttctggaacccttgcagaagtcggtcttggcagctgttgtaattgccaacctgaaagggatgtttatgcagctgtgtgacattcctcgtctgtggagacagaataagattgatgctgttatctgggtgtttacgtgtatagtgtccatcattctggggctggatctcggtttactagctggccttatatttggactgttgactgtggtcctgagagttcagtttccttcttggaatggccttggaagcatccctagcacagatatctacaaaagtaccaagaattacaaaaacattgaagaacctcaaggagtgaagattcttagattttccagtcctattttctatggcaatgtcgatggttttaaaaaatgtatcaagtccacagttggatttgatgccattagagtatataataagaggctgaaagcgctgaggaaaatacagaaactaataaaaagtggacaattaagagcaacaaagaatggcatcataagtgatgctgtttcaacaaataatgcttttgagcctgatgaggatattgaagatctggaggaacttgatatcccaaccaaggaaatagagattcaagtggattggaactctgagcttccagtcaaagtgaacgttcccaaagtgccaatccatagccttgtgcttgactgtggagctatatctttcctggacgttgttggagtgagatcactgcgggtgattgtcaaagaattccaaagaattgatgtgaatgtgtattttgcatcacttcaagattatgtgatagaaaagctggagcaatgcgggttctttgacgacaacattagaaaggacacattctttttgacggtccatgatgctatactctatctacagaaccaagtgaaatctcaagagggtcaaggttccattttagaaacgatcactctcattcaggattgtaaagatacccttgaattaatagaaacagagctgacggaagaagaacttgatgtccaggatgaggctatgcgtacacttgcatcc

SEQ ID NO: 7 - DNA, 2340 bp, Mus musculus
  source 1..2340: mol_type = other DNA
atggcagcgcggggcggcaggtcggagccgccgcagctcgccgagtacagctgcagttacacggtgtcgcggccggtgtacagcgagctcgccttccagcagcagcgcgagcggcgcctgcctgagcgcaggacgctgcgggacagcctggcgcggagctgcagttgctcaagaaagagagcctttggtgtggtaaagactctcctgcccattctggactggctcccaaaataccgagtcaaggaatggctcctcagtgacatcatctccggagttagcactgggctggtgggtaccctgcaagggatggcttatgccctgctggcagcagtacctgttcagttcggtctctactctgcctttttccctatcctgacgtattttgtgttcggaacatcaagacacatctcagttggccctttccccgtggtcagtttaatggtgggatctgttgttctgagcatggctccagatgaccactttcttgtgcccagcggtaacggaagtgcattgaactcgaccacgttagacactggaaccagagatgcggcccgagtgttgcttgcaagcacactcactcttctagttggaatcatacagctggtgtttggaggtttgcagattggattcatagtgaggtacttggcagaccccttggttggcggattcacaaccgctgcagccttccaagtactggtctcacagctaaagatcgtgctcaatgtttcaaccaaaaactacaacggcatcctctccattatctacacactaattgagatttttcaaaatatcggtgacaccaatattgccgatttcatcgctgggctgctgaccatcatcgtctgtatggctgttaaggaactaaatgatcgatttaaacacagaatcccggtgcccattcctatagaagtgattgtgacaataattgctactgccatttcctatggggccaacttggaaaagaactacaatgctggcattgttaagtccatcccaagtgggttcttgcctcctgtcctgccatctgtgggcctgttttcggacatgttggctgcatccttttccattgctgtggtggcttacgctattgcagtgtctgtaggaaaagtctacgccaccaagcatgactatgtcatcgatgggaaccaggaattcattgcctttgggataagcaacgtcttctctggatttttctcctgttttgtggctaccactgctctgtctcgaacggctgtccaggagagcaccggggggaagacacaggtggctggcctcatctcagctgtgattgtgatggttgccatcgttgccttggggaggcttctggaacccttgcagaagtcagtcttggcggccgttgtcattgccaacctgaaagggatgtttatgcaggtgtgtgacgttcctcgtctgtggaagcagaataagactgatgctgttatctgggtgtttacatgcataatgtccatcattctggggctggacctcggcttgctagctggccttttatttgcactactgactgtggtcctgagagttcagttcccttcatggaatggccttggaagtgtccccagcacagacatctacaaaagcatcacacattataaaaaccttgaagagcctgaaggggtgaagatcctgagattttccagtcccattttttacggcaatgtcgatggttttaaaaaatgtatcaattcaacggttggatttgatgccattagagtatataataagaggctgaaagcactgagaagaatacagaaactcatcaaaaaaggacaactcagggcaaccaagaacgggatcataagtgatattggctcatcaaataatgccttcgagcctgatgaagatgtggaagagccagaggaacttaatatcccaaccaaagaaattgagattcaagtggactggaactccgaactcccggtgaaagtgaatgtcccaaaggtgccaatccacagcctggtgctggattgcggagctgtatccttcctggatgtggtaggagtgaggtcattgcgaatgattgtcaaagaatttcagagaattgatgtgaatgtgtactttgctctgcttcaagatgatgtgttagaaaagatggagcagtgtgggttctttgatgacaacattagaaaggacagattctttctgacggttcatgatgcaatcctccatctgcagaaccaggtcaaatccagagaaggccaggattccctgctagagacggtcgctcgcattcgggactgtaaagaccctcttgatctgatggaggcagagatgaatgcagaagagctcgatgttcaggatgaggccatgcgtagacttgcttcc

SEQ ID NO: 8 - protein, 354 aa, Homo sapiens
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENSKTSPRSHRSDGEFSPHSHYSDSDEAS

SEQ ID NO: 9 - DNA, 1062 bp, Homo sapiens
  source 1..1062: mol_type = other DNA
atgtcccgcctgctgcatgcagaagagtgggctgaagtgaaggagttgggagaccaccatcgccagccccagccgcatcatctcccgcaaccgccgccgccgccgcagccacctgcaactttgcaggcgagagagcatcccgtctacccgcctgagctgtccctcctggacagcaccgacccacgcgcctggctggctcccactttgcagggcatctgcacggcacgcgccgcccagtatttgctacattccccggagctgggtgcctcagaggccgctgcgccccgggacgaggtggacggccggggggagctggtaaggaggagcagcggcggtgccagcagcagcaagagccccgggccggtgaaagtgcgggaacagctgtgcaagctgaaaggcggggtggtggtagacgagctgggctgcagccgccaacgggccccttccagcaaacaggtgaatggggtgcagaagcagagacggctagcagccaacgccagggagcggcgcaggatgcatgggctgaaccacgccttcgaccagctgcgcaatgttatcccgtcgttcaacaacgacaagaagctgtccaaatatgagaccctgcagatggcccaaatctacatcaacgccttgtccgagctgctacaaacgcccagcggaggggaacagccaccgccgcctccagcctcctgcaaaagcgaccaccaccaccttcgcaccgcggcctcctatgaagggggcgcgggcaacgcgaccgcagctggggctcagcaggcttccggagggagccagcggccgaccccgcccgggagttgccggactcgcttctcagccccagcttctgcgggagggtactcggtgcagctggacgctctgcacttctcgactttcgaggacagcgccctgacagcgatgatggcgcaaaagaatttgtctccttctctccccgggagcatcttgcagccagtgcaggaggaaaacagcaaaacttcgcctcggtcccacagaagcgacggggaattttccccccattcccattacagtgactcggatgaggcaagt

SEQ ID NO: 10 - protein, 351 aa, Mus musculus
MSRLLHAEEWAEVKELGDHHRHPQPHHVPPLTPQPPATLQARDLPVYPAELSLLDSTDPRAWLTPTLQGLCTARAAQYLLHSPELGASEAAAPRDEADSQGELVRRSGCGGLSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPNVGEQPPPPTASCKNDHHHLRTASSYEGGAGASAVAGAQPAPGGGPRPTPPGPCRTRFSGPASSGGYSVQLDALHFPAFEDRALTAMMAQKDLSPSLPGGILQPVQEDNSKTSPRSHRSDGEFSPHSHYSDSDEAS

SEQ ID NO: 11 - DNA, 1053 bp, Mus musculus
  source 1..1053: mol_type = other DNA
atgtcccgcctgctgcatgcagaagagtgggctgaggtaaaagagttgggggaccaccatcgccatccccagccgcaccacgtcccgccgctgacgccacagccacctgctaccctgcaggcgagagaccttcccgtctacccggcagaactgtccctcctggatagcaccgacccacgcgcctggctgactcccactttgcagggcctctgcacggcacgcgccgcccagtatctgctgcattctcccgagctgggtgcctccgaggccgcggcgccccgggacgaggctgacagccagggtgagctggtaaggagaagcggctgtggcggcctcagcaagagccccgggcccgtcaaagtacgggaacagctgtgcaagctgaagggtggggttgtagtggacgagcttggctgcagccgccagcgagccccttccagcaaacaggtgaatggggtacagaagcaaaggaggctggcagcaaacgcaagggaacggcgcaggatgcacgggctgaaccacgccttcgaccagctgcgcaacgttatcccgtccttcaacaacgacaagaagctgtccaaatatgagaccctacagatggcccagatctacatcaacgctctgtcggagttgctgcagactcccaatgtcggagagcaaccgccgccgcccacagcttcctgcaaaaatgaccaccatcaccttcgcaccgcctcctcctatgaaggaggtgcgggcgcctctgcggtagctggggctcagccagccccgggagggggcccgagacctaccccgcccgggccttgccggactcgcttctcaggcccagcttcctctgggggttactcggtgcagctggacgctttgcacttcccagccttcgaggacagggccctaacagcgatgatggcacagaaggacctgtcgccttcgctgcccgggggcatcctgcagcctgtacaggaggacaacagcaaaacatctcccagatcccacagaagtgacggagagttttccccccactctcattacagtgactctgatgaggccagt

SEQ ID NO: 12 - DNA, 60 bp, Homo sapiens
  source 1..60: mol_type = genomic DNA
ctgccttctgagagcgctataaaggcagcggaagggtagtccgcggggcattccgggcgg

SEQ ID NO: 13 - DNA, 60 bp, Homo sapiens
  source 1..60: mol_type = genomic DNA
ctctaggcgggctctgctcttctttaaggagtcccacagggcctggcccgcccctgacct

SEQ ID NO: 14 - DNA, 2000 bp, Homo sapiens
  source 1..2000: mol_type = genomic DNA
taaagagttgtgagttgtgtaggtgagttgccatggagctacaaatatgagttgatattctgaaatcctagacagccatctccaaggttaagaaaaatccttatgcactcacttgcaaagatatccacagcatgctcttaatggagaaaaacaaagccttagatcaaatatgtaaagtaatttttagttttttgaaaaggtatgtttgggctatagataaatctgttcaaaaaacatgagagaagataataatggttgaaaggagacacagtgcttgccctcaagaagtttttgtctagtgagggagagagaacttgtatgtaaataaaattgtgttactaaggtagatagtgagaagtaacttaagagaggatcagataaggtattaagagaatacagaaaagggtctggattaattctgaacagcatcaaagaatgttcttgcaagagatagtgttttcaccagatcttgaaggtatggatgagggtatacagagtgagtatattcagattctactttaaaacaaatactttcctctgttgtagtggagttgagctatacatccaacaataatgaaaaaatacacgcatatatacatatatggagagagatacatattttagtacatgtagcaattgattaataaatgtacagtttaagtcgcatgcaaaaccttggagtgatagcaaacttcattgtaggatgtttagcagcatctctggtctctactcactagatcccaatagcatctccctaggtgtgacaaccaaaaatgtctccaggcattgacctctggaggcaaaaaaagccctttattaagaaccagtggtatacataagtaaaacatacacaagagattcctcccctcttctctgtatgtgaataaaaattgcaaagttcatgacctggattttccttttaggtttcttctttagtggttcttaacttcattgggtgaagtaagcctttgaagatctgttgaaagctgttgactcattcacttctcaggaaaacgcacatgctgactaccatttcagagaatttgcatcagggttctctggggaggagttctgagttctgtttccaggagctcgtagaattgtcatggtctgcatatgcaaggcaggtggattacggaaggttgatgtacagaggtctgtattttggagcctcttctgtatttacttcagaacactaacaatcaggcgagaatgttctggtttatcaaacccttccttctgcctttcatcttaaccatgcattagttttaacaaagttcatcccaacagaagacaaaacactgatgaggtaggatagctccagctcctcctccctctcttctagtcttgatttccatgtagtccagtttattccttccctgattgtccaggagaatgagaaaaagaaaaaacagagtctagtgggtaagaaagggccacctggacggcttgatttggattgtgaaataaaacacacacacatgcacacgtagaataagtggctaaaatctgagtaaatcgtgaactctctgtatcctccacccattgaatactcctaaaagactttctagaaattcaaggacttattaatatagaaacctggccattgttcctcttctcctccccatgtggtatgagagcacctgtggcaggctcccagagaccacggacctcttcctctaggcgggctctgctcttctttaaggagtcccacagggcctggcccgcccctgacctcgcaacccttgagattagtaacgggatgagtgaggatccgggtggcccctgcgtggcagccagtaagagtctcagccttcccggttcgggaaaggggaagaatgcaggaggggtaggatttctttcctgataggatcggttgggaaagaccgcagcctgtgtgtgtctttcccttcgaccaaggtgtctgttgctccgtaaataaaacgtcccactgccttctgagagcgctataaaggcagcggaagggtagtccgcggggc

SEQ ID NO: 15 - DNA, 1400 bp, Homo sapiens
  source 1..1400: mol_type = genomic DNA
ggctgctcggaaaacaggacgaggggagagacttgctcaataagctgaaagttctgcccccgagagggctgcgacagctgctggaatgtgcctgcagcgtccgcctcttggggacccgcggagcgcgccctgacggttccacgcctggcccgggggtctgcacctctcctccagtgcgcacctggagctgcgtcccgggtcaggtgcggggagggagggaatctcagtgtccccttccagccttgcaagcgcctttggcccctgccccagcccctcggtttgggggagatttcagaacgcggacagcgccctggctgcgggccataggggactgggtggaactcgggaagcccccagagcaggggcttactcgcttcaagtttggggaaccccgggcagcgggtgcaggccacgagacccgaaggttctcaggtgcccccctgcaggctggccgtgcgcgccgtggggcgcttgtcgcgagcgccgagggctgcaggacgcggaccagactcgcggtgcaggggggcctggctgcagctaacaggtgatcccgttctttctgttcctcgctcttcccctccgatcgtcctcgcttaccgcgtgtcctccctcctcgctgtcctctggctcgcaggtcatggcagcgccaggcggcaggtcggagccgccgcagctccccgagtacagctgcagctacatggtgtcgcggccggtctacagcgagctcgctttccagcaacagcacgagcggcgcctgcaggagcgcaagacgctgcgggagagcctggccaagtgctgcaggtagcggccgcgcgggcctgcgtagagagaagcggagcggggcgtccacgccttggggagggaagggcgtccccagcgggcgagagtggggtgcgggcggcggagcccctgggcgccagctgcttctcccagaggcccgactttcggtctccggtcctccacgccgcccttctggtgggagggtggctccatcagtctcgggcccgaaatgaacttacctgggaaactcgcctttggggagagtgggttctaggagccccgtctctctttttcctctctgaaggaaacttggagtgcctcttggggtacagtgggtccctgttgccttcttgggagcttgtttaaatgaaatgaatagggaaacccagctcttgaccaggaggagtccttgaaacactcaagctaagtaggcgggctaccattcagttagagaccaggatgcaagctagaacccaggggagcgcggggtgtgccaagtacttcatcagcaggctgtgggacccctggggaaagccaccctcagtctctaaacccaaacatgccgtaactagatgtcacaaacataaagaaattagagtttctaaaacctttcattatag

SEQ ID NO: 16 - DNA, 900 bp, Homo sapiens
  source 1..900: mol_type = genomic DNA
cggaaggttgatgtacagaggtctgtattttggagcctcttctgtatttacttcagaacactaacaatcaggcgagaatgttctggtttatcaaacccttccttctgcctttcatcttaaccatgcattagttttaacaaagttcatcccaacagaagacaaaacactgatgaggtaggatagctccagctcctcctccctctcttctagtcttgatttccatgtagtccagtttattccttccctgattgtccaggagaatgagaaaaagaaaaaacagagtctagtgggtaagaaagggccacctggacggcttgatttggattgtgaaataaaacacacacacatgcacacgtagaataagtggctaaaatctgagtaaatcgtgaactctctgtatcctccacccattgaatactcctaaaagactttctagaaattcaaggacttattaatatagaaacctggccattgttcctcttctcctccccatgtggtatgagagcacctgtggcaggctcccagagaccacggacctcttcctctaggcgggctctgctcttctttaaggagtcccacagggcctggcccgcccctgacctcgcaacccttgagattagtaacgggatgagtgaggatccgggtggcccctgcgtggcagccagtaagagtctcagccttcccggttcgggaaaggggaagaatgcaggaggggtaggatttctttcctgataggatcggttgggaaagaccgcagcctgtgtgtgtctttcccttcgaccaaggtgtctgttgctccgtaaataaaacgtcccactgccttctgagagcgctataaaggcagcggaagggtagtccgcggggcattccgggcggggcgcgagcagagacaggtgagtt

SEQ ID NO: 17 - DNA, 640 bp, Mus musculus
  source 1..640: mol_type = genomic DNA
gaaagaaaacaggagggtaagattcgtggggaaaatcctgtgtccttcccttccaccaaggtgtctctgttgctccataaataaaacgtcccaccgctttgagtgagagctataaaggcagcagaaaaactgtccccaggcattctggtcgggcagaactccggagcccaaacaggtgagtttatcacagaagagatgcagtcggctgcaccctgcccgctctgtcgtaactctctagggaaggtactggtcaggcgccgcgggcggggtgggcaggacagacaccagaaaaagcgagacaagaggaagctaagggtcagaccgggaaactcgcacctgtccttaaatgggactgccctgctgcagcccagagtgtgcgtggagctgtgtctccgatcgagtgaggggagtaccggaccctttccctgtcgcacaggccccagtccgattccggggagatttcaggacgcggacagcgccctgaattgcccagggaggccacccagcccaacacccaagccctcaaagtgaggctccctcccaggacagcggggattcccgggtatctggtacgggagactgcagtcctcgggtaccgtcctgcaggtggccggccgcagggacagtttgttgccagcgc

SEQ ID NO: 18 - DNA, 1347 bp, synthetic construct
  source 1..1347: mol_type = other DNA
taagtagaatacgtggttagaaaatccagcgctaagtagggtgacaagcaaggaactgttaaagccagtggagttgggttttttttttttttccaattttttcctcttcttttatttcagacaataattgctactgccatttcctatggggccaacttggaaaagaactacaatgctggcattgttaagtccatcccaagtgggtgagtgtggccttccttccagatggcactaataaactagggcagcactttgtttaaatggagctttttattactggttttgctttactatttattgatagtcttacttgcatgtgattttggggaaatgtcatgtgcactacacatcagacaaacagcgcatgtggggaccaagttcaggccatttcagtcaggacttctgtgagtccacggcctttgtctgtaagctctccgctgactcagggaaggaagtggtgagaaaggctggaaggtggacaggagccagataatgaagggcctcacacactgcattaaggagcttggattttaatctcatctggtaagagataaaggcaaagtggtggcttacattacctggctaatccaatcagctgacctaggtacctagaaatagattcagaggaaattaaagtagagatgaatgaagcttttgtttttgtttcttttttttttctcccaaggagagagtaagagatgaaagaagaaagaaaacaggagggtaagattcgtggggaaaatcctgtgtccttcccttccaccaaggtgtctctgttgctccataaataaaacgtcccaccgctttgagtgagagctataaaggcagcagaaaaactgtccccaggcattctggtcgggcagaactccggagcccaaacaggtgagtttatcacagaagagatgcagtcggctgcaccctgcccgctctgtcgtaactctctagggaaggtactggtcaggcgccgcgggcggggtgggcaggacagacaccagaaaaagcgagacaagaggaagctaagggtcagaccgggaaactcgcacctgtccttaaatgggactgccctgctgcagcccagagtgtgcgtggagctgtgtctccgatcgagtgaggggagtaccggaccctttccctgtcgcacaggccccagtccgattccggggagatttcaggacgcggacagcgccctgaattgcccagggaggccacccagcccaacacccaagccctcaaagtgaggctccctcccaggacagcggggattcccgggtatctggtacgggagactgcagtcctcgggtaccgtcctgcaggtggccggccgcagggacagtttgttgccagcgc

(19) United States
(12) Patent Application Publication
(10) Pub. No.: US 2026/0034248 A1
(43) Pub. Date: Feb. 5, 2026

(54) SLC26A4 REGULATORY ELEMENTS AND USES THEREOF

(71) Applicant: Decibel Therapeutics, Inc., Boston, MA (US)

(72) Inventors: Tyler GIBSON, Boston, MA (US); Monika KOWALCZYK, Somerville, MA (US); Gabriela PREGERNIG, Cambridge, MA (US); Jonathan B. SELLON, Newton, MA (US); Kathy SO, Boston, MA (US)

(21) Appl. No.: 19/099,853

(22) PCT Filed: Aug. 4, 2023
(86) PCT No.: PCT/US2023/071651

§ 371 Date: Jan. 30, 2025

Related U.S. Application Data
(60) Provisional application No. 63/395,565, filed on Aug. 5, 2022.
(60) Provisional application No. 63/453,856, filed on Mar. 22, 2023.

(51) Int. Cl. A61K 48/00; A61K 9/00; A61K 38/17; A61P 27/16; C12N 15/86

(52) U.S. Cl. CPC A61K 48/0058; A61K 9/0046; A61K 38/1709; A61K 48/0075; A61P 27/16; C12N 15/86; C12N 2750/14143

(57) ABSTRACT

The disclosure provides SL26A4 enhancers and SLC26A4 promoters, as well as vectors containing the same, that can increase gene expression in SLC26A4-expressing cells, such as interdental cells, root cells, spiral prominence cells, and vestibular supporting cells. The SLC26A4 enhancers and SLC26A4 promoters described herein may be operably linked to a polynucleotide, such as a transgene, encoding an expression product and used for the treatment of subjects having or at risk of developing hearing loss or vestibular dysfunction.

### SEQUENCE LISTING

The instant application contains a Sequence Listing which has been submitted electronically in XML file format and is hereby incorporated by reference in its entirety. Said XML copy, created on Jul. 31, 2023, is named 51124-098WO3_Sequence_Listing_7_31_23 and is 33,576 bytes in size.

### BACKGROUND

Hearing loss is the most common human sensory deficit, affecting nearly 15% of school-age children and one out of three people by age sixty-five. Congenital hearing loss occurs in about one out of every 500 newborn births in the United States every year. About 80% of congenital cases are due to mutations found in genes essential for hearing. One of these essential hearing genes is solute carrier family 26, member 4 (SLC26A4), which encodes Pendrin, a 780-amino acid member of solute carrier (SLC) family 26. Pendrin function may be lost or disrupted in patients with SLC26A4 mutations, and these patients may develop prelingual or postlingual nonsyndromic hearing loss or may be born with impaired hearing that progresses to profound deafness over time. Pendrin is expressed in the apical membranes of specialized epithelial cells of the inner ear (non-sensory epithelial cells of the cochlea, vestibular labyrinth, and endolymphatic sac as well as the saccule, utricle, and ampulla), thyroid (thyrocyte), kidney (renal collecting type B intercalated cell), airways, mammary gland, salivary duct, and liver. Within the inner ear, Pendrin regulates pH and fluid absorption by exchanging chloride and bicarbonate anions between the epithelium and the endolymphatic fluid compartment. There is currently no curative therapy for this population. Therefore, there is a need for a therapeutic to restore and/or rescue hearing loss progression in patients with these mutations.

### SUMMARY OF THE INVENTION

The invention provides compositions and methods for promoting the expression of a gene of interest, such as a gene that is endogenously expressed in SLC26A4-expressing cells, in specific cell types. The present invention features SLC26A4 promoters and enhancers that can be operably linked to a polynucleotide that can be transcribed to produce an expression product (e.g., a protein or an RNA molecule, such as an inhibitory RNA molecule) to induce expression of the expression product in SLC26A4-expressing cells. The SLC26A4 promoters and enhancers can be incorporated into nucleic acid vectors and administered to a subject, such as a human subject, to treat or prevent hearing loss (e.g., sensorineural hearing loss, such as pendrin-related hearing loss), Meniere's disease (e.g., hearing loss, tinnitus, or vestibular dysfunction associated with Meniere's disease), and/or vestibular dysfunction (e.g., pendrin-related vestibular dysfunction or vestibular dysfunction associated with damage to or loss of vestibular hair cells).

In a first aspect, the invention provides a polynucleotide containing an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 3 operably linked to a promoter, wherein when the distance between the enhancer and the promoter in the polynucleotide is less than 3 kilobases (3 kb) (e.g., about 3.0 kb, 2.75 kb, 2.5 kb, 2.25 kb, 2.0 kb, 1.75 kb, 1.5 kb, 1.25 kb, 1.0 kb, 900 bases, 800 bases, 700 bases, 600 bases, 500 bases, 400 bases, 300 bases, 200 bases, 100 bases, 50 bases, or less). In some embodiments, the distance between the enhancer and the promoter in the polynucleotide is less than 2 kb. In some embodiments, the distance between the enhancer and the promoter in the polynucleotide is less than 1 kb. In some embodiments, the distance between the enhancer and the promoter in the polynucleotide is less than 0.5 kb. In some embodiments, the distance between the enhancer and the promoter in the polynucleotide is less than 100 bases.

In another aspect, the invention provides a nucleic acid vector comprising the polynucleotide of the first aspect.

In another aspect, the invention provides a nucleic acid vector comprising a polynucleotide comprising an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 3.

In some embodiments of any of the foregoing aspects, the enhancer is operably linked to a promoter. In some embodiments, the promoter is a minimal promoter, a core promoter, or a constitutive promoter. In some embodiments, the promoter is a CAG promoter, a CBA promoter, an smCBA promoter, a CASI promoter, a dihydrofolate reductase (DHFR) promoter, a β-actin promoter, a phosphoglycerol kinase (PGK) promoter, an EF1α promoter, a β-globin promoter, a CMV promoter, an HSV promoter, or an SV40 promoter. In some embodiments, the promoter is a minimal β-globin promoter, a CMVmini promoter, a minCMV promoter, a CMV-TATA+INR promoter, a min CMV-T6 promoter, a minimal HSV ICP0 promoter, a truncated HSV ICP0 promoter, or an SV40 minimal promoter. In some embodiments, the promoter is a minimal promoter. In some embodiments, the promoter is a mammalian SLC26A4 promoter. In some embodiments, the SLC26A4 promoter is a human or murine SLC26A4 promoter. In some embodiments, the SLC26A4 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1. In some embodiments, the SLC26A4 promoter has the sequence of SEQ ID NO: 1. In some embodiments, the SLC26A4 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 12-16. In some embodiments, the SLC26A4 promoter has the sequence of any one of SEQ ID NOs: 12-16. In some embodiments, the SLC26A4 promoter has the sequence of SEQ ID NO: 12. In some embodiments, the SLC26A4 promoter has the sequence of SEQ ID NO: 13. In some embodiments, the SLC26A4 promoter has the sequence of SEQ ID NO: 14. In some embodiments, the SLC26A4 promoter has the sequence of SEQ ID NO: 15. In some embodiments, the SLC26A4 promoter has the sequence of SEQ ID NO: 16. In some embodiments, the SLC26A4 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17. In some embodiments, the SLC26A4 promoter has the sequence of SEQ ID NO: 17.

In another aspect, the invention provides a nucleic acid vector containing a polynucleotide including a SLC26A4 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1. In some embodiments, the SLC26A4 promoter has the sequence of SEQ ID NO: 1.

In another aspect, the invention provides a polynucleotide including a SLC26A4 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 operably linked to a polynucleotide that can be transcribed to produce an expression product. In some embodiments, the SLC26A4 promoter has the sequence of SEQ ID NO: 1.

In another aspect, the invention provides a nucleic acid vector containing a polynucleotide including a SLC26A4 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17. In some embodiments, the SLC26A4 promoter has the sequence of SEQ ID NO: 17.

In another aspect, the invention provides a polynucleotide including a SLC26A4 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17 operably linked to a polynucleotide that can be transcribed to produce an expression product. In some embodiments, the SLC26A4 promoter has the sequence of SEQ ID NO: 17.

In some embodiments of any of the foregoing aspects, the SLC26A4 promoter is operably linked to an enhancer. In some embodiments, the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or at least 85% sequence identity (e.g., 85%, 86%, 87%, 88%, 89%, 90%, 91%, 92%, 93%, 94%, 95%, 96%, 97%, 98%, 99%, or more, sequence identity) SEQ ID NO: 3.

In some embodiments of any of the foregoing aspects, the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2. In some embodiments of any of the foregoing aspects, the enhancer has the sequence of SEQ ID NO: 2.

In some embodiments of any of the foregoing aspects, the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 3. In some embodiments of any of the foregoing aspects, the enhancer has the sequence of SEQ ID NO: 3.

In some embodiments of any of the foregoing aspects, the enhancer is positioned 5′ of the promoter in the polynucleotide. In some embodiments of any of the foregoing aspects, the enhancer is positioned 3′ of the promoter in the polynucleotide. In some embodiments of any of the foregoing aspects, the enhancer is directly fused to the promoter. In some embodiments of any of the foregoing aspects, the enhancer is joined to the promoter by a nucleic acid linker of one to 100 nucleic acids (e.g., about 1, 5, 10, 15, 20, 30, 40, 50, 60, 70, 80, 90, or 100 nucleic acids).

In some embodiments of any of the foregoing aspects, the promoter is operably linked to a polynucleotide that can be transcribed to produce an expression product. In some embodiments of any of the foregoing aspects, the expression product is a heterologous expression product. In some embodiments of any of the foregoing aspects, the expression product is an expression product that is endogenously expressed in a SLC26A4-expressing cell. In some embodiments, the expression product is an expression product that is endogenously expressed in a SLC26A4-expressing inner ear cell. In some embodiments, the expression product is an expression product that is endogenously expressed in an interdental cell, a spiral prominence cell, a cochlear root cell, and/or a vestibular supporting cell (e.g., expressed in at least one of these cell types). In some embodiments of any of the foregoing aspects, the expression product is pendrin (e.g., mammalian pendrin). In some embodiments, the pendrin (e.g., the mammalian pendrin) is a wild-type isoform endogenously expressed in an inner ear of a mammal. In some embodiments, the pendrin has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 4 or SEQ ID NO: 5. In some embodiments, the pendrin has the sequence of SEQ ID NO: 4 or SEQ ID NO: 5. In some embodiments of any of the foregoing aspects, the expression product is Atoh1 (e.g., mammalian Atoh1). In some embodiments, the Atoh1 (e.g., the mammalian Atoh1) is a wild-type isoform endogenously expressed in an inner ear of a mammal. In some embodiments, the Atoh1 has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 8 or SEQ ID NO: 10. In some embodiments, the Atoh1 has the sequence of SEQ ID NO: 8 or SEQ ID NO: 10. In some embodiments of any of the foregoing aspects, the expression product is a protein, a short hairpin RNA (shRNA), an antisense oligonucleotide (ASO), a component of a gene editing system (e.g., a nuclease, such as a CRISPR Associated Protein 9 (Cas9), Transcription Activator-Like Effector Nuclease (TALEN), or Zinc Finger Nuclease (ZFN), or a guide RNA (gRNA)), or a microRNA.

In some embodiments of any of the foregoing aspects, the polynucleotide contains an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 and an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 3. In some embodiments, the polynucleotide contains an enhancer that has the sequence of SEQ ID NO: 2 and an enhancer that has the sequence of SEQ ID NO: 3. In some embodiments of any of the foregoing aspects, the polynucleotide contains, in 5′ to 3′ order, an enhancer that has the sequence of SEQ ID NO: 2, an enhancer that has the sequence of SEQ ID NO: 3, and a promoter that has the sequence of SEQ ID NO: 17. In some more specific embodiments of any of the foregoing aspects, the polynucleotide contains the sequence of SEQ ID NO: 18.

In some embodiments of any of the foregoing aspects, the polynucleotide contains two or more copies of an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 and/or two or more copies of an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) SEQ ID NO: 3 (e.g., two or more copies of one or both enhancers). In some embodiments, the polynucleotide contains two or more copies of an enhancer that has the sequence of SEQ ID NO: 2 and/or SEQ ID NO: 3 (e.g., two or more copies of one or both enhancers).

In some embodiments of any of the foregoing aspects, the nucleic acid vector is a viral vector, plasmid, cosmid, or artificial chromosome. In some embodiments, the nucleic acid vector is a viral vector. In some embodiments, the viral vector is an adeno-associated virus (AAV), an adenovirus, or a lentivirus. In some embodiments, the viral vector is an AAV vector. In some embodiments, the AAV vector has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ, DJ/8, DJ/9, 7m8, PHP.B, PHP.eB, or PHP.S capsid. In some embodiments, the AAV vector has an AAV1 capsid. In some embodiments, the AAV vector has an AAV2 capsid. In some embodiments, the AAV vector has an AAV2quad(Y-F) capsid. In some embodiments, the AAV vector has an AAV6 capsid. In some embodiments, the AAV vector has an AAV8 capsid. In some embodiments, the AAV vector has an AAV9 capsid. In some embodiments, the AAV vector has an Anc80 capsid. In some embodiments, the AAV vector has Anc80L65 capsid. In some embodiments, the AAV vector has a DJ capsid. In some embodiments, the AAV vector has a DJ/9 capsid. In some embodiments, the AAV vector has a 7m8 capsid. In some embodiments, the AAV vector has a PHP.B capsid. In some embodiments, the AAV vector has a PHP.S capsid. In some embodiments, the AAV vector has a PHP.eB capsid. In some embodiments, the AAV vector has an AAV3 capsid. In some embodiments, the AAV vector has an AAV4 capsid. In some embodiments, the AAV vector has an AAV5 capsid. In some embodiments, the AAV vector has an AAV7 capsid.

In another aspect, the invention provides a composition containing the nucleic acid vector of any of the foregoing aspects and embodiments. In some embodiments, the composition further includes a pharmaceutically acceptable carrier, diluent, or excipient.

In another aspect, the invention provides a cell containing the polynucleotide or the nucleic acid vector of any of the foregoing aspects and embodiments. In some embodiments, the cell is a SLC26A4-expressing cell. In some embodiments, the cell is a SLC26A4-expressing inner ear cell. In some embodiments, the cell is a mammalian cell. In some embodiments, the mammalian cell is a human cell. In some embodiments, the cell is an interdental cell, spiral prominence cell, cochlear root cell, or vestibular supporting cell.

In another aspect, the invention provides a method of expressing an expression product in a cell, the method including the step of contacting the cell with the nucleic acid vector or composition of any of the foregoing aspects and embodiments. In some embodiments, the cell is an inner ear cell. In some embodiments, the cell is an SLC26A4-expressing cell. In some embodiments, the cell is an SLC26A4-expressing inner ear cell. In some embodiments, the SLC26A4-expressing inner ear cell is an interdental cell, spiral prominence cell, cochlear root cell, or vestibular supporting cell. In some embodiments, the cell is a mammalian cell. In some embodiments, the mammalian cell is a human cell. In some embodiments, the contacting is in a subject (e.g., in vivo).

In another aspect, the invention provides a method of treating a subject having or at risk of developing hearing loss (e.g., sensorineural hearing loss), the method including the step of administering to an inner ear of the subject a therapeutically effective amount of the nucleic acid vector or the composition of any of the foregoing aspects and embodiments. In some embodiments, the hearing loss is pendrin-related hearing loss. In some embodiments, the expression product is pendrin. In some embodiments, the pendrin-related hearing loss is hearing loss associated with Pendred syndrome or DFNB4.

In another aspect, the invention provides a method of treating hearing loss associated with Meniere's disease in a subject in need thereof, the method including the step of administering to an inner ear of the subject an effective amount of the nucleic acid vector or the composition of any of the foregoing aspects and embodiments. In some embodiments, the expression product is pendrin.

In another aspect, the invention provides a method of treating tinnitus associated with Meniere's disease in a subject in need thereof, the method including the step of administering to an inner ear of the subject an effective amount of the nucleic acid vector or the composition of any of the foregoing aspects and embodiments. In some embodiments, the expression product is pendrin.

In another aspect, the invention provides a method of treating vestibular dysfunction associated with Meniere's disease in a subject in need thereof, the method including the step of administering to an inner ear of the subject an effective amount of the nucleic acid vector or the composition of any of the foregoing aspects and embodiments. In some embodiments, the expression product is pendrin or Atoh1. In some embodiments, the vestibular dysfunction is vertigo.

In another aspect, the invention provides a method of treating a subject having or at risk of developing vestibular dysfunction, the method including the step of administering to an inner ear of the subject a therapeutically effective amount of the nucleic acid vector or composition of any of the foregoing aspects and embodiments. In some embodiments, the vestibular dysfunction is pendrin-related vestibular dysfunction. In some embodiments, the expression product is pendrin. In some embodiments, the pendrin-related vestibular dysfunction is vestibular dysfunction associated with Pendred syndrome or DFNB4. In some embodiments, the expression product is pendrin or Atoh1. In some embodiments, the vestibular dysfunction is vertigo, dizziness, imbalance (e.g., loss of balance or a balance disorder), oscillopsia, or bilateral vestibulopathy. In some embodiments, the vestibular dysfunction is associated with damage to or loss of vestibular hair cells. In some embodiments, the damage to or loss of vestibular hair cells is associated with age (i.e., the vestibular dysfunction is age-related vestibular dysfunction), exposure to an ototoxic (e.g., vestibulotoxic) drug (i.e., the vestibular dysfunction is ototoxic drug-induced vestibular dysfunction), a disease or infection (i.e., the vestibular dysfunction is disease or infection-related vestibular dysfunction), or head trauma (i.e., the vestibular dysfunction is head trauma-related vestibular dysfunction). In some embodiments, the ototoxic drug is an aminoglycoside (an aminoglycoside antibiotic, e.g., gentamycin, neomycin, streptomycin, tobramycin, kanamycin, vancomycin, amikacin, dibekacin, or netilmicin), viomycin, an antineoplastic drug (e.g., a platinum-containing chemotherapeutic agent, such as cisplatin, carboplatin, or oxaliplatin, or another chemotherapeutic agent, such as a nitrogen mustard or vincristine), a loop diuretic (e.g., ethacrynic acid or furosemide), a salicylate, or quinine.

In another aspect, the invention provides a method of inducing or increasing vestibular hair cell regeneration (i.e., inducing or increasing differentiation of a vestibular supporting cell into a vestibular hair cell), the method including the step of contacting a vestibular supporting cell with the nucleic acid vector or the composition of any of the foregoing aspects and embodiments. In some embodiments, the expression product is Atoh1. In some embodiments, the contacting is in vivo (e.g., in a subject). In some embodiments, the subject has or is at risk of developing vestibular dysfunction.

In another aspect, the invention provides a method of inducing or increasing maturation of a vestibular hair cell (e.g., a regenerated vestibular hair cell), the method including the step of contacting a vestibular supporting cell with the nucleic acid vector or the composition of any of the foregoing aspects and embodiments. In some embodiments, the expression product is Atoh1. In some embodiments, the contacting is in vivo (e.g., in a subject). In some embodiments, the subject has or is at risk of developing vestibular dysfunction.

In another aspect, the invention provides a method of improving function of an SLC26A4-expressing cell, the method including the step of contacting the SLC26A4-expressing cell with the nucleic acid vector or the composition of any of the foregoing aspects and embodiments. In some embodiments, the contacting is in vivo (e.g., in a subject). In some embodiments, the subject has or is at risk of developing hearing loss (e.g., sensorineural hearing loss) or vestibular dysfunction.

In some embodiments of any of the foregoing aspects, the method further includes evaluating the hearing of the subject prior to administering the nucleic acid vector or composition.

In some embodiments of any of the foregoing aspects, the method further includes evaluating the hearing of the subject after administering the nucleic acid vector or composition.

In some embodiments of any of the foregoing aspects, the method further includes evaluating the vestibular function of the subject prior to administering the nucleic acid vector or composition.

In some embodiments of any of the foregoing aspects, the method further includes evaluating the vestibular function of the subject after administering the nucleic acid vector or composition.

In some embodiments of any of the foregoing aspects, the nucleic acid vector or composition is locally administered. In some embodiments, the nucleic acid vector or composition is administered to the inner ear. In some embodiments, the nucleic acid vector or composition is administered to the middle ear. In some embodiments, the nucleic acid vector or composition is administered transtympanically or intratympanically. In some embodiments, the nucleic acid vector or composition is administered into the perilymph. In some embodiments, the nucleic acid vector or composition is administered into the endolymph. In some embodiments, the nucleic acid vector or composition is administered to or through the oval window. In some embodiments, the nucleic acid vector or composition is administered to or through the round window. In some embodiments, the nucleic acid vector or composition is administered to a semicircular canal.

In some embodiments of any of the foregoing aspects, the nucleic acid vector or composition is administered in an amount sufficient to prevent or reduce hearing loss, delay the development of hearing loss, slow the progression of hearing loss, improve hearing, increase or induce expression of an expression product in an SLC26A4-expressing cell, reduce tinnitus, improve vestibular function, reduce vertigo, improve balance, increase vestibular hair cell numbers, inhibit or slow the progression of vestibular dysfunction, reduce the feeling of fullness in the ear, increase vestibular hair cell regeneration, induce or increase the differentiation of vestibular supporting cells into vestibular hair cells, increase or induce vestibular hair cell maturation (e.g., the maturation of regenerated vestibular hair cells), or improve the function of an SLC26A4-expressing cell (e.g., an SLC26A4-expressing inner ear cell).

In some embodiments of any of the foregoing aspects, the subject is a human subject.

In another aspect, the invention provides a kit including the polynucleotide, nucleic acid vector, or composition of any of the foregoing aspects and embodiments.

### Definitions

As used herein, the term “about” refers to a value that is within 10% above or below the value being described.

As used herein, “administration” refers to providing or giving a subject a therapeutic agent (e.g., a nucleic acid vector containing a SLC26A4 enhancer and/or a SLC26A4 promoter), by any effective route. Exemplary routes of administration are described herein below.

As used herein, the phrase “administering to the inner ear” refers to providing or giving a therapeutic agent described herein to a subject by any route that allows for transduction of inner ear cells. Exemplary routes of administration to the inner ear include administration into the perilymph or endolymph, such as to or through the oval window, round window, or semicircular canal (e.g., horizontal canal), or by transtympanic or intratympanic injection, e.g., administration to a SLC26A4-expressing inner ear cell.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally occurring amino acids in table 1, below.

TABLE 1
Representative physicochemical properties
of naturally occurring amino acids

| | | | | Electrostatic | |
|---|---|---|---|---|---|
| | 3 | 1 | Side- | character at | |
| | Letter | Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic | Glu | E | polar | anionic | intermediate |
| acid | | | | | |
| Glutamine | Gln | Q | polar | neutral | intermediate |
| Glycine | Gly | G | nonpolar | neutral | small |
| Histidine | His | H | polar | Both | large |
| | | | | neutral | |
| | | | | and | |
| | | | | cationic | |
| | | | | forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L, and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the terms “effective amount,” “therapeutically effective amount,” and a “sufficient amount” of a composition, vector construct, or viral vector described herein refer to a quantity sufficient to, when administered to the subject, including a mammal, for example a human, effect beneficial or desired results, including clinical results, and, as such, an “effective amount” or synonym thereto depends upon the context in which it is being applied. For example, in the context of treating hearing loss (e.g., hearing loss associated with DFNB4 or Pendred syndrome), it is an amount of the composition, vector construct, or viral vector sufficient to achieve a treatment response as compared to the response obtained without administration of the composition, vector construct, or viral vector. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition, vector construct, or viral vector of the present disclosure is an amount which results in a beneficial or desired result in a subject as compared to a control. As defined herein, a therapeutically effective amount of a composition, vector construct, or viral vector of the present disclosure may be readily determined by one of ordinary skill by routine methods known in the art. Dosage regimen may be adjusted to provide the optimum therapeutic response.

As used herein, the term “endogenous” refers to a molecule (e.g., a polypeptide, nucleic acid, or cofactor) that is found naturally in a particular organism (e.g., a human) or in a particular location within an organism (e.g., an organ, a tissue, or a cell, such as a human cell, e.g., a human cochlear supporting cell).

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. The term “expression product” refers to a protein or RNA molecule produced by any of these events.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell, e.g., a human cochlear supporting cell). Exogenous materials include those that are provided from an external source to an organism or to cultured matter extracted therefrom.

As used herein, the term “heterologous” refers to a combination of elements that is not naturally occurring. For example, a heterologous transgene refers to a transgene that is not naturally expressed by the promoter to which it is operably linked.

As used herein, the terms “increasing” and “decreasing” refer to modulating resulting in, respectively, greater or lesser amounts, of function, expression, or activity of a metric relative to a reference. For example, subsequent to administration of a composition in a method described herein, the amount of a marker of a metric (e.g., transgene expression level or auditory brainstem response) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration, administration directly to the inner or middle ear (e.g., injection through the oval window or round window membrane or transtympanic or intratympanic injection), and administration to a mucous membrane of the subject, wherein the administration is intended to have a local and not a systemic effect.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker polynucleotide (e.g., an intervening non-coding polynucleotide) or may be operably linked to one another with no intervening nucleotides present.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the term “polynucleotide” refers to a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. The term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated.

As used herein, the term “promoter” refers to a recognition site on DNA that is bound by an RNA polymerase. The polymerase drives transcription of the transgene.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows: 100 multiplied by (the fraction X/Y)

$$100 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the terms “Pendrin” and “SLC26A4” refer to a protein encoded by the SLC26A4 gene and to the gene encoding this protein, respectively. SLC26A4 is a member of solute carrier family 26. Mutations in SLC26A4 cause either syndromic or non-syndromic hearing loss. The terms “Pendrin” and “SLC26A4” also refer to variants of wild-type Pendrin and nucleic acids encoding the same, respectively, such as variant proteins having at least 85% sequence identity (e.g., 85%, 86%, 87%, 88%, 89%, 90%, 91%, 92%, 93%, 94%, 95%, 96%, 97%, 98%, 99%, or 99.9% identity, or more sequence identity) to the amino acid sequence of a wild-type Pendrin protein (e.g., SEQ ID NO: 4 or SEQ ID NO: 5) or polynucleotides having at least 85% sequence identity (e.g., 85%, 86%, 87%, 88%, 89%, 90%, 91%, 92%, 93%, 94%, 95%, 96%, 97%, 98%, 99%, or 99.9% identity, or more sequence identity) to the nucleic acid sequence of a wild-type SLC26A4 gene (e.g., SEQ ID NO: 6 or SEQ ID NO: 7) or a codon-optimized sequence thereof, provided that the Pendrin analog encoded retains the therapeutic function of wild-type (WT) Pendrin (e.g., the ability to transport negatively charged ions, such as chloride, iodide, and bicarbonate, across cell membranes).

As used herein, the term “SLC26A4 enhancer” refers to a polynucleotide that can be operably linked to a promoter (e.g., a SLC26A4 promoter, a minimal promoter, a core promoter, or a constitutive promoter) to regulate gene expression in SLC26A4-expressing cells. SLC26A4 enhancers for use in the compositions and methods described herein have at least 85% sequence identity to (e.g., 85%, 86%, 87%, 88%, 89%, 90%, 91%, 92%, 93%, 94%, 95%, 96%, 97%, 98%, 99%, or 99.9% identity, or more sequence identity) to SEQ ID NO: 2 or SEQ ID NO: 3. The SLC26A4 enhancers described herein can be operably linked to a promoter that is operably linked to a polynucleotide encoding an expression product to increase the expression level of the expression product in SLC26A4-expressing cells and/or increase the number of SLC26A4-expressing cells in which the expression product is expressed.

As used herein, the term “SLC26A4 promoter” refers to a polynucleotide that is capable of expressing a transgene specifically in SLC26A4-expressing cells, or a variant thereof, such as a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to a SLC26A4 promoter described herein (e.g., a SLC26A4 promoter or enhancer-promoter provided in Table 3). A SLC26A4 promoter for use in the compositions and methods described herein can have at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1, at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17, or at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 12-16.

As used herein, the term “SLC26A4-expressing cell” refers to a cell type in the body that is known to endogenously express SLC26A4. SLC26A4-expressing cells include cells of the breast (adipocytes, vascular cells, luminal epithelial cells, fibroblasts, dendritic cells, macrophages, basal myoepithelial cells, pericytes, and smooth muscle cells); esophagus (adipocytes, lymphatic cells, vascular cells, epithelial cells of the esophageal mucosa (basal, suprabasal, squamous), fibroblasts (mucosal and muscle), mucous cells, myofibroblasts of the esophageal mucosa, neuronal cells of the esophageal muscle layer, Schwann cells, myocytes (smooth muscle), immune cells (dendritic cells, macrophages, T cells, mast cells), pericytes, and smooth muscle cells); heart (adipocytes, lymphatic cells, vascular cells, fibroblasts, Schwann cells, myocytes (cardiac, cytoplasmic), immune cells (dendritic cells, macrophages, T cells, mast cells), pericytes, and smooth muscle cells); lung (lymphatic cells, vascular cells, epithelial cells (Alveolar Type I, Type II, Basal, Ciliated, and Club), fibroblasts, immune cells (dendritic cells, macrophages, T cells, alveolar macrophages), pericytes, and smooth muscle cells); skeletal muscle (lymphatic cells, vascular cells, fibroblasts, dendritic cells, macrophages, satellite cells, myocytes (skeletal muscle, cytoplasmic), pericytes, and smooth muscle cells); prostate (lymphatic cells, vascular cells, epithelial cells (Hillock, luminal), fibroblasts, dendritic cells, macrophages, myocytes (smooth muscle)); skin (vascular cells, epithelial cells (basal keratinocyte, mature keratinocyte, suprabasal keratinocyte), fibroblasts, sebaceous gland cells, and sweat gland cells); kidney (cortical and medullar cells (intercalated cells)); and thyroid (follicular and parafollicular cells) and SLC26A4-expressing inner ear cells.

As used herein, the term “SLC26A4-expressing inner ear cell” refers to a cell within the inner ear that endogenously expresses SLC26A4. SLC26A4-expressing cells within the ear are found in both the cochlea and the vestibule. Cochlear SLC26A4-expressing cells include root cells, spindle cells, inner sulcus cells, outer sulcus cells, spiral prominence cells, interdental cells, macrophages, Reissner's membrane, Deiters' cells, vascular cells, marginal cells, and intermediate cells. Vestibular SLC26A4-expressing cells include non-sensory epithelial cells. Additional SLC26A4-expressing inner ear cells include endolymphatic sac mitochondria-rich cells of the endolymphatic sac and endolymphatic duct.

As used herein, the term “pendrin-related hearing loss” refers to a disease or condition that features hearing loss associated with a mutation in SLC26A4, such as DFNB4, which is characterized by prelingual or postlingual hearing loss that may be accompanied by an enlarged vestibular aqueduct, and Pendred syndrome, which is characterized by enlargement of the thyroid gland (called a goiter), severe to profound hearing loss (often from birth), and other abnormalities of the inner ear, including an enlarged vestibular aqueduct.

As used herein, the term “pendrin-related vestibular dysfunction” refers to a disease or condition that features vestibular dysfunction (e.g., vertigo, dizziness, or imbalance or balance loss) associated with a mutation in SLC26A4, such as DFNB4 and Pendred syndrome.

As used herein, the term “pharmaceutical composition” refers to a mixture containing a therapeutic agent, optionally in combination with one or more pharmaceutically acceptable excipients, diluents, and/or carriers, to be administered to a subject, such as a mammal, e.g., a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response, and other problem complications commensurate with a reasonable benefit/risk ratio.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject.

As used herein, the terms “subject” and “patient” refer to an animal (e.g., a mammal, such as a human). A subject to be treated according to the methods described herein may be one who has been diagnosed with hearing loss (e.g., pendrin-related hearing loss), vestibular dysfunction (e.g., pendrin-related vestibular dysfunction or vestibular dysfunction associated with loss of vestibular hair cells),or Meniere's disease or one at risk of developing these conditions (e.g., due to a mutation in SLC26A4 or exposure to an insult that can cause vestibular hair cell damage or death, such as exposure to an ototoxic drug, head trauma, or aging). Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the terms “transcription regulatory element” and “regulatory sequence” refer to a polynucleotide that controls, at least in part, the transcription of a gene of interest. Transcription regulatory elements may include promoters, enhancers, and other polynucleotides (e.g., polyadenylation signals) that control or help to control gene transcription. Examples of transcription regulatory elements are described, for example, in Lorence, Recombinant Gene Expression: Reviews and Protocols (Humana Press, New York, NY, 2012).

As used herein, the term “transfection” refers to any of a wide variety of techniques commonly used for the introduction of exogenous DNA into a prokaryotic or eukaryotic host cell, e.g., electroporation, lipofection, calcium phosphate precipitation, DEAE-dextran transfection, Nucleofection, squeeze-poration, sonoporation, optical transfection, magnetofection, impalefection and the like.

As used herein, the terms “transduction” and “transduce” refer to a method of introducing a vector construct or a part thereof into a cell. Wherein the vector construct is contained in a viral vector such as for example an AAV vector, transduction refers to viral infection of the cell and subsequent transfer and integration of the vector construct or part thereof into the cell genome.

As used herein, “treatment” and “treating” in reference to a disease or condition, refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “vector” refers to a nucleic acid vector, e.g., a DNA vector, such as a plasmid, cosmid, or artificial chromosome, an RNA vector, a virus, or any other suitable replicon (e.g., viral vector). A variety of vectors have been developed for the delivery of polynucleotides encoding exogenous proteins into a prokaryotic or eukaryotic cell. Examples of such expression vectors are described in, e.g., Gellissen, Production of Recombinant Proteins: Novel Microbial and Eukaryotic Expression Systems (John Wiley & Sons, Marblehead, MA, 2006). Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of transgene as described herein include vectors that contain regulatory sequences, such as promoter and enhancer regions, which direct gene transcription. Other useful vectors for expression of a transgene contain polynucleotide sequences that enhance the rate of translation of the transgene or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “wild-type” refers to a genotype with the highest frequency for a particular gene in a given organism.

### BRIEF DESCRIPTION OF THE DRAWINGS

FIG. 1 is a plasmid map of plasmid P1236.

FIG. 2 is a plasmid map of plasmid P1240.

FIGS. 3A-3C are a series of images showing inner ear tropism of AAV1 containing a green fluorescent protein (GFP) transgene driven by a ubiquitous promoter (CMV). Section of mouse inner ear cochlea and vestibular organs demonstrating expression of the enhanced green fluorescent protein (EGFP) transgene (FIG. 3A). AAV1 tropism, as determined by EGFP expression, in the cochlea includes but is not limited to the spiral ganglion neurons (SGN), Reissner's membrane (RM), fibrocytes (F), spiral prominence cells (SP), and root cells (RC) (FIG. 3B). AAV1 tropism in the vestibular system includes but is not limited to hair cells (HC), supporting cells (SC), mesenchymal cells (MC), and cells comprising the roof of the otolith organs (FIG. 3C).

FIGS. 4A-4D are a series of images demonstrating a lack of EGFP transgene expression in the inner ear organs when driven by the murine SLC26A4 promoter of SEQ ID NO: 1 without any enhancer sequences. Sections of mouse inner ear cochlea and vestibular organs demonstrating no EGFP expression (FIGS. 4A and 4C, respectively). No EGFP expression was found in sections of mouse inner ear cochlea (square in FIG. 4A; higher resolution in FIG. 4B), or vestibular organs (rectangle in FIG. 4C; higher resolution in FIG. 4D), respectively. Scale bars, 100 μm.

FIGS. 5A-5H are a series of images demonstrating restriction of EGFP transgene expression to specific cell types of the inner ear when driven by the murine E2 enhancer element of SEQ ID NO: 2 fused directly to the murineSLC26A4 promoter of SEQ ID NO: 1. Sections of the mouse inner ear cochlea and vestibular organs across two different animals (top and bottom rows) demonstrating EGFP expression. A lower magnification view of the section from animal #1 is shown if FIG. 5A. Higher magnification view of: the leftmost rectangle is shown in FIG. 5B; the uppermost rectangle is shown in FIG. 5C; and the rightmost rectangle is shown in FIG. 5D. A lower magnification view of the section from animal #2 is shown in FIG. 5E. Higher magnification view of: the upper left rectangle is shown in FIG. 5F; the upper right rectangle is shown in FIG. 5G; and the bottommost rectangle is shown in FIG. 5H. Expression of EGFP was restricted to interdental cells (ID) (FIG. 5B), spiral prominence cells (SP) (FIGS. 5D, 5F and 5G), and root cells (RC) (FIG. 5G) of the cochlea in both animals. Expression of EGFP was restricted to supporting cells (SC) in the otolith organs of the vestibule (FIG. 5H). Scale bars, 100 μm.

FIGS. 6A-6C are a series of images demonstrating restriction of EGFP transgene expression to specific cell types of the inner ear when driven by the murine E6 enhancer element of SEQ ID NO: 3 fused directly to the murine SLC26A4 promoter of SEQ ID NO: 1. Section of the mouse inner ear and vestibular organs demonstrating EGFP expression (FIG. 6A) and higher magnifications thereof (upper rectangle, FIG. 6B; lower rectangle, FIG. 6C). Expression of EGFP is shown in spiral prominence (SP) and root cells (RC) of the cochlea (FIG. 6B). Weak expression of EGFP was observed in the supporting cells (SC) of the vestibular organs (FIG. 6C). Scale bars, 100 μm.

FIG. 7 is a plasmid map of plasmid P1669.

FIG. 8 is a plasmid map of plasmid P1670.

FIGS. 9A-9B are a series of images evaluating pendrin and EGFP expression. FIG. 9A is an image of a whole mount view of a mouse lateral wall explant stained with an antibody specific for pendrin. The different layers of the lateral wall are indicated in the image. FIG. 9B is a series of images of a whole mount view of a mouse lateral wall transduced with an AAV vector expressing EGFP under control of the ubiquitous CMV promoter and stained with antibodies specific for pendrin. Images showing pendrin staining alone, GFP alone, and a combination of pendrin and GFP are shown as indicated.

FIGS. 10A-10C are a series of images depicting a whole mount view of mouse lateral wall explants transduced with an AAV1 viral vector expressing EGFP under the control of: the murine minimal SLC26A4 promoter and both the murine E2 and E6 enhancers (FIG. 10A), the murine core SLC26A4 promoter and both the murine E2 and E6 enhancers (FIG. 10B), or the murine core SLC26A4 promoter and the only the murine E2 enhancer (FIG. 10C) and counter-stained with an antibody specific for pendrin. The top row of each figure depicts both pendrin and GFP. The second row depicts only the pendrin staining in that micrograph. The third row depicts only GFP. The fourth row depicts only the GFP with an adjusted gain to better show GFP in FIG. 10C.

FIG. 11 is a series of different magnification images of the same field depicting EGFP expression in the cochlea following in vivo administration to a pendrin knockout mouse of an AAV1 vector containing a EGFP expression cassette encoding the E2 enhancer (SEQ ID NO: 2) fused directly to the 5′ end of the E6 enhancer (SEQ ID NO: 3) fused directly to the 5′ end of the murine SLC26A4 minimal promoter (SEQ ID NO: 17) (see plasmid P1669; FIG. 7). Panel A is a micrograph at lowest magnification. The area indicated with a box labeled “B” in panel A is shown in higher magnification in panel B. The boxes labeled “C” and “D” in panel B are shown in higher magnification in panels C and D, respectively.

FIG. 12 is a micrograph depicting EGFP expression in the cochlea following in vivo administration of an AAV1 vector containing an EGFP expression cassette encoding the E2 enhancer (SEQ ID NO: 2) fused directly to the 5′ end of the E6 enhancer (SEQ ID NO: 3) fused directly to the 5′ end of the murine SLC26A4 minimal promoter (SEQ ID NO: 17) (plasmid P1669; FIG. 7) to a wild-type non-human primate. Arrows point to cells expressing nuclear EGFP.

### DETAILED DESCRIPTION

Described herein are compositions and methods for inducing gene expression specifically in SLC26A4-expressing cells or a subpopulation thereof (e.g., SLC26A4-expressing inner ear cells, such as interdental cells, spiral prominence cells, root cells, and vestibular supporting cells). The invention features SLC26A4 enhancers that can be operably linked to a promoter to induce transgene expression in SLC26A4-expressing cells (e.g., SLC26A4-expressing inner ear cells). The SLC26A4 enhancers can also increase gene expression level and the number of SLC26A4-expressing cells in which the gene expression can be detected. In some embodiments, the SLC26A4 enhancers may reduce or minimize off-target expression in non-SLC26A4-expressing cells (e.g., when operably linked to a constitutive promoter). Accordingly, the SLC26A4 enhancers can be operably linked to a promoter that is, in turn, operably linked to a polynucleotide encoding an expression product (e.g., a polynucleotide encoding a protein or a polynucleotide that can be transcribed to produce an RNA molecule, such as an inhibitory RNA molecule) to induce expression of the expression product in SLC26A4-expressing cells with minimal off target expression in cells that do not endogenously express SLC26A4 (e.g., cochlear hair cells). The invention also features an SLC26A4 promoter that can be used to induce expression of an expression product to which it is operably linked specifically in SLC26A4-expressing cells. One or more of the SLC26A4 enhancers described herein can be operably linked to the SLC26A4 promoter described herein. The invention also features nucleic acid vectors containing one or more SLC26A4 enhancers operably linked to a promoter (e.g., a SLC26A4 promoter, such as an SLC26A4 promoter or enhancer-promoter provided in Table 3, a minimal promoter, a core promoter, or a constitutive promoter) that is operably linked to a polynucleotide encoding an expression product, and nucleic acid vectors containing a SLC26A4 promoter (e.g., a SLC26A4 promoter having at least 85% sequence identity to SEQ ID NO: 1) operably linked to a polynucleotide encoding an expression product, and methods of using these vectors to treat hearing loss (e.g., pendrin-related hearing loss), vestibular dysfunction (e.g., imbalance or loss of balance associated with pendrin-related vestibular dysfunction or loss of vestibular hair cells), or Meniere's disease.

### Pendrin

Pendrin is an anion exchange protein encoded by the SLC26A4 gene and is a member of solute carrier family 26. Mutations in SLC26A4 have been associated with both nonsyndromic and syndromic forms of hearing loss. Dozens of SLC26A4 mutations have been identified in subjects with nonsyndromic hearing loss (loss of hearing that is not associated with signs and symptoms affecting other parts of the body) called DFNB4. This form of hearing loss can be prelingual or postlingual, and subjects with DFNB4 often have an enlarged vestibular aqueduct. Over 150 mutations in SLC26A4 are associated with Pendred syndrome, which is characterized by enlargement of the thyroid gland (a goiter), hearing loss, and other abnormalities of the inner ear, including an enlarged vestibular aqueduct. Pendred syndrome is the most common form of syndromic deafness, and subjects with Pendred syndrome often begin to lose their hearing at birth or by the age of three and exhibit hearing loss that worsens over time, progressing to profound deafness in some subjects. There are no curative treatments for pendrin-related hearing loss, and supportive therapies are typically aimed at improving hearing, for example, by using hearing aids.

Gene therapy has recently emerged as an attractive therapeutic approach for treating hearing loss, particularly hearing loss caused by a mutation in a gene expressed in the inner ear, since delivering a wild-type version of the mutated gene could potentially improve or restore hearing. However, there are many genes associated with hearing loss and they are expressed in a variety of different cell types. To avoid off-target effects, it is best to induce gene expression only in those cells that endogenously express the gene. This can prove challenging for a gene such as SLC26A4, which is expressed in an assortment of different cell types in the cochlea and vestibule.

The present invention is based, in part, on the discovery of SLC26A4 enhancers and promoters that can be used to induce gene expression in SLC26A4-expressing cells while minimizing off-target expression in non-SLC26A4-expressing cells. The SLC26A4 enhancers can be operably linked to a promoter, such as an SLC26A4 promoter, which can, in turn, be operably linked to a polynucleotide encoding an expression product (e.g., a polynucleotide encoding pendrin). The SLC26A4 enhancers and promoters described herein can be used to induce expression of the expression product in SLC26A4-expressing cells while reducing or eliminating off-target expression in non-SLC26A4-expressing cells (e.g., cochlear hair cells). The SLC26A4 enhancers can also be used to increase the expression level of the expression product in SLC26A4-expressing cells and to increase the number of SLC26A4-expressing cells in which the expression product is expressed. The compositions and methods described herein can, thus, be used to express an expression product (e.g., a protein, such as pendrin or another protein that is endogenously expressed in SLC26A4-expressing cells, or an RNA molecule, such as an inhibitory RNA molecule) in SLC26A4-expressing cells (e.g., interdental cells, spiral prominence cells, root cells, or vestibular supporting cells) to treat subjects having or at risk of developing hearing loss (e.g., sensorineural hearing loss) or deafness (e.g., pendrin-related hearing loss), subjects having or at risk of developing vestibular dysfunction (e.g., pendrin-related vestibular dysfunction or vestibular dysfunction associated with loss of vestibular hair cells), or subjects having Meniere's disease. The discovery of SLC26A4 enhancers and promoters that can improve cell type-specific expression can improve the safety and efficacy of gene therapy by reducing toxicity resulting from off-target expression.

The compositions and methods described herein can include an SLC26A4 enhancer listed in Table 2 (e.g., SEQ ID NO: 2 or SEQ ID NO: 3) or a variant thereof, such as a polynucleotide sequence that has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or SEQ ID NO: 3. In some embodiments, an SLC26A4 enhancer for use in the compositions and methods described herein has the sequence of SEQ ID NO: 2 or SEQ ID NO: 3. In some embodiments, the compositions described herein contain two or more (e.g., 2, 3, 4, 5, 6, 7, 8, 9, 10 or more) SLC26A4 enhancers, which can have the same sequence (e.g., multiple copies of the same SLC26A4 enhancer) or different sequences (e.g., one or more copies of each of the SLC26A4 enhancers listed in Table 2). In some embodiments, the compositions described herein contain one of each of the SLC26A4 enhancers listed in Table 2 (e.g., a single copy of SEQ ID NO: 2 and a single copy of SEQ ID NO: 3). In some embodiments, the compositions described herein contain multiple copies of SEQ ID NO: 2 (e.g., 2, 3, 4, 5, 6, 7, 8, 9, 10, or more copies) or multiple copies of SEQ ID NO: 3 (e.g., 2, 3, 4, 5, 6, 7, 8, 9, 10, or more copies). In some embodiments, the compositions described herein contain a single copy of SEQ NO: 2 and multiple copies of SEQ ID NO: 3 (e.g., 2, 3, 4, 5, 6, 7, 8, 9, 10, or more copies), or a single copy of SEQ ID NO: 3 and multiple copies of SEQ ID NO: 2 (e.g., 2, 3, 4, 5, 6, 7, 8, 9, 10, or more copies). In some embodiments, the compositions described herein contain multiple copies of both SEQ ID NO: 2 and SEQ ID NO: 3 (e.g., 2, 3, 4, 5, 6, 7, 8, 9, 10, or more copies of each enhancer). In embodiments in which a composition contains two or more enhancers (e.g., one or more copies of each of SEQ ID NO: 2 and SEQ ID NO: 3) the enhancers can be included in any order and may be positioned directly next to one another (e.g., joined without any intervening sequence between the enhancer sequences, e.g., the 3′ end of a first enhancer is positioned directly before the 5′ end of a second enhancer) or may be joined by a nucleic acid linker (e.g., a nucleic acid linker may be positioned between each enhancer sequence included in the composition or between at least two of the enhancer sequences in the composition).

Exemplary SLC26A4 enhancer sequences are provided in Table 2.

TABLE 2
SLC26A4 enhancer sequences

| SEQ | | |
|---|---|---|
| ID NO: | Enhancer | Enhancer sequence |
| 2 | Murine Pendrin E2 | TAAGTAGAATACGTGGTTAGAAAATCCAGCGCTAAGTAGGGTGAC |
| | enhancer (390 bp) | AAGCAAGGAACTGTTAAAGCCAGTGGAGTTGGGTTTTTTTTTTTTT |
| | | TCCAATTTTTTCCTCTTCTTTTATTTCAGACAATAATTGCTACTGCC |
| | | ATTTCCTATGGGGCCAACTTGGAAAAGAACTACAATGCTGGCATT |
| | | GTTAAGTCCATCCCAAGTGGGTGAGTGTGGCCTTCCTTCCAGATG |
| | | GCACTAATAAACTAGGGCAGCACTTTGTTTAAATGGAGCTTTTTAT |
| | | TACTGGTTTTGCTTTACTATTTATTGATAGTCTTACTTGCATGTGAT |
| | | TTTGGGGAAATGTCATGTGCACTACACATCAGACAAACAGCGCAT |
| | | GTGGGGACCAAGTTCAGGCCATTT |
| 3 | Murine Pendrin E6 | CAGTCAGGACTTCTGTGAGTCCACGGCCTTTGTCTGTAAGCTCTC |
| | enhancer (317 bp) | CGCTGACTCAGGGAAGGAAGTGGTGAGAAAGGCTGGAAGGTGGA |
| | | CAGGAGCCAGATAATGAAGGGCCTCACACACTGCATTAAGGAGCT |
| | | TGGATTTTAATCTCATCTGGTAAGAGATAAAGGCAAAGTGGTGGCT |
| | | TACATTACCTGGCTAATCCAATCAGCTGACCTAGGTACCTAGAAAT |
| | | AGATTCAGAGGAAATTAAAGTAGAGATGAATGAAGCTTTTGTTTTT |
| | | GTTTCTTTTTTTTTTCTCCCAAGGAGAGAGTAAGAGATGAAAGAA |

The SLC26A4 enhancer sequences described herein can be included in a nucleic acid vector and operably linked to a promoter (e.g., an SLC26A4 promoter, such as the promoter of SEQ ID NO: 1 or SEQ ID NO: 17), which can itself be operably linked to a polynucleotide encoding an expression product (e.g., a polynucleotide encoding a protein of interest, such as pendrin, or an RNA molecule, such as an inhibitory RNA) to express the expression product specifically in SLC26A4-expressing cells (e.g., in SLC26A4-expressing inner ear cells, such as interdental cells, root cells, spiral prominence cells, or vestibular supporting cells). According to the methods described herein, a subject can be administered a composition containing one or more of the foregoing polynucleotides (e.g., an SLC26A4 enhancer, e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or SEQ ID NO: 3) operably linked to a promoter that is operably linked to a polynucleotide encoding an expression product. The one or more SLC26A4 enhancers can be positioned 5′ of the promoter or 3′ of the promoter (e.g., 5′ of the promoter or 3′ of the coding sequence of the expression product).

The compositions and methods described herein can also include an SLC26A4 promoter. In some embodiments, the SLC26A4 promoter is operably linked to one or more of the foregoing enhancer sequences (e.g., SEQ ID NO: 2, SEQ ID NO: 3, or both). The SLC26A4 promoter can be operably linked to a polynucleotide encoding an expression product (e.g., a polynucleotide encoding pendrin, a polynucleotide encoding a protein or RNA molecule that is endogenously expressed in an SLC26A4-expressing cell, or a polynucleotide encoding an inhibitory RNA molecule). A nucleic acid vector containing an SLC26A4 promoter operably linked to a polynucleotide encoding pendrin can be used for the treatment of pendrin-related hearing loss (e.g., hearing loss associated with DFNB4 or Pendred syndrome), pendrin-related vestibular dysfunction (e.g., vestibular dysfunction associated with DFNB4 or Pendred syndrome), or for the treatment of Meniere's disease. Exemplary SLC26A4 promoters are provided in Table 3 below. In some embodiments, the SLC26A4 promoter for use in the compositions and methods described herein has at least 85% sequence identity (e.g., 85%, 86%, 87%, 88%, 89%, 90%, 91%, 92%, 93%, 94%, 95%, 96%, 97%, 98%, 99%, or 100% sequence identity) to SEQ ID NO: 1. In some embodiments, the SLC26A4 promoter for use in the compositions and methods described herein has the sequence of SEQ ID NO: 1. In some embodiments, the SLC26A4 promoter for use in the compositions and methods described herein has at least 85% sequence identity (e.g., 85%, 86%, 87%, 88%, 89%, 90%, 91%, 92%, 93%, 94%, 95%, 96%, 97%, 98%, 99%, or 100% sequence identity) to SEQ ID NO: 17. In some embodiments, the SLC26A4 promoter for use in the compositions and methods described herein has the sequence of SEQ ID NO: 17.

TABLE 3
SLC26A4 promoter and enhancer-promoter sequences

| SEQ ID | Description | |
|---|---|---|
| NO: | of Promoter | Promoter Sequence |
| 1 | Murine | AGCGTCAAAGAAGCAGGTGAGAAAGGGGCCACCTATACTCCTGTGAA |
| | SLC26A4 | TTAGACACATGCACAAAAGTGAAAAAGAATTTGTGTCTATAATCAGTGA |
| | promoter, | GTGCACTGTAATCTCTTCATTATATCCGCTGACTTTTCAGAGTTCCAAA |
| | (1111 bp) | ACTTTACTGCTATGCAAACCACATTAACTTCAATCCTTCTGCCCATTTG |
| | | GTGTAAGCATGCATGGACCAGGTTGTGGATTCTGACAGCCAAGGAAC |
| | | CTGGGTGCTTCAACAGGGAAGCTGTTCTCATAGCCATCGTGGTGTCCC |
| | | TGTGAGACAACCATTAAGAGTCATGGCTCTCTGGACTTGAGAAAGGGA |
| | | AAGAAAACAGGAGGGTAAGATTCGTGGGGAAAATCCTGTGTCCTTCCC |
| | | TTCCACCAAGGTGTCTCTGTTGCTCCATAAATAAAACGTCCCACCGCTT |
| | | TGAGTGAGAGCTATAAAGGCAGCAGAAAAACTGTCCCCAGGCATTCTG |
| | | GTCGGGCAGAACTCCGGAGCCCAAACAGGTGAGTTTATCACAGAAGA |
| | | GATGCAGTCGGCTGCACCCTGCCCGCTCTGTCGTAACTCTCTAGGGA |
| | | AGGTACTGGTCAGGCGCCGCGGGGGGGGTGGGCAGGACAGACACCA |
| | | GAAAAAGCGAGACAAGAGGAAGCTAAGGGTCAGACCGGGAAACTCGC |
| | | ACCTGTCCTTAAATGGGACTGCCCTGCTGCAGCCCAGAGTGTGCGTG |
| | | GAGCTGTGTCTCCGATCGAGTGAGGGGAGTACCGGACCCTTTCCCTG |
| | | TCGCACAGGCCCCAGTCCGATTCCGGGGAGATTTCAGGACGCGGACA |
| | | GCGCCCTGAATTGCCCAGGGAGGCCACCCAGCCCAACACCCAAGCC |
| | | CTCAAAGTGAGGCTCCCTCCCAGGACAGCGGGGATTCCCGGGTATCT |
| | | GGTACGGGAGACTGCAGTCCTCGGGTACCGTCCTGCAGGTGGCCGG |
| | | CCGCAGGGACAGTTTGTTGCCAGCGCCGAGGACTGCGGGACGCGGA |
| | | CCGGCTTTCCGAAGTCGCTGCCACAGGTGGCACCCGAGGCTGCTCCC |
| | | AGGTGCCCGCCGCGGGCCGTTTGCTCGCTCCGCGTTTCCGAACGTCA |
| | | TCCCTCGTCGCATCCCCTCCAG |
| 12 | Human | CTGCCTTCTGAGAGCGCTATAAAGGCAGCGGAAGGGTAGTCCGCGGG |
| | SLC26A4 | GCATTCCGGGGGG |
| | promoter 1 | |
| 13 | Human | CTCTAGGCGGGCTCTGCTCTTCTTTAAGGAGTCCCACAGGGCCTGGC |
| | SLC26A4 | CCGCCCCTGACCT |
| | promoter 2 | |
| 14 | Human | TAAAGAGTTGTGAGTTGTGTAGGTGAGTTGCCATGGAGCTACAAATAT |
| | SLC26A4 | GAGTTGATATTCTGAAATCCTAGACAGCCATCTCCAAGGTTAAGAAAAA |
| | enhancer- | TCCTTATGCACTCACTTGCAAAGATATCCACAGCATGCTCTTAATGGAG |
| | promoter | AAAAACAAAGCCTTAGATCAAATATGTAAAGTAATTTTTAGTTTTTTGAA |
| | (2000 bp) | AAGGTATGTTTGGGCTATAGATAAATCTGTTCAAAAAACATGAGAGAAG |
| | | ATAATAATGGTTGAAAGGAGACACAGTGCTTGCCCTCAAGAAGTTTTT |
| | | GTCTAGTGAGGGAGAGAGAACTTGTATGTAAATAAAATTGTGTTACTAA |
| | | GGTAGATAGTGAGAAGTAACTTAAGAGAGGATCAGATAAGGTATTAAG |
| | | AGAATACAGAAAAGGGTCTGGATTAATTCTGAACAGCATCAAAGAATG |
| | | TTCTTGCAAGAGATAGTGTTTTCACCAGATCTTGAAGGTATGGATGAG |
| | | GGTATACAGAGTGAGTATATTCAGATTCTACTTTAAAACAAATACTTTC |
| | | CTCTGTTGTAGTGGAGTTGAGCTATACATCCAACAATAATGAAAAAATA |
| | | CACGCATATATACATATATGGAGAGAGATACATATTTTAGTACATGTAG |
| | | CAATTGATTAATAAATGTACAGTTTAAGTCGCATGCAAAACCTTGGAGT |
| | | GATAGCAAACTTCATTGTAGGATGTTTAGCAGCATCTCTGGTCTCTACT |
| | | CACTAGATCCCAATAGCATCTCCCTAGGTGTGACAACCAAAAATGTCT |
| | | CCAGGCATTGACCTCTGGAGGCAAAAAAAGCCCTTTATTAAGAACCAG |
| | | TGGTATACATAAGTAAAACATACACAAGAGATTCCTCCCCTCTTCTCTG |
| | | TATGTGAATAAAAATTGCAAAGTTCATGACCTGGATTTTCCTTTTAGGTT |
| | | TCTTCTTTAGTGGTTCTTAACTTCATTGGGTGAAGTAAGCCTTTGAAGA |
| | | TCTGTTGAAAGCTGTTGACTCATTCACTTCTCAGGAAAACGCACATGCT |
| | | GACTACCATTTCAGAGAATTTGCATCAGGGTTCTCTGGGGAGGAGTTC |
| | | TGAGTTCTGTTTCCAGGAGCTCGTAGAATTGTCATGGTCTGCATATGC |
| | | AAGGCAGGTGGATTACGGAAGGTTGATGTACAGAGGTCTGTATTTTGG |
| | | AGCCTCTTCTGTATTTACTTCAGAACACTAACAATCAGGCGAGAATGTT |
| | | CTGGTTTATCAAACCCTTCCTTCTGCCTTTCATCTTAACCATGCATTAG |
| | | TTTTAACAAAGTTCATCCCAACAGAAGACAAAACACTGATGAGGTAGG |
| | | ATAGCTCCAGCTCCTCCTCCCTCTCTTCTAGTCTTGATTTCCATGTAGT |
| | | CCAGTTTATTCCTTCCCTGATTGTCCAGGAGAATGAGAAAAAGAAAAAA |
| | | CAGAGTCTAGTGGGTAAGAAAGGGCCACCTGGACGGCTTGATTTGGA |
| | | TTGTGAAATAAAACACACACACATGCACACGTAGAATAAGTGGCTAAAA |
| | | TCTGAGTAAATCGTGAACTCTCTGTATCCTCCACCCATTGAATACTCCT |
| | | AAAAGACTTTCTAGAAATTCAAGGACTTATTAATATAGAAACCTGGCCA |
| | | TTGTTCCTCTTCTCCTCCCCATGTGGTATGAGAGCACCTGTGGCAGGC |
| | | TCCCAGAGACCACGGACCTCTTCCTCTAGGGGGCTCTGCTCTTCTTT |
| | | AAGGAGTCCCACAGGGCCTGGCCCGCCCCTGACCTCGCAACCCTTGA |
| | | GATTAGTAACGGGATGAGTGAGGATCCGGGTGGCCCCTGCGTGGCAG |
| | | CCAGTAAGAGTCTCAGCCTTCCCGGTTCGGGAAAGGGGAAGAATGCA |
| | | GGAGGGGTAGGATTTCTTTCCTGATAGGATCGGTTGGGAAAGACCGC |
| | | AGCCTGTGTGTGTCTTTCCCTTCGACCAAGGTGTCTGTTGCTCCGTAA |
| | | ATAAAACGTCCCACTGCCTTCTGAGAGCGCTATAAAGGCAGCGGAAG |
| | | GGTAGTCCGCGGGGC |
| 15 | Human | GGCTGCTCGGAAAACAGGACGAGGGGAGAGACTTGCTCAATAAGCTG |
| | SLC26A4 | AAAGTTCTGCCCCCGAGAGGGCTGCGACAGCTGCTGGAATGTGCCTG |
| | enhancer- | CAGCGTCCGCCTCTTGGGGACCCGCGGAGCGCGCCCTGACGGTTCC |
| | promoter | ACGCCTGGCCCGGGGGTCTGCACCTCTCCTCCAGTGCGCACCTGGA |
| | (1400 bp) | GCTGCGTCCCGGGTCAGGTGCGGGGAGGGAGGGAATCTCAGTGTCC |
| | | CCTTCCAGCCTTGCAAGCGCCTTTGGCCCCTGCCCCAGCCCCTCGGT |
| | | TTGGGGGAGATTTCAGAACGCGGACAGCGCCCTGGCTGCGGGCCATA |
| | | GGGGACTGGGTGGAACTCGGGAAGCCCCCAGAGCAGGGGCTTACTC |
| | | GCTTCAAGTTTGGGGAACCCCGGGCAGCGGGTGCAGGCCACGAGAC |
| | | CCGAAGGTTCTCAGGTGCCCCCCTGCAGGCTGGCCGTGCGCGCCGT |
| | | GGGGCGCTTGTCGCGAGCGCCGAGGGCTGCAGGACGCGGACCAGAC |
| | | TCGCGGTGCAGGGGGGCCTGGCTGCAGCTAACAGGTGATCCCGTTCT |
| | | TTCTGTTCCTCGCTCTTCCCCTCCGATCGTCCTCGCTTACCGCGTGTC |
| | | CTCCCTCCTCGCTGTCCTCTGGCTCGCAGGTCATGGCAGCGCCAGGC |
| | | GGCAGGTCGGAGCCGCCGCAGCTCCCCGAGTACAGCTGCAGCTACA |
| | | TGGTGTCGCGGCCGGTCTACAGCGAGCTCGCTTTCCAGCAACAGCAC |
| | | GAGCGGCGCCTGCAGGAGCGCAAGACGCTGCGGGAGAGCCTGGCCA |
| | | AGTGCTGCAGGTAGCGGCCGCGCGGGCCTGCGTAGAGAGAAGCGGA |
| | | GCGGGGCGTCCACGCCTTGGGGAGGGAAGGGCGTCCCCAGCGGGC |
| | | GAGAGTGGGGTGCGGGCGGCGGAGCCCCTGGGCGCCAGCTGCTTCT |
| | | CCCAGAGGCCCGACTTTCGGTCTCCGGTCCTCCACGCCGCCCTTCTG |
| | | GTGGGAGGGTGGCTCCATCAGTCTCGGGCCCGAAATGAACTTACCTG |
| | | GGAAACTCGCCTTTGGGGAGAGTGGGTTCTAGGAGCCCCGTCTCTCT |
| | | TTTTCCTCTCTGAAGGAAACTTGGAGTGCCTCTTGGGGTACAGTGGGT |
| | | CCCTGTTGCCTTCTTGGGAGCTTGTTTAAATGAAATGAATAGGGAAAC |
| | | CCAGCTCTTGACCAGGAGGAGTCCTTGAAACACTCAAGCTAAGTAGGC |
| | | GGGCTACCATTCAGTTAGAGACCAGGATGCAAGCTAGAACCCAGGGG |
| | | AGCGCGGGGTGTGCCAAGTACTTCATCAGCAGGCTGTGGGACCCCTG |
| | | GGGAAAGCCACCCTCAGTCTCTAAACCCAAACATGCCGTAACTAGATG |
| | | TCACAAACATAAAGAAATTAGAGTTTCTAAAACCTTTCATTATAG |
| 16 | Human | CGGAAGGTTGATGTACAGAGGTCTGTATTTTGGAGCCTCTTCTGTATTT |
| | SLC26A4 | ACTTCAGAACACTAACAATCAGGCGAGAATGTTCTGGTTTATCAAACCC |
| | enhancer- | TTCCTTCTGCCTTTCATCTTAACCATGCATTAGTTTTAACAAAGTTCATC |
| | promoter | CCAACAGAAGACAAAACACTGATGAGGTAGGATAGCTCCAGCTCCTCC |
| | (900 bp) | TCCCTCTCTTCTAGTCTTGATTTCCATGTAGTCCAGTTTATTCCTTCCCT |
| | | GATTGTCCAGGAGAATGAGAAAAAGAAAAAACAGAGTCTAGTGGGTAA |
| | | GAAAGGGCCACCTGGACGGCTTGATTTGGATTGTGAAATAAAACACAC |
| | | ACACATGCACACGTAGAATAAGTGGCTAAAATCTGAGTAAATCGTGAA |
| | | CTCTCTGTATCCTCCACCCATTGAATACTCCTAAAAGACTTTCTAGAAA |
| | | TTCAAGGACTTATTAATATAGAAACCTGGCCATTGTTCCTCTTCTCCTC |
| | | CCCATGTGGTATGAGAGCACCTGTGGCAGGCTCCCAGAGACCACGGA |
| | | CCTCTTCCTCTAGGCGGGCTCTGCTCTTCTTTAAGGAGTCCCACAGGG |
| | | CCTGGCCCGCCCCTGACCTCGCAACCCTTGAGATTAGTAACGGGATG |
| | | AGTGAGGATCCGGGTGGCCCCTGCGTGGCAGCCAGTAAGAGTCTCAG |
| | | CCTTCCCGGTTCGGGAAAGGGGAAGAATGCAGGAGGGGTAGGATTTC |
| | | TTTCCTGATAGGATCGGTTGGGAAAGACCGCAGCCTGTGTGTGTCTTT |
| | | CCCTTCGACCAAGGTGTCTGTTGCTCCGTAAATAAAACGTCCCACTGC |
| | | CTTCTGAGAGCGCTATAAAGGCAGCGGAAGGGTAGTCCGCGGGGCAT |
| | | TCCGGGGGGGGCGCGAGCAGAGACAGGTGAGTT |
| 17 | Murine | GAAAGAAAACAGGAGGGTAAGATTCGTGGGGAAAATCCTGTGTCCTTC |
| | SLC26A4 | CCTTCCACCAAGGTGTCTCTGTTGCTCCATAAATAAAACGTCCCACCG |
| | minimal | CTTTGAGTGAGAGCTATAAAGGCAGCAGAAAAACTGTCCCCAGGCATT |
| | promoter | CTGGTCGGGCAGAACTCCGGAGCCCAAACAGGTGAGTTTATCACAGA |
| | (640 bp) | AGAGATGCAGTCGGCTGCACCCTGCCCGCTCTGTCGTAACTCTCTAG |
| | | GGAAGGTACTGGTCAGGCGCCGCGGGGGGGGTGGGCAGGACAGACA |
| | | CCAGAAAAAGCGAGACAAGAGGAAGCTAAGGGTCAGACCGGGAAACT |
| | | CGCACCTGTCCTTAAATGGGACTGCCCTGCTGCAGCCCAGAGTGTGC |
| | | GTGGAGCTGTGTCTCCGATCGAGTGAGGGGAGTACCGGACCCTTTCC |
| | | CTGTCGCACAGGCCCCAGTCCGATTCCGGGGAGATTTCAGGACGCGG |
| | | ACAGCGCCCTGAATTGCCCAGGGAGGCCACCCAGCCCAACACCCAAG |
| | | CCCTCAAAGTGAGGCTCCCTCCCAGGACAGCGGGGATTCCCGGGTAT |
| | | CTGGTACGGGAGACTGCAGTCCTCGGGTACCGTCCTGCAGGTGGCC |
| | | GGCCGCAGGGACAGTTTGTTGCCAGCGC |
| 18 | Murine E2 | TAAGTAGAATACGTGGTTAGAAAATCCAGCGCTAAGTAGGGTGACAAG |
| | enhancer- | CAAGGAACTGTTAAAGCCAGTGGAGTTGGGTTTTTTTTTTTTTTCCAAT |
| | murine E6 | TTTTTCCTCTTCTTTTATTTCAGACAATAATTGCTACTGCCATTTCCTAT |
| | enhancer- | GGGGCCAACTTGGAAAAGAACTACAATGCTGGCATTGTTAAGTCCATC |
| | murine | CCAAGTGGGTGAGTGTGGCCTTCCTTCCAGATGGCACTAATAAACTAG |
| | SLC26A4 | GGCAGCACTTTGTTTAAATGGAGCTTTTTATTACTGGTTTTGCTTTACTA |
| | minimal | TTTATTGATAGTCTTACTTGCATGTGATTTTGGGGAAATGTCATGTGCA |
| | promoter | CTACACATCAGACAAACAGCGCATGTGGGGACCAAGTTCAGGCCATTT |
| | (1347 bp) | CAGTCAGGACTTCTGTGAGTCCACGGCCTTTGTCTGTAAGCTCTCCGC |
| | | TGACTCAGGGAAGGAAGTGGTGAGAAAGGCTGGAAGGTGGACAGGA |
| | | GCCAGATAATGAAGGGCCTCACACACTGCATTAAGGAGCTTGGATTTT |
| | | AATCTCATCTGGTAAGAGATAAAGGCAAAGTGGTGGCTTACATTACCT |
| | | GGCTAATCCAATCAGCTGACCTAGGTACCTAGAAATAGATTCAGAGGA |
| | | AATTAAAGTAGAGATGAATGAAGCTTTTGTTTTTGTTTCTTTTTTTTTTCT |
| | | CCCAAGGAGAGAGTAAGAGATGAAAGAAGAAAGAAAACAGGAGGGTA |
| | | AGATTCGTGGGGAAAATCCTGTGTCCTTCCCTTCCACCAAGGTGTCTC |
| | | TGTTGCTCCATAAATAAAACGTCCCACCGCTTTGAGTGAGAGCTATAAA |
| | | GGCAGCAGAAAAACTGTCCCCAGGCATTCTGGTCGGGCAGAACTCCG |
| | | GAGCCCAAACAGGTGAGTTTATCACAGAAGAGATGCAGTCGGCTGCA |
| | | CCCTGCCCGCTCTGTCGTAACTCTCTAGGGAAGGTACTGGTCAGGCG |
| | | CCGCGGGGGGGGTGGGCAGGACAGACACCAGAAAAAGCGAGACAAG |
| | | AGGAAGCTAAGGGTCAGACCGGGAAACTCGCACCTGTCCTTAAATGG |
| | | GACTGCCCTGCTGCAGCCCAGAGTGTGCGTGGAGCTGTGTCTCCGAT |
| | | CGAGTGAGGGGAGTACCGGACCCTTTCCCTGTCGCACAGGCCCCAGT |
| | | CCGATTCCGGGGAGATTTCAGGACGCGGACAGCGCCCTGAATTGCCC |
| | | AGGGAGGCCACCCAGCCCAACACCCAAGCCCTCAAAGTGAGGCTCCC |
| | | TCCCAGGACAGCGGGGATTCCCGGGTATCTGGTACGGGAGACTGCAG |
| | | TCCTCGGGTACCGTCCTGCAGGTGGCCGGCCGCAGGGACAGTTTGTT |
| | | GCCAGCGC |

In some embodiments, a polynucleotide encoding wild-type pendrin, or a variant thereof, such as a polynucleotide sequence that encodes a protein having at least 85% sequence identity (e.g., 85%, 86%, 87%, 88%, 89%, 90%, 91%, 92%, 93%, 94%, 95%, 96%, 97%, 98%, 99%, or more, sequence identity) to the amino acid sequence of wild-type mammalian (e.g., human or mouse) pendrin (e.g., SEQ ID NO: 4 or SEQ ID NO: 5) is operably linked to an SLC26A4 promoter described herein (e.g., a promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or a promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17), or is operably linked to a promoter (e.g., an SLC26A4 promoter, such as an SLC26A4 promoter or enhancer-promoter provided in Table 3, a minimal promoter, a core promoter, or a constitutive promoter) that is operably linked to one or more SLC26A4 enhancers described herein (e.g., one or more copies of an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 and/or SEQ ID NO: 3). In some embodiments, the polynucleotide encoding wild-type pendrin, or a variant thereof is operably linked to a polynucleotide that contains, in 5′ to 3′ order, an enhancer that has the sequence of SEQ ID NO: 2, an enhancer that has the sequence of SEQ ID NO: 3, and a promoter that has the sequence of SEQ ID NO: 17. In some more specific embodiments, the polynucleotide encoding wild-type pendrin, or a variant thereof is operably linked to the sequence of SEQ ID NO: 18. In some embodiments, the polynucleotide sequence encoding a pendrin protein encodes an amino acid sequence that contains one or more conservative amino acid substitutions relative to SEQ ID NO: 4 or SEQ ID NO: 5 (e.g., 1, 2, 3, 4, 5, 6, 7, 8, 9, 10, 11, 12, 13, 14, 15, 16, 17, 18, 19, 20 or more conservative amino acid substitutions), provided that the pendrin analog encoded retains the therapeutic function of wild-type pendrin (e.g., the ability to transport negatively charged ions, such as chloride, iodide, and bicarbonate, across cell membranes). No more than 10% of the amino acids in the pendrin protein may be replaced with conservative amino acid substitutions. In some embodiments, the polynucleotide sequence that encodes pendrin is any polynucleotide sequence that, by redundancy of the genetic code, encodes SEQ ID NO: 4 or SEQ ID NO: 5. The polynucleotide sequence that encodes pendrin can be partially or fully codon-optimized for expression (e.g., in human SLC26A4-expressing inner ear cells). The pendrin protein may also be encoded by a polynucleotide having single nucleotide polymorphisms (SNPs) that have been found to be non-pathogenic in human subjects (e.g., SNPs that do not result in hearing loss). Human pendrin may be encoded by a polynucleotide having the sequence of SEQ ID NO: 6. Murine pendrin may be encoded by a polynucleotide having the sequence of SEQ ID NO: 7. The pendrin protein may be a human pendrin protein or may be a homolog of the human pendrin protein from another mammalian species (e.g., mouse, rat, cow, horse, goat, sheep, donkey, cat, dog, rabbit, guinea pig, or other mammal). Exemplary pendrin amino acid and polynucleotide sequences are provided in Table 4, below. A nucleic acid vector (e.g., an AAV vector) containing an SLC26A4 enhancer described herein operably linked to a promoter (e.g., an SLC26A4 promoter, such as an SLC26A4 promoter or enhancer-promoter provided in Table 3, a minimal promoter, a core promoter, or a constitutive promoter) that is operably inked to a polynucleotide encoding pendrin, or a nucleic acid vector (e.g., an AAV vector) containing an SLC26A4 promoter described herein (e.g., SEQ ID NO: 1 or SEQ ID NO: 17) operably inked to a polynucleotide encoding pendrin, can be administered to a subject to treat, reduce, or prevent pendrin-related hearing loss, such as hearing loss in a subject having DFNB4 or Pendred syndrome, or pendrin-related vestibular dysfunction, such as vestibular dysfunction (e.g., imbalance or loss of balance, dizziness, or vertigo) associated with DFNB4 or Pendred syndrome, or can be administered to a subject to treat Meniere's disease (e.g., hearing loss, tinnitus, or vestibular dysfunction associated with Meniere's disease). Such nucleic acid vectors may also be administered to a subject to treat vestibular dysfunction (e.g., vertigo, dizziness, or imbalance or loss of balance) associated with damage to or loss of vestibular hair cells (damage to or loss of vestibular hair cells related to head trauma, disease or infection, ototoxic drugs, or aging, e.g., age-related vestibular dysfunction, ototoxic-drug induced vestibular dysfunction, disease or infection-related vestibular dysfunction, or head trauma-related vestibular dysfunction).

TABLE 4
Pendrin sequences

| SEQ ID | Description of | |
|---|---|---|
| NO: | sequence | Sequence |
| 4 | Human Pendrin | MAAPGGRSEPPQLPEYSCSYMVSRPVYSELAFQQQHERRLQERK |
| | amino acid | TLRESLAKCCSCSRKRAFGVLKTLVPILEWLPKYRVKEWLLSDVISG |
| | sequence (UniProt | VSTGLVATLQGMAYALLAAVPVGYGLYSAFFPILTYFIFGTSRHISVG |
| | O43511) | PFPVVSLMVGSVVLSMAPDEHFLVSSSNGTVLNTTMIDTAARDTAR |
| | | VLIASALTLLVGIIQLIFGGLQIGFIVRYLADPLVGGFTTAAAFQVLVSQ |
| | | LKIVLNVSTKNYNGVLSIIYTLVEIFQNIGDTNLADFTAGLLTIVVCMA |
| | | VKELNDRFRHKIPVPIPIEVIVTIIATAISYGANLEKNYNAGIVKSIPRG |
| | | FLPPELPPVSLFSEMLAASFSIAVVAYAIAVSVGKVYATKYDYTIDGN |
| | | QEFIAFGISNIFSGFFSCFVATTALSRTAVQESTGGKTQVAGIISAAIV |
| | | MIAILALGKLLEPLQKSVLAAVVIANLKGMFMQLCDIPRLWRQNKIDA |
| | | VIWVFTCIVSIILGLDLGLLAGLIFGLLTVVLRVQFPSWNGLGSIPSTDI |
| | | YKSTKNYKNIEEPQGVKILRFSSPIFYGNVDGFKKCIKSTVGFDAIRV |
| | | YNKRLKALRKIQKLIKSGQLRATKNGIISDAVSTNNAFEPDEDIEDLE |
| | | ELDIPTKEIEIQVDWNSELPVKVNVPKVPIHSLVLDCGAISFLDVVGV |
| | | RSLRVIVKEFQRIDVNVYFASLQDYVIEKLEQCGFFDDNIRKDTFFLT |
| | | VHDAILYLQNQVKSQEGQGSILETITLIQDCKDTLELIETELTEEELDV |
| | | QDEAMRTLAS |
| 5 | Mouse Pendrin | MAARGGRSEPPQLAEYSCSYTVSRPVYSELAFQQQRERRLPERRT |
| | amino acid | LRDSLARSCSCSRKRAFGVVKTLLPILDWLPKYRVKEWLLSDIISGV |
| | sequence (UniProt | STGLVGTLQGMAYALLAAVPVQFGLYSAFFPILTYFVFGTSRHISVG |
| | Q9R155) | PFPVVSLMVGSVVLSMAPDDHFLVPSGNGSALNSTTLDTGTRDAA |
| | | RVLLASTLTLLVGIIQLVFGGLQIGFIVRYLADPLVGGFTTAAAFQVLV |
| | | SQLKIVLNVSTKNYNGILSIIYTLIEIFQNIGDTNIADFIAGLLTIIVCMAV |
| | | KELNDRFKHRIPVPIPIEVIVTIIATAISYGANLEKNYNAGIVKSIPSGFL |
| | | PPVLPSVGLFSDMLAASFSIAVVAYAIAVSVGKVYATKHDYVIDGNQ |
| | | EFIAFGISNVFSGFFSCFVATTALSRTAVQESTGGKTQVAGLISAVIV |
| | | MVAIVALGRLLEPLQKSVLAAVVIANLKGMFMQVCDVPRLWKQNKT |
| | | DAVIWVFTCIMSIILGLDLGLLAGLLFALLTVVLRVQFPSWNGLGSVP |
| | | STDIYKSITHYKNLEEPEGVKILRFSSPIFYGNVDGFKKCINSTVGFD |
| | | AIRVYNKRLKALRRIQKLIKKGQLRATKNGIISDIGSSNNAFEPDEDV |
| | | EEPEELNIPTKEIEIQVDWNSELPVKVNVPKVPIHSLVLDCGAVSFLD |
| | | VVGVRSLRMIVKEFQRIDVNVYFALLQDDVLEKMEQCGFFDDNIRK |
| | | DRFFLTVHDAILHLQNQVKSREGQDSLLETVARIRDCKDPLDLMEA |
| | | EMNAEELDVQDEAMRRLAS |
| 6 | Nucleic acid | ATGGCAGCGCCAGGCGGCAGGTCGGAGCCGCCGCAGCTCCCC |
| | sequence encoding | GAGTACAGCTGCAGCTACATGGTGTCGCGGCCGGTCTACAGCG |
| | human Pendrin | AGCTCGCTTTCCAGCAACAGCACGAGCGGCGCCTGCAGGAGCG |
| | | CAAGACGCTGCGGGAGAGCCTGGCCAAGTGCTGCAGTTGTTCA |
| | | AGAAAGAGAGCCTTTGGTGTGCTAAAGACTCTTGTGCCCATCTT |
| | | GGAGTGGCTCCCCAAATACCGAGTCAAGGAATGGCTGCTTAGT |
| | | GACGTCATTTCGGGAGTTAGTACTGGGCTAGTGGCCACGCTGC |
| | | AAGGGATGGCATATGCCCTACTAGCTGCAGTTCCTGTCGGATAT |
| | | GGTCTCTACTCTGCTTTTTTCCCTATCCTGACATACTTTATCTTTG |
| | | GAACATCAAGACATATCTCAGTTGGACCTTTTCCAGTGGTGAGTT |
| | | TAATGGTGGGATCTGTTGTTCTGAGCATGGCCCCCGACGAACAC |
| | | TTTCTCGTATCCAGCAGCAATGGAACTGTATTAAATACTACTATG |
| | | ATAGACACTGCAGCTAGAGATACAGCTAGAGTCCTGATTGCCAG |
| | | TGCCCTGACTCTGCTGGTTGGAATTATACAGTTGATATTTGGTG |
| | | GCTTGCAGATTGGATTCATAGTGAGGTACTTGGCAGATCCTTTG |
| | | GTTGGTGGCTTCACAACAGCTGCTGCCTTCCAAGTGCTGGTCTC |
| | | ACAGCTAAAGATTGTCCTCAATGTTTCAACCAAAAACTACAATGG |
| | | AGTTCTCTCTATTATCTATACGCTGGTTGAGATTTTTCAAAATATT |
| | | GGTGATACCAATCTTGCTGATTTCACTGCTGGATTGCTCACCATT |
| | | GTCGTCTGTATGGCAGTTAAGGAATTAAATGATCGGTTTAGACA |
| | | CAAAATCCCAGTCCCTATTCCTATAGAAGTAATTGTGACGATAAT |
| | | TGCTACTGCCATTTCATATGGAGCCAACCTGGAAAAAAATTACAA |
| | | TGCTGGCATTGTTAAATCCATCCCAAGGGGGTTTTTGCCTCCTG |
| | | AACTTCCACCTGTGAGCTTGTTCTCGGAGATGCTGGCTGCATCA |
| | | TTTTCCATCGCTGTGGTGGCTTATGCTATTGCAGTGTCAGTAGG |
| | | AAAAGTATATGCCACCAAGTATGATTACACCATCGATGGGAACC |
| | | AGGAATTCATTGCCTTTGGGATCAGCAACATCTTCTCAGGATTCT |
| | | TCTCTTGTTTTGTGGCCACCACTGCTCTTTCCCGCACGGCCGTC |
| | | CAGGAGAGCACTGGAGGAAAGACACAGGTTGCTGGCATCATCT |
| | | CTGCTGCGATTGTGATGATCGCCATTCTTGCCCTGGGGAAGCTT |
| | | CTGGAACCCTTGCAGAAGTCGGTCTTGGCAGCTGTTGTAATTGC |
| | | CAACCTGAAAGGGATGTTTATGCAGCTGTGTGACATTCCTCGTC |
| | | TGTGGAGACAGAATAAGATTGATGCTGTTATCTGGGTGTTTACG |
| | | TGTATAGTGTCCATCATTCTGGGGCTGGATCTCGGTTTACTAGC |
| | | TGGCCTTATATTTGGACTGTTGACTGTGGTCCTGAGAGTTCAGTT |
| | | TCCTTCTTGGAATGGCCTTGGAAGCATCCCTAGCACAGATATCT |
| | | ACAAAAGTACCAAGAATTACAAAAACATTGAAGAACCTCAAGGA |
| | | GTGAAGATTCTTAGATTTTCCAGTCCTATTTTCTATGGCAATGTC |
| | | GATGGTTTTAAAAAATGTATCAAGTCCACAGTTGGATTTGATGCC |
| | | ATTAGAGTATATAATAAGAGGCTGAAAGCGCTGAGGAAAATACA |
| | | GAAACTAATAAAAAGTGGACAATTAAGAGCAACAAAGAATGGCA |
| | | TCATAAGTGATGCTGTTTCAACAAATAATGCTTTTGAGCCTGATG |
| | | AGGATATTGAAGATCTGGAGGAACTTGATATCCCAACCAAGGAA |
| | | ATAGAGATTCAAGTGGATTGGAACTCTGAGCTTCCAGTCAAAGT |
| | | GAACGTTCCCAAAGTGCCAATCCATAGCCTTGTGCTTGACTGTG |
| | | GAGCTATATCTTTCCTGGACGTTGTTGGAGTGAGATCACTGCGG |
| | | GTGATTGTCAAAGAATTCCAAAGAATTGATGTGAATGTGTATTTT |
| | | GCATCACTTCAAGATTATGTGATAGAAAAGCTGGAGCAATGCGG |
| | | GTTCTTTGACGACAACATTAGAAAGGACACATTCTTTTTGACGGT |
| | | CCATGATGCTATACTCTATCTACAGAACCAAGTGAAATCTCAAGA |
| | | GGGTCAAGGTTCCATTTTAGAAACGATCACTCTCATTCAGGATTG |
| | | TAAAGATACCCTTGAATTAATAGAAACAGAGCTGACGGAAGAAG |
| | | AACTTGATGTCCAGGATGAGGCTATGCGTACACTTGCATCC |
| 7 | Nucleic acid | ATGGCAGCGCGGGGCGGCAGGTCGGAGCCGCCGCAGCTCGCC |
| | sequence encoding | GAGTACAGCTGCAGTTACACGGTGTCGCGGCCGGTGTACAGCG |
| | mouse Pendrin | AGCTCGCCTTCCAGCAGCAGCGCGAGCGGCGCCTGCCTGAGC |
| | | GCAGGACGCTGCGGGACAGCCTGGCGCGGAGCTGCAGTTGCT |
| | | CAAGAAAGAGAGCCTTTGGTGTGGTAAAGACTCTCCTGCCCATT |
| | | CTGGACTGGCTCCCAAAATACCGAGTCAAGGAATGGCTCCTCAG |
| | | TGACATCATCTCCGGAGTTAGCACTGGGCTGGTGGGTACCCTG |
| | | CAAGGGATGGCTTATGCCCTGCTGGCAGCAGTACCTGTTCAGTT |
| | | CGGTCTCTACTCTGCCTTTTTCCCTATCCTGACGTATTTTGTGTT |
| | | CGGAACATCAAGACACATCTCAGTTGGCCCTTTCCCCGTGGTCA |
| | | GTTTAATGGTGGGATCTGTTGTTCTGAGCATGGCTCCAGATGAC |
| | | CACTTTCTTGTGCCCAGCGGTAACGGAAGTGCATTGAACTCGAC |
| | | CACGTTAGACACTGGAACCAGAGATGCGGCCCGAGTGTTGCTT |
| | | GCAAGCACACTCACTCTTCTAGTTGGAATCATACAGCTGGTGTTT |
| | | GGAGGTTTGCAGATTGGATTCATAGTGAGGTACTTGGCAGACCC |
| | | CTTGGTTGGCGGATTCACAACCGCTGCAGCCTTCCAAGTACTGG |
| | | TCTCACAGCTAAAGATCGTGCTCAATGTTTCAACCAAAAACTACA |
| | | ACGGCATCCTCTCCATTATCTACACACTAATTGAGATTTTTCAAA |
| | | ATATCGGTGACACCAATATTGCCGATTTCATCGCTGGGCTGCTG |
| | | ACCATCATCGTCTGTATGGCTGTTAAGGAACTAAATGATCGATTT |
| | | AAACACAGAATCCCGGTGCCCATTCCTATAGAAGTGATTGTGAC |
| | | AATAATTGCTACTGCCATTTCCTATGGGGCCAACTTGGAAAAGAA |
| | | CTACAATGCTGGCATTGTTAAGTCCATCCCAAGTGGGTTCTTGC |
| | | CTCCTGTCCTGCCATCTGTGGGCCTGTTTTCGGACATGTTGGCT |
| | | GCATCCTTTTCCATTGCTGTGGTGGCTTACGCTATTGCAGTGTCT |
| | | GTAGGAAAAGTCTACGCCACCAAGCATGACTATGTCATCGATGG |
| | | GAACCAGGAATTCATTGCCTTTGGGATAAGCAACGTCTTCTCTG |
| | | GATTTTTCTCCTGTTTTGTGGCTACCACTGCTCTGTCTCGAACGG |
| | | CTGTCCAGGAGAGCACCGGGGGGAAGACACAGGTGGCTGGCC |
| | | TCATCTCAGCTGTGATTGTGATGGTTGCCATCGTTGCCTTGGGG |
| | | AGGCTTCTGGAACCCTTGCAGAAGTCAGTCTTGGCGGCCGTTGT |
| | | CATTGCCAACCTGAAAGGGATGTTTATGCAGGTGTGTGACGTTC |
| | | CTCGTCTGTGGAAGCAGAATAAGACTGATGCTGTTATCTGGGTG |
| | | TTTACATGCATAATGTCCATCATTCTGGGGCTGGACCTCGGCTT |
| | | GCTAGCTGGCCTTTTATTTGCACTACTGACTGTGGTCCTGAGAG |
| | | TTCAGTTCCCTTCATGGAATGGCCTTGGAAGTGTCCCCAGCACA |
| | | GACATCTACAAAAGCATCACACATTATAAAAACCTTGAAGAGCCT |
| | | GAAGGGGTGAAGATCCTGAGATTTTCCAGTCCCATTTTTTACGG |
| | | CAATGTCGATGGTTTTAAAAAATGTATCAATTCAACGGTTGGATT |
| | | TGATGCCATTAGAGTATATAATAAGAGGCTGAAAGCACTGAGAA |
| | | GAATACAGAAACTCATCAAAAAAGGACAACTCAGGGCAACCAAG |
| | | AACGGGATCATAAGTGATATTGGCTCATCAAATAATGCCTTCGA |
| | | GCCTGATGAAGATGTGGAAGAGCCAGAGGAACTTAATATCCCAA |
| | | CCAAAGAAATTGAGATTCAAGTGGACTGGAACTCCGAACTCCCG |
| | | GTGAAAGTGAATGTCCCAAAGGTGCCAATCCACAGCCTGGTGCT |
| | | GGATTGCGGAGCTGTATCCTTCCTGGATGTGGTAGGAGTGAGG |
| | | TCATTGCGAATGATTGTCAAAGAATTTCAGAGAATTGATGTGAAT |
| | | GTGTACTTTGCTCTGCTTCAAGATGATGTGTTAGAAAAGATGGA |
| | | GCAGTGTGGGTTCTTTGATGACAACATTAGAAAGGACAGATTCT |
| | | TTCTGACGGTTCATGATGCAATCCTCCATCTGCAGAACCAGGTC |
| | | AAATCCAGAGAAGGCCAGGATTCCCTGCTAGAGACGGTCGCTC |
| | | GCATTCGGGACTGTAAAGACCCTCTTGATCTGATGGAGGCAGAG |
| | | ATGAATGCAGAAGAGCTCGATGTTCAGGATGAGGCCATGCGTA |
| | | GACTTGCTTCC |

### Expression of Exogenous Polynucleotides in Mammalian Cells

The compositions and methods described herein can be used to induce or increase the expression of exogenous polynucleotides (e.g., a gene that is endogenously expressed in SLC26A4-expressing cells, such as a polynucleotide encoding pendrin) specifically in SLC26A4-expressing cells (e.g., SLC26A4-expressing inner ear cells, such as interdental cells, root cells, spiral prominence cells, and vestibular supporting cells) by administering a nucleic acid vector that contains at least one SLC26A4 enhancer (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or SEQ ID NO: 3) operably linked to a promoter (e.g., an SLC26A4 promoter, such as an SLC26A4 promoter or enhancer-promoter provided in Table 3, a minimal promoter, a core promoter, or a constitutive promoter) that is operably linked to a polynucleotide that encodes an expression product (e.g., a protein of interest or an RNA molecule, such as an inhibitory RNA), or by administering a nucleic acid vector that contains an SLC26A4 promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17) operably linked to a polynucleotide that encodes an expression product (e.g., a protein of interest or an RNA molecule, such as an inhibitory RNA). In some embodiments, the nucleic acid vector contains, in 5′ to 3′ order, an enhancer that has the sequence of SEQ ID NO: 2, an enhancer that has the sequence of SEQ ID NO: 3, and a promoter that has the sequence of SEQ ID NO: 17. In some embodiments the nucleic acid vector contains the sequence of SEQ ID NO: 18. A wide array of methods has been established for the delivery of proteins to mammalian cells and for the stable expression of polynucleotides encoding proteins in mammalian cells.

The nucleic acid vectors (e.g., AAV vectors) described herein (e.g., a nucleic acid vector that contains at least one SLC26A4 enhancer (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or SEQ ID NO: 3) operably linked to a promoter (e.g., an SLC26A4 promoter, such as an SLC26A4 promoter or enhancer-promoter provided in Table 3, a minimal promoter, a core promoter, or a constitutive promoter) or a nucleic acid vector that contains an SLC26A4 promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17) can be used to express a polynucleotide in one or more SLC26A4-expressing cells (e.g., SLC26A4-expressing inner ear cells). In some embodiments, the nucleic acid vector contains, in 5′ to 3′ order, an enhancer that has the sequence of SEQ ID NO: 2, an enhancer that has the sequence of SEQ ID NO: 3, and a promoter that has the sequence of SEQ ID NO: 17. In some embodiments the nucleic acid vector contains the sequence of SEQ ID NO: 18. Exemplary polynucleotides that can be expressed using a nucleic acid vector described herein include polynucleotides encoding proteins that are expressed in healthy SLC26A4-expressing cells, such as pendrin and Atoh1, polynucleotides that correspond to a wild-type form of a gene that is endogenously expressed in a SLC26A4-expressing inner ear cell and mutated in a subject with hearing loss, deafness, tinnitus, or vestibular dysfunction, and other polynucleotides that can be expressed in SLC26A4-expressing inner ear cells to treat hearing loss, deafness, tinnitus, or vestibular dysfunction. The nucleic acid vectors described herein can also be used to express a short hairpin RNA (shRNA), an antisense oligonucleotide (ASO), a component of a gene editing system (e.g., a nuclease, such as a CRISPR Associated Protein 9 (Cas9), Transcription Activator-Like Effector Nuclease (TALEN), or Zinc Finger Nuclease (ZFN), or a guide RNA (gRNA)), or a microRNA (e.g., miR-183, miR-96, or miR-182) in SLC26A4-expressing cells (e.g., SLC26A4-expressing inner ear cells, such as interdental cells, root cells, spiral prominence cells, and vestibular supporting cells).

In some embodiments, a polynucleotide encoding wild-type Atoh1, or a variant thereof, such as a polynucleotide sequence that encodes a protein having at least 85% sequence identity (e.g., 85%, 86%, 87%, 88%, 89%, 90%, 91%, 92%, 93%, 94%, 95%, 96%, 97%, 98%, 99%, or more, sequence identity) to the amino acid sequence of wild-type mammalian (e.g., human or mouse) Atoh1 (e.g., SEQ ID NO: 8 or SEQ ID NO: 10) is operably linked to an SLC26A4 promoter described herein (e.g., a promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or a promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17), or is operably linked to a promoter (e.g., an SLC26A4 promoter, such as an SLC26A4 promoter or enhancer-promoter provided in Table 3, a minimal promoter, a core promoter, or a constitutive promoter) that is operably linked to one or more SLC26A4 enhancers described herein (e.g., one or more copies of an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 and/or SEQ ID NO: 3). In some embodiments, the polynucleotide encoding wild-type Atoh1, or a variant thereof is operably linked to a polynucleotide that contains, in 5′ to 3′ order, an enhancer that has the sequence of SEQ ID NO: 2, an enhancer that has the sequence of SEQ ID NO: 3, and a promoter that has the sequence of SEQ ID NO: 17. In some embodiments, the polynucleotide encoding wild-type Atoh1, or a variant thereof is operably linked to a polynucleotide having the sequence of SEQ ID NO: 18. In some embodiments, the polynucleotide sequence encoding an Atoh1 protein encodes an amino acid sequence that contains one or more conservative amino acid substitutions relative to SEQ ID NO: 4 (e.g., 1, 2, 3, 4, 5, 6, 7, 8, 9, 10, 11, 12, 13, 14, 15, 16, 17, 18, 19, 20 or more conservative amino acid substitutions), provided that the Atoh1 analog encoded retains the therapeutic function of wild-type Atoh1 (e.g., the ability to promote hair cell development). No more than 10% of the amino acids in the Atoh1 protein may be replaced with conservative amino acid substitutions. In some embodiments, the polynucleotide sequence that encodes Atoh1 is any polynucleotide sequence that, by redundancy of the genetic code, encodes SEQ ID NO: 8 or SEQ ID NO: 10. The polynucleotide sequence that encodes Atoh1 can be partially or fully codon-optimized for expression (e.g., in human SLC26A4-expressing inner ear cells, such as vestibular supporting cells). The Atoh1 protein may also be encoded by a polynucleotide having single nucleotide polymorphisms (SNPs) that have been found to be non-pathogenic in human subjects (e.g., SNPs that do not result in hearing loss). Human Atoh1 may be encoded by a polynucleotide having the sequence of SEQ ID NO: 9. Murine Atoh1 may be encoded by a polynucleotide having the sequence of SEQ ID NO: 11. The Atoh1 protein may be a human Atoh1 protein or may be a homolog of the human Atoh1 protein from another mammalian species (e.g., mouse, rat, cow, horse, goat, sheep, donkey, cat, dog, rabbit, guinea pig, or other mammal). Exemplary Atoh1 amino acid and polynucleotide sequences are listed in Table 5, below. A nucleic acid vector (e.g., an AAV vector) containing an SLC26A4 enhancer described herein operably linked to a promoter (e.g., an SLC26A4 promoter, such as an SLC26A4 promoter or enhancer-promoter provided in Table 3, a minimal promoter, a core promoter, or a constitutive promoter) that is operably inked to a polynucleotide encoding Atoh1, or a nucleic acid vector (e.g., an AAV vector) containing an SLC26A4 promoter described herein (e.g., SEQ ID NO: 1 or SEQ ID NO: 17) operably inked to a polynucleotide encoding Atoh1, can be administered to a subject to treat, reduce, or prevent vestibular dysfunction (e.g., imbalance or loss of balance, dizziness, or vertigo) associated with damage to or loss of vestibular hair cells (e.g., damage to or loss of vestibular hair cells related to head trauma, disease or infection, ototoxic drugs, or aging, e.g., age-related vestibular dysfunction, ototoxic-drug induced vestibular dysfunction, disease or infection-related vestibular dysfunction, or head trauma-related vestibular dysfunction).

TABLE 5
Atoh1 sequences

| SEQ | Description of | |
|---|---|---|
| ID NO: | sequence | Sequence |
| 8 | Human Atoh1 amino | MSRLLHAEEWAEVKELGDHHRQPQPHHLPQPPPPPQPPATLQARE |
| | acid sequence, | HPVYPPELSLLDSTDPRAWLAPTLOGICTARAAQYLLHSPELGASEA |
| | RefSeq accession | AAPRDEVDGRGELVRRSSGGASSSKSPGPVKVREQLCKLKGGVVV |
| | number | DELGCSRQRAPSSKQVNGVQKQRRLAANARERRRMHGLNHAFDQL |
| | NP_005163.1 | RNVIPSFNNDKKLSKYETLQMAQIYINALSELLQTPSGGEQPPPPPAS |
| | | CKSDHHHLRTAASYEGGAGNATAAGAQQASGGSQRPTPPGSCRTR |
| | | FSAPASAGGYSVQLDALHFSTFEDSALTAMMAQKNLSPSLPGSILQP |
| | | VQEENSKTSPRSHRSDGEFSPHSHYSDSDEAS |
| 9 | Human ATOH1 | ATGTCCCGCCTGCTGCATGCAGAAGAGTGGGCTGAAGTGAAGGA |
| | protein coding | GTTGGGAGACCACCATCGCCAGCCCCAGCCGCATCATCTCCCGC |
| | sequence, also | AACCGCCGCCGCCGCCGCAGCCACCTGCAACTTTGCAGGCGAGA |
| | documented under | GAGCATCCCGTCTACCCGCCTGAGCTGTCCCTCCTGGACAGCAC |
| | RefSeq accession | CGACCCACGCGCCTGGCTGGCTCCCACTTTGCAGGGCATCTGCA |
| | number | CGGCACGCGCCGCCCAGTATTTGCTACATTCCCCGGAGCTGGGT |
| | NM_005172.2 | GCCTCAGAGGCCGCTGCGCCCCGGGACGAGGTGGACGGCCGGG |
| | | GGGAGCTGGTAAGGAGGAGCAGCGGCGGTGCCAGCAGCAGCAA |
| | | GAGCCCCGGGCCGGTGAAAGTGCGGGAACAGCTGTGCAAGCTG |
| | | AAAGGGGGGGTGGTGGTAGACGAGCTGGGCTGCAGCCGCCAAC |
| | | GGGCCCCTTCCAGCAAACAGGTGAATGGGGTGCAGAAGCAGAGA |
| | | CGGCTAGCAGCCAACGCCAGGGAGCGGCGCAGGATGCATGGGC |
| | | TGAACCACGCCTTCGACCAGCTGCGCAATGTTATCCCGTCGTTCA |
| | | ACAACGACAAGAAGCTGTCCAAATATGAGACCCTGCAGATGGCCC |
| | | AAATCTACATCAACGCCTTGTCCGAGCTGCTACAAACGCCCAGCG |
| | | GAGGGGAACAGCCACCGCCGCCTCCAGCCTCCTGCAAAAGCGAC |
| | | CACCACCACCTTCGCACCGCGGCCTCCTATGAAGGGGGCGCGGG |
| | | CAACGCGACCGCAGCTGGGGCTCAGCAGGCTTCCGGAGGGAGC |
| | | CAGCGGCCGACCCCGCCCGGGAGTTGCCGGACTCGCTTCTCAGC |
| | | CCCAGCTTCTGCGGGAGGGTACTCGGTGCAGCTGGACGCTCTGC |
| | | ACTTCTCGACTTTCGAGGACAGCGCCCTGACAGCGATGATGGCG |
| | | CAAAAGAATTTGTCTCCTTCTCTCCCCGGGAGCATCTTGCAGCCA |
| | | GTGCAGGAGGAAAACAGCAAAACTTCGCCTCGGTCCCACAGAAG |
| | | CGACGGGGAATTTTCCCCCCATTCCCATTACAGTGACTCGGATGA |
| | | GGCAAGT |
| 10 | Murine Atoh1 amino | MSRLLHAEEWAEVKELGDHHRHPQPHHVPPLTPQPPATLQARDLPV |
| | acid sequence, | YPAELSLLDSTDPRAWLTPTLQGLCTARAAQYLLHSPELGASEAAAP |
| | UniProt P48985 | RDEADSQGELVRRSGCGGLSKSPGPVKVREQLCKLKGGVVVDELG |
| | | CSRQRAPSSKQVNGVQKQRRLAANARERRRMHGLNHAFDQLRNVI |
| | | PSFNNDKKLSKYETLQMAQIYINALSELLQTPNVGEQPPPPTASCKN |
| | | DHHHLRTASSYEGGAGASAVAGAQPAPGGGPRPTPPGPCRTRFSG |
| | | PASSGGYSVQLDALHFPAFEDRALTAMMAQKDLSPSLPGGILQPVQ |
| | | EDNSKTSPRSHRSDGEFSPHSHYSDSDEAS |
| 11 | Murine ATOH1 | ATGTCCCGCCTGCTGCATGCAGAAGAGTGGGCTGAGGTAAAAGA |
| | protein coding | GTTGGGGGACCACCATCGCCATCCCCAGCCGCACCACGTCCCGC |
| | sequence, also | CGCTGACGCCACAGCCACCTGCTACCCTGCAGGCGAGAGACCTT |
| | documented under | CCCGTCTACCCGGCAGAACTGTCCCTCCTGGATAGCACCGACCC |
| | RefSeq accession | ACGCGCCTGGCTGACTCCCACTTTGCAGGGCCTCTGCACGGCAC |
| | number | GCGCCGCCCAGTATCTGCTGCATTCTCCCGAGCTGGGTGCCTCC |
| | NM_007500.5 | GAGGCCGCGGCGCCCCGGGACGAGGCTGACAGCCAGGGTGAGC |
| | | TGGTAAGGAGAAGCGGCTGTGGCGGCCTCAGCAAGAGCCCCGG |
| | | GCCCGTCAAAGTACGGGAACAGCTGTGCAAGCTGAAGGGTGGGG |
| | | TTGTAGTGGACGAGCTTGGCTGCAGCCGCCAGCGAGCCCCTTCC |
| | | AGCAAACAGGTGAATGGGGTACAGAAGCAAAGGAGGCTGGCAGC |
| | | AAACGCAAGGGAACGGCGCAGGATGCACGGGCTGAACCACGCCT |
| | | TCGACCAGCTGCGCAACGTTATCCCGTCCTTCAACAACGACAAGA |
| | | AGCTGTCCAAATATGAGACCCTACAGATGGCCCAGATCTACATCA |
| | | ACGCTCTGTCGGAGTTGCTGCAGACTCCCAATGTCGGAGAGCAA |
| | | CCGCCGCCGCCCACAGCTTCCTGCAAAAATGACCACCATCACCTT |
| | | CGCACCGCCTCCTCCTATGAAGGAGGTGCGGGCGCCTCTGCGGT |
| | | AGCTGGGGCTCAGCCAGCCCCGGGAGGGGGCCCGAGACCTACC |
| | | CCGCCCGGGCCTTGCCGGACTCGCTTCTCAGGCCCAGCTTCCTC |
| | | TGGGGGTTACTCGGTGCAGCTGGACGCTTTGCACTTCCCAGCCTT |
| | | CGAGGACAGGGCCCTAACAGCGATGATGGCACAGAAGGACCTGT |
| | | CGCCTTCGCTGCCCGGGGGCATCCTGCAGCCTGTACAGGAGGAC |
| | | AACAGCAAAACATCTCCCAGATCCCACAGAAGTGACGGAGAGTTT |
| | | TCCCCCCACTCTCATTACAGTGACTCTGATGAGGCCAGT |

### Polynucleotides Encoding Proteins of Interest

One platform that can be used to achieve therapeutically effective intracellular concentrations of proteins of interest in mammalian cells is via the stable expression of the gene encoding the protein of interest (e.g., by integration into the nuclear or mitochondrial genome of a mammalian cell, or by episomal concatemer formation in the nucleus of a mammalian cell). The gene is a polynucleotide that encodes the primary amino acid sequence of the corresponding protein. In order to introduce exogenous genes into a mammalian cell, genes can be incorporated into a vector. Vectors can be introduced into a cell by a variety of methods, including transformation, transfection, transduction, direct uptake, projectile bombardment, and by encapsulation of the vector in a liposome. Examples of suitable methods of transfecting or transforming cells include calcium phosphate precipitation, electroporation, microinjection, infection, lipofection and direct uptake. Such methods are described in more detail, for example, in Green, et al., Molecular Cloning: A Laboratory Manual, Fourth Edition (Cold Spring Harbor University Press, New York 2014); and Ausubel, et al., Current Protocols in Molecular Biology (John Wiley & Sons, New York 2015), the disclosures of each of which are incorporated herein by reference.

Proteins of interest can also be introduced into a mammalian cell by targeting a vector containing a gene encoding a protein of interest to cell membrane phospholipids. For example, vectors can be targeted to the phospholipids on the extracellular surface of the cell membrane by linking the vector molecule to a VSV-G protein, a viral protein with affinity for all cell membrane phospholipids. Such a construct can be produced using methods well known to those of skill in the field.

Recognition and binding of the polynucleotide encoding a protein of interest by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase. Examples of mammalian promoters have been described in Smith, et al., Mol. Sys. Biol., 3:73, online publication, the disclosure of which is incorporated herein by reference. The promoter used in the methods and compositions described herein can be a SLC26A4 promoter (e.g., an SLC26A4 promoter or enhancer-promoter provided in Table 3), a constitutive promoter (e.g., a promoter active in vivo in all circumstances), a core promoter, or a minimal promoter. Constitutive promoters include the CAG promoter, a cytomegalovirus (CMV) promoter (e.g., the CMV immediate-early enhancer and promoter, a CMVmini promoter, a minCMV promoter, a CMV-TATA+INR promoter, or a min CMV-T6 promoter), the smCBA promoter (described in Haire et al., Invest. Opthalmol. Vis. Sci. 47:3745-3753, 2006), the CBA promoter, the CASI promoter, the dihydrofolate reductase (DHFR) promoter, the β-actin promoter, the phosphoglycerol kinase (PGK) promoter, a β-globin promoter (e.g., a minimal β-globin promoter), an HSV promoter (e.g., a minimal HSV ICP0 promoter or a truncated HSV ICP0 promoter), an SV40 promoter (e.g., an SV40 minimal promoter), and the EF1α promoter. Constitutive promoters may also be referred to as ubiquitous promoters for their ability to induce expression of a polynucleotide in a wide range of cell and tissue types. Minimal promoters include a CMV minimal promoter (e.g., a minCMV promoter), a minimal β-globin promoter, a minimal HSV promoter (e.g., a minimal HSV ICP0 promoter), and an SV40 minimal promoter. Alternatively, promoters derived from viral genomes can also be used for the stable expression of polynucleotides in mammalian (e.g., human) cells. Examples of functional viral promoters that can be used for the expression of polynucleotides in primate (e.g., human) cells include adenovirus late promoter, vaccinia virus 7.5K promoter, tk promoter of HSV, mouse mammary tumor virus (MMTV) promoter, LTR promoter of HIV, promoter of moloney virus, Epstein barr virus (EBV) promoter, and the Rous sarcoma virus (RSV) promoter.

Once a polynucleotide encoding a protein of interest has been incorporated into a mammalian cell, the transcription of this polynucleotide can be induced by methods known in the art. For example, expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms include tetracycline and doxycycline. These reagents are commercially available and can be administered to a mammalian cell in order to promote gene expression according to established protocols.

The nucleic acid vectors described herein may include a Woodchuck Posttranscriptional Regulatory Element (WPRE). The WPRE acts at the mRNA level, by promoting nuclear export of transcripts and/or by increasing the efficiency of polyadenylation of the nascent transcript, thus increasing the total amount of mRNA in the cell. The addition of the WPRE to a vector can result in a substantial improvement in the level of transgene expression from several different promoters, both in vitro and in vivo.

In some embodiments, the nucleic acid vectors described herein include a reporter sequence, which can be useful in verifying the expression of a gene operably linked to an SLC26A4 promoter and/or an SLC26A4 enhancer, for example, in cells and tissues (e.g., in SLC26A4-expressing cells, such as interdental cells, root cells, spiral prominence cells, and vestibular supporting cells). Reporter sequences that may be provided in a transgene include DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements that drive their expression, such as a promoter, the reporter sequences provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA), and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer.

### Methods for the Delivery of Exogenous Polynucleotides to Target Cells

Techniques that can be used to introduce a polynucleotide, such as a polynucleotide that is operably linked to an SLC26A4 promoter and/or an SLC26A4 enhancer described herein, into a target cell (e.g., a mammalian cell) are well known in the art. For instance, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous polynucleotides. Electroporation of mammalian cells is described in detail, e.g., in Chu et al., Nucleic Acids Research 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of target cells include the squeeze-poration methodology. This technique induces the rapid mechanical deformation of cells in order to stimulate the uptake of exogenous DNA through membranous pores that form in response to the applied stress. This technology is advantageous in that a vector is not required for delivery of polynucleotides into a cell, such as a human target cell. Squeeze-poration is described in detail, e.g., in Sharei et al., Journal of Visualized Experiments 81:e50980 (2013), the disclosure of which is incorporated herein by reference.

Lipofection represents another technique useful for transfection of target cells. This method involves the loading of polynucleotides into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous polynucleotides, for instance, by direct fusion of the liposome with the cell membrane or by endocytosis of the complex. Lipofection is described in detail, for instance, in U.S. Pat. No. 7,442,386, the disclosure of which is incorporated herein by reference. Similar techniques that exploit ionic interactions with the cell membrane to provoke the uptake of foreign polynucleotides include contacting a cell with a cationic polymer-polynucleotide complex. Exemplary cationic molecules that associate with polynucleotides so as to impart a positive charge favorable for interaction with the cell membrane include activated dendrimers (described, e.g., in Dennig, Topics in Current Chemistry 228:227 (2003), the disclosure of which is incorporated herein by reference) polyethylenimine, and diethylaminoethyl (DEAE)-dextran, the use of which as a transfection agent is described in detail, for instance, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of polynucleotides. This technology is described in detail, for instance, in US 2010/0227406, the disclosure of which is incorporated herein by reference.

Another useful tool for inducing the uptake of exogenous polynucleotides by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107: 1870 (2010), the disclosure of which is incorporated herein by reference.

Magnetofection can also be used to deliver polynucleotides to target cells. The magnetofection principle is to associate polynucleotides with cationic magnetic nanoparticles. The magnetic nanoparticles are made of iron oxide, which is fully biodegradable, and coated with specific cationic proprietary molecules varying upon the applications. Their association with the gene vectors (DNA, siRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Therapy 9:102 (2002), the disclosure of which is incorporated herein by reference.

Another useful tool for inducing the uptake of exogenous polynucleotides by target cells is sonoporation, a technique that involves the use of sound (typically ultrasonic frequencies) for modifying the permeability of the cell plasma membrane to permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a target cell according to the methods described herein. For instance, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Vectors for Delivery of Exogenous Polynucleotides to Target Cells

In addition to achieving high rates of transcription and translation, stable expression of an exogenous polynucleotide in a mammalian cell can be achieved by integration of the polynucleotide into the nuclear genome of the mammalian cell. A variety of vectors for the delivery and integration of polynucleotides encoding expression products into the nuclear DNA of a mammalian cell have been developed. Examples of expression vectors are described in, e.g., Gellissen, Production of Recombinant Proteins: Novel Microbial and Eukaryotic Expression Systems (John Wiley & Sons, Marblehead, MA, 2006). Expression vectors for use in the compositions and methods described herein contain at least one SLC26A4 enhancer (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or SEQ ID NO: 3) and/or an SLC26A4 promoter (e.g., a promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17) operably linked polynucleotide encoding an expression product (e.g., a polynucleotide that encodes a protein of interest or that can be transcribed to produce an RNA molecule, such as an inhibitory RNA), as well as, e.g., additional sequence elements used for the expression of these agents and/or the integration of these polynucleotide sequences into the genome of a mammalian cell. Vectors that can contain one or more SLC26A4 enhancers and/or an SLC26A4 promoter operably linked to polynucleotide encoding an expression product (e.g., a transgene encoding a protein of interest) include plasmids (e.g., circular DNA molecules that can autonomously replicate inside a cell), cosmids (e.g., pWE or sCos vectors), artificial chromosomes (e.g., a human artificial chromosome (HAC), a yeast artificial chromosome (YAC), a bacterial artificial chromosome (BAC), or a P1-derived artificial chromosome (PAC)), and viral vectors. Certain vectors that can be used for the expression of an expression product (e.g., a protein of interest) include plasmids that contain regulatory sequences that direct gene transcription. Other useful vectors for expression of an expression product (e.g., a protein of interest) contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

### Viral Vectors for Polynucleotide Delivery

Viral genomes provide a rich source of vectors that can be used for the efficient delivery of a gene of interest into the genome of a target cell (e.g., a mammalian cell, such as a human cell). Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, 1996)). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, U.S. Pat. No. 5,801,030, the disclosure of which is incorporated herein by reference as it pertains to viral vectors for use in gene therapy.

### AAV Vectors for Polynucleotide Delivery

In some embodiments, polynucleotides of the compositions and methods described herein are incorporated into rAAV vectors and/or virions in order to facilitate their introduction into a cell. rAAV vectors useful in the compositions and methods described herein are recombinant polynucleotide constructs that include (1) a promoter (e.g., an SLC26A4 promoter, e.g., a promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or a promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17), (2) a sequence to be expressed (e.g., a polynucleotide encoding a protein, such as pendrin or Atoh1, or a polynucleotide that can be transcribed to produce an RNA molecule, such as an inhibitory RNA), and (3) viral sequences that facilitate integration and expression of the sequence to be expressed. The viral sequences may include those sequences of AAV that are required in cis for replication and packaging (e.g., functional ITRs) of the DNA into a virion. In some embodiments, the rAAV vectors further include at least one SLC26A4 enhancer described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or SEQ ID NO: 3). In some embodiments, rAAV vectors useful in the compositions and methods described herein contain, in 5′ to 3′ order, an enhancer that has the sequence of SEQ ID NO: 2, an enhancer that has the sequence of SEQ ID NO: 3, and a promoter that has the sequence of SEQ ID NO: 17. In some embodiments the rAAV vector contains the sequence of SEQ ID NO: 18. In typical applications, the sequence to be expressed encodes a wild-type form of a protein expressed in SLC26A4-expressing inner ear cells that is mutated in subjects with forms of hereditary hearing loss, such as a wild-type form of pendrin, or a protein or RNA molecule that can promote the differentiation of vestibular supporting cells into vestibula hair cells, such as Atoh1. Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. For use in the methods and compositions described herein, the ITRs can be AAV2 ITRs. Methods for using rAAV vectors are described, for example, in Tal et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The polynucleotides and vectors described herein (e.g., an SLC26A4 enhancer and/or an SLC26A4 promoter operably linked to a polynucleotide encoding an expression product) can be incorporated into a rAAV virion in order to facilitate introduction of the polynucleotide or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for instance, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, 9, 10, 11, rh10, rh39, rh43, rh74, Anc80, Anc80L65, DJ, DJ/8, DJ/9, 7m8, PHP.B, PHP.eb, and PHP.S. For targeting SLC26A4-expressing cells, AAV1, AAV2, AAV2quad(Y-F), AAV6, AAV8, AAV9, Anc80, Anc80L65, AAV-DJ, AAV-DJ/9, 7m8, and PHP.B may be particularly useful. Serotypes evolved for transduction of the retina may also be used in the methods and compositions described herein. Construction and use of AAV vectors and AAV proteins of different serotypes are described, for instance, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV2quad(Y-F), AAV3, AAV4, AAV5, AAV6, AAV7, AAV8, etc.). Techniques involving the construction and use of pseudotyped rAAV virions are known in the art and are described, for instance, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

### Pharmaceutical Compositions

The SLC26A4 enhancers (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or SEQ ID NO: 3) and/or SLC26A4 promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17) described herein may be operably linked to a polynucleotide encoding an expression product (e.g., a transgene encoding a protein of interest or an RNA molecule, such as an inhibitory RNA) and incorporated into a vehicle for administration to a patient, such as a human patient suffering from sensorineural hearing loss (e.g., pendrin-related hearing loss) or vestibular dysfunction (e.g., vestibular dysfunction associated with damage to or loss of vestibular hair cells or pendrin-related vestibular dysfunction). Pharmaceutical compositions containing vectors, such as viral vectors, that contain an SLC26A4 enhancer and/or an SLC26A4 promoter described herein operably linked to a polynucleotide encoding an expression product can be prepared using methods known in the art. For example, such compositions can be prepared using, e.g., physiologically acceptable carriers, excipients, or stabilizers (Remington: The Science and Practice of Pharmacology 22nd edition, Allen, L. Ed. (2013); incorporated herein by reference), and in a desired form, e.g., in the form of lyophilized formulations or aqueous solutions.

Mixtures of nucleic acid vectors (e.g., viral vectors) containing an SLC26A4 enhancer (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or SEQ ID NO: 3) and/or a SLC26A4 promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17) described herein operably linked to a polynucleotide encoding an expression product may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. For local administration to the inner ear, the composition may be formulated to contain a synthetic perilymph solution. An exemplary synthetic perilymph solution includes 20-200 mM NaCl, 1-5 mM KCl, 0.1-10 mM CaCl₂), 1-10 mM glucose, and 2-50 mM HEPEs, with a pH between about 6 and 9 and an osmolality of about 300 mOsm/kg. The person responsible for administration will, in any event, determine the appropriate dose for the individual subject. Moreover, for human administration, preparations may meet sterility, pyrogenicity, general safety, and purity standards as required by FDA Office of Biologics standards.

### Methods of Treatment

The compositions described herein may be administered to a subject having or at risk of developing sensorineural hearing loss, vestibular dysfunction, or Meniere's disease by a variety of routes, such as local administration to the middle or inner ear (e.g., administration into the perilymph or endolymph, such as to or through the oval window, round window, or semicircular canal (e.g., the horizontal canal), or by transtympanic or intratympanic injection, e.g., administration to an SLC26A4-expressing inner ear cell), intravenous, parenteral, intradermal, transdermal, intramuscular, intranasal, subcutaneous, percutaneous, intratracheal, intraperitoneal, intraarterial, intravascular, inhalation, perfusion, lavage, and oral administration. The most suitable route for administration in any given case will depend on the particular composition administered, the patient, pharmaceutical formulation methods, administration methods (e.g., administration time and administration route), the patient's age, body weight, sex, severity of the disease being treated, the patient's diet, and the patient's excretion rate. Compositions may be administered once, or more than once (e.g., once annually, twice annually, three times annually, bi-monthly, monthly, or bi-weekly).

Subjects that may be treated as described herein are subjects having or at risk of developing sensorineural hearing loss. In some embodiments, the compositions described herein are used to treat pendrin-related hearing loss (e.g., DFNB4 or Pendred syndrome). DFNB4 and Pendred syndrome can be treated by administration of a nucleic acid vector containing an SLC26A4 enhancer (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or SEQ ID NO: 3) and/or an SLC26A4 promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17) described herein operably linked to a polynucleotide encoding pendrin (e.g., a polynucleotide encoding SEQ ID NO: 4 or SEQ ID NO: 5). In some embodiments, the nucleic acid vector contains, in 5′ to 3′ order, an enhancer that has the sequence of SEQ ID NO:2, an enhancer that has the sequence of SEQ ID NO:3, and a promoter that has the sequence of SEQ ID NO: 17 operably linked to a polynucleotide encoding pendrin. In some embodiments the nucleic acid vector contains a polynucleotide having the sequence of SEQ ID NO: 18 operably linked to a polynucleotide encoding pendrin. The subject may have or be identified as having a mutation in SLC26A4 and may have severe, moderate, or mild hearing loss when treatment is initiated or may be treated prior to symptom onset (e.g., preventative treatment). In some embodiments, the compositions are administered as a preventative treatment to a subject at risk of developing hearing loss, e.g., a subject carrying a mutation in SLC26A4 that is associated with hearing loss who does not yet exhibit hearing impairment.

In some embodiments, the compositions described herein are used to treat a subject having Meniere's disease. Both subjects with mutations in SLC26A4 and subjects with Meniere's disease have endolymphatic hydrops, accordingly, compositions that can be used to treat subjects with mutations in SLC26A4 (e.g., a nucleic acid vector containing an SLC26A4 enhancer (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or SEQ ID NO: 3) and/or an SLC26A4 promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17) described herein operably linked to a polynucleotide encoding pendrin (e.g., a polynucleotide encoding SEQ ID NO: 4 or SEQ ID NO: 5)) may also be effective in reducing or ameliorating endolymphatic hydrops in subjects with Meniere's disease. Such treatment may be used to treat hearing loss, tinnitus, or vestibular dysfunction (e.g., vertigo) in a subject with Meniere's disease and may reduce the feeling of fullness or congestion in the ear.

In some embodiments, the compositions described herein are used to treat a subject having or at risk of developing vestibular dysfunction (e.g., vertigo, dizziness, imbalance, oscillopsia, a balance disorder, or bilateral vestibulopathy). In some embodiments, the vestibular dysfunction is pendrin-related vestibular dysfunction associated with DFNB4 or Pendred syndrome (e.g., imbalance or loss of balance associated with DFNB4 or Pendred syndrome). Pendrin-related vestibular dysfunction can be treated by administration of a nucleic acid vector containing an SLC26A4 enhancer (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or SEQ ID NO: 3) and/or an SLC26A4 promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17) described herein operably linked to a polynucleotide encoding pendrin (e.g., a polynucleotide encoding SEQ ID NO: 4 or SEQ ID NO: 5). In some embodiments, the subject may have or may have been identified as having a mutation in SLC26A4 and may have severe, moderate, or mild vestibular dysfunction when treatment is initiated or may be treated prior to symptom onset (e.g., preventative treatment).

Vestibular dysfunction may also result from damage to or loss of vestibular hair cells. Accordingly, the compositions and methods described herein can be used to treat a subject having or at risk of developing damage to or loss of vestibular hair cells (e.g., damage to or loss of vestibular hair cells related to disease or infection, head trauma, ototoxic drugs (e.g., vestibulotoxic drugs), or aging), subjects having or at risk of developing vestibular dysfunction (e.g., dizziness, vertigo, imbalance, bilateral vestibulopathy, oscillopsia, or a balance disorder), subjects carrying a genetic mutation associated with vestibular dysfunction, or subjects with a family history of hereditary vestibular dysfunction. In some embodiments, the disease associated with damage to or loss of hair cells (e.g., vestibular hair cells) is an autoimmune disease or condition in which an autoimmune response contributes to hair cell damage or death. Autoimmune diseases linked to vestibular dysfunction include autoimmune inner ear disease (AIED), polyarteritis nodosa (PAN), Cogan's syndrome, relapsing polychondritis, systemic lupus erythematosus (SLE), Wegener's granulomatosis, Sjögren's syndrome, and Behçet's disease. Some infectious conditions, such as Lyme disease and syphilis can also cause vestibular dysfunction (e.g., by triggering autoantibody production). Viral infections, such as rubella, cytomegalovirus (CMV), lymphocytic choriomeningitis virus (LCMV), HSV types 1 &2, West Nile virus (WNV), human immunodeficiency virus (HIV) varicella zoster virus (VZV), measles, and mumps, can also cause vestibular dysfunction. In some embodiments, the subject has vestibular dysfunction that is associated with or results from loss of hair cells (e.g., vestibular hair cells). In some embodiments, compositions and methods described herein can be used to treat a subject having or at risk of developing oscillopsia. In some embodiments, compositions and methods described herein can be used to treat a subject having or at risk of developing bilateral vestibulopathy. In some embodiments, the compositions and methods described herein can be used to treat a subject having or at risk of developing a balance disorder (e.g., imbalance). The compositions and methods described herein may also be administered as a preventative treatment to subjects at risk of developing vestibular dysfunction, e.g., subjects who have a family history of vestibular dysfunction (e.g., inherited vestibular dysfunction), subjects carrying a genetic mutation associated with vestibular dysfunction who do not yet exhibit symptoms of vestibular dysfunction, or subjects exposed to risk factors for acquired vestibular dysfunction (e.g., disease or infection, head trauma, ototoxic drugs, or aging). The compositions and methods described herein can also be used to treat a subject with idiopathic vestibular dysfunction.

The compositions and methods described herein can be used to induce or increase vestibular hair cell regeneration in a subject. Subjects that may benefit from compositions that promote or induce vestibular hair cell regeneration, include subjects having or at risk of developing vestibular dysfunction as a result of loss of hair cells (e.g., loss of vestibular hair cells related to trauma (e.g., head trauma), disease or infection, ototoxic drugs, or aging), and subjects with abnormal vestibular hair cells (e.g., vestibular hair cells that do not function properly compared to normal vestibular hair cells), damaged vestibular hair cells (e.g., vestibular hair cell damage related to trauma (e.g., head trauma), disease or infection, ototoxic drugs, or aging), or reduced vestibular hair cell numbers due to a genetic mutation or a congenital abnormality. The compositions and methods described herein can also be used to promote or increase vestibular hair cell maturation, which can lead to improved vestibular function. In some embodiments, the compositions and methods described herein promote or increase the maturation of regenerated vestibular hair cells.

The compositions and methods described herein can also be used to prevent or reduce vestibular dysfunction caused by ototoxic drug-induced vestibular hair cell damage or death (e.g., vestibulotoxic drug-induced vestibular hair loss) in subjects who have been treated with ototoxic drugs, or who are currently undergoing or soon to begin treatment with ototoxic drugs. Ototoxic drugs are toxic to the cells of the inner ear, and can cause vestibular dysfunction (e.g., vertigo, dizziness, imbalance, bilateral vestibulopathy, or oscillopsia). Drugs that have been found to be ototoxic include aminoglycoside antibiotics (e.g., gentamycin, neomycin, streptomycin, tobramycin, kanamycin, vancomycin, amikacin, dibekacin, and netilmicin), viomycin, antineoplastic drugs (e.g., platinum-containing chemotherapeutic agents, such as cisplatin, carboplatin, and oxaliplatin, or other chemotherapeutic agents, such as nitrogen mustards and vincristine), loop diuretics (e.g., ethacrynic acid and furosemide), salicylates (e.g., aspirin, particularly at high doses), and quinine. Certain of these drugs, such as nitrogen mustards, vincristine, gentamicin, streptomycin, and tobramycin, have been specifically identified as vestibulotoxic drugs. In some embodiments, the methods and compositions described herein can be used to treat bilateral vestibulopathy or oscillopsia due to aminoglycoside ototoxicity (e.g., the methods and compositions described herein can be used to promote or increase vestibular hair cell regeneration in a subject with aminoglycoside-induced bilateral vestibulopathy or oscillopsia).

Vestibular dysfunction associated with damage to or loss of vestibular hair cells (e.g., damage to or loss of vestibular hair cells related to disease or infection, head trauma, ototoxic drugs (e.g., vestibulotoxic drugs), or aging) can be treated by administration of a nucleic acid vector containing an SLC26A4 enhancer (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or SEQ ID NO: 3) and/or an SLC26A4 promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17) described herein operably linked to a polynucleotide encoding Atoh1 (e.g., a polynucleotide encoding SEQ ID NO: 8 or SEQ ID NO: 10) or pendrin (e.g., a polynucleotide encoding SEQ ID NO: 4 or SEQ ID NO: 5). In some embodiments, the nucleic acid vector contains, in 5′ to 3′ order, an enhancer that has the sequence of SEQ ID NO: 2, an enhancer that has the sequence of SEQ ID NO: 3, and a promoter that has the sequence of SEQ ID NO: 17 operably linked to a polynucleotide encoding Atoh1 or pendrin. In some embodiments the nucleic acid vector contains a polynucleotide having the sequence of SEQ ID NO: 18 operably linked to a polynucleotide encoding Atoh1 or pendrin. Such a nucleic acid vector can also be used to induce or increase vestibular hair cell regeneration or vestibular hair cell maturation in a subject in need thereof.

The methods described herein may include a step of screening a subject for one or more mutations in genes known to be associated with hearing loss or vestibular dysfunction (e.g., SLC26A4) prior to treatment with or administration of the compositions described herein. A subject can be screened for a genetic mutation using standard methods known to those of skill in the art (e.g., genetic testing). The methods described herein may also include a step of assessing hearing in a subject prior to treatment with or administration of the compositions described herein. Hearing can be assessed using standard tests, such as audiometry, auditory brainstem response (ABR), electrocochleography (ECOG), and otoacoustic emissions. These tests can also be used to assess hearing in a subject after treatment with or administration of the compositions described herein. In some embodiments, the methods described herein include a step of assessing vestibular function in a subject prior to treatment with or administration of the compositions described herein. Vestibular function may be assessed using standard tests, such as eye movement testing (e.g., electronystagmogram (ENG) or videonystagmogram (VNG)), tests of the vestibulo-ocular reflex (VOR) (e.g., the head impulse test (Halmagyi-Curthoys test), which can be performed at the bedside or using a video-head impulse test (VHIT), or the caloric reflex test), posturography, rotary-chair testing, ECOG, vestibular evoked myogenic potentials (VEMP), and specialized clinical balance tests, such as those described in Mancini and Horak, Eur J Phys Rehabil Med, 46:239 (2010). These tests can also be used to assess vestibular function in a subject after treatment with or administration of the compositions described herein.

The polynucleotide encoding an expression product operably linked to an SLC26A4 promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17) and/or an SLC26A4 enhancer (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or SEQ ID NO: 3) for treatment of a subject as described herein can be a polynucleotide encoding pendrin (e.g., a polynucleotide encoding the amino acid sequence of SEQ ID NO: 4 or SEQ ID NO: 5), a polynucleotide encoding Atoh1 (e.g., a polynucleotide encoding the amino acid sequence of SEQ ID NO: 8 or SEQ ID NO: 10), a polynucleotide encoding the wild-type version of a protein expressed in SLC26A4-expressing inner ear cells that is mutated in a subject with sensorineural hearing loss or vestibular dysfunction, a polynucleotide that encodes another protein of interest (e.g., a reporter protein, such as a fluorescent protein, lacZ, or luciferase), or a polynucleotide that can be transcribed to produce an RNA molecule, such as an shRNA, an ASO, a component of a gene editing system (e.g., a nuclease, such as a CRISPR Associated Protein 9 (Cas9), Transcription Activator-Like Effector Nuclease (TALEN), or Zinc Finger Nuclease (ZFN), or a guide RNA (gRNA)), or a microRNA. In some embodiments, the expression product is operably linked to a polynucleotide sequence comprising, in 5′ to 3′ order, an enhancer that has the sequence of SEQ ID NO: 2, an enhancer that has the sequence of SEQ ID NO: 3, and a promoter that has the sequence of SEQ ID NO:17. In some embodiments the expression product is operably linked to a polynucleotide having the sequence of SEQ ID NO: 18. The polynucleotide may be selected based on the cause of the subject's hearing loss or vestibular dysfunction (e.g., if the subject's hearing loss is associated with a mutation in SLC26A4, the polynucleotide can encode wild-type pendrin, or if the subject's vestibular dysfunction is age-related or ototoxic drug-induced vestibular dysfunction associated with loss of hair cells, the polynucleotide can encode Atoh1), the severity of the subject's hearing loss, the health of the subject's inner ear cells, the subject's age, the subject's family history of hearing loss, or other factors.

Treatment may include administration of a composition containing a nucleic acid vector (e.g., an AAV vector) containing an SLC26A4 enhancer and/or an SLC26A4 promoter described herein in various unit doses. Each unit dose will ordinarily contain a predetermined quantity of the therapeutic composition. The quantity to be administered, and the particular route of administration and formulation, are within the skill of those in the clinical arts. A unit dose need not be administered as a single injection but may comprise continuous infusion over a set period of time. Dosing may be performed using a syringe pump to control infusion rate in order to minimize damage to the inner ear (e.g., the cochlea and/or vestibular system). In cases in which the nucleic acid vectors are AAV vectors (e.g., AAV1, AAV2, AAV2quad(Y-F), AAV3, AAV4, AAV5, AAV6, AAV7, AAV8, AAV9, AAV10, rh10, rh39, rh43, rh74, Anc80, Anc80L65, DJ, DJ/8, DJ/9, 7m8, PHP.B, PHP.eb, or PHP.S vectors), the viral vectors may be administered to the patient at a dose of, for example, from about 1×10⁹vector genomes (VG)/mL to about 1×10¹⁶ VG/mL (e.g., 1×10⁹ VG/mL, 2×10⁹ VG/mL, 3×10⁹ VG/mL, 4×10⁹ VG/mL, 5×10⁹ VG/mL, 6×10⁹ VG/mL, 7×10⁹ VG/mL, 8×10⁹ VG/mL, 9×10⁹ VG/mL, 1×10¹⁰ VG/mL, 2×10¹⁰ VG/mL, 3×10¹⁰ VG/mL, 4×10¹⁰ VG/mL, 5×10¹⁰ VG/mL, 6×10¹⁰ VG/mL, 7×10¹⁰ VG/mL, 8×10¹⁰ VG/mL, 9×10¹⁰ VG/mL, 1×10¹¹ VG/mL, 2×10¹¹ VG/mL, 3×10¹¹ VG/mL, 4×10¹¹ VG/mL, 5×10¹¹ VG/mL, 6×10¹¹ VG/mL, 7×10¹¹ VG/mL, 8×10¹¹ VG/mL, 9×10¹¹ VG/mL, 1×10¹² VG/mL, 2×10¹² VG/mL, 3×10¹² VG/mL, 4×10¹² VG/mL, 5×10¹² VG/mL, 6×10¹² VG/mL, 7×10¹² VG/mL, 8×10¹² VG/mL, 9×10¹² VG/mL, 1×10¹³ VG/mL, 2×10¹³ VG/mL, 3×10¹³ VG/mL, 4×10¹³ VG/mL, 5×10¹³ VG/mL, 6×10¹³ VG/mL, 7×10¹³ VG/mL, 8×10¹³ VG/mL, 9×10¹³ VG/mL, 1×10¹⁴ VG/mL, 2×10¹⁴ VG/mL, 3×10¹⁴ VG/mL, 4×10¹⁴ VG/mL, 5×10¹⁴ VG/mL, 6×10¹⁴ VG/mL, 7×10¹⁴ VG/mL, 8×10¹⁴ VG/mL, 9×10¹⁴ VG/mL, 1×10¹⁵ VG/mL, 2×10¹⁵ VG/mL, 3×10¹⁵ VG/mL, 4×10¹⁵ VG/mL, 5×10¹⁵ VG/mL, 6×10¹⁵ VG/mL, 7×10¹⁵ VG/mL, 8×10¹⁵ VG/mL, 9×10¹⁵ VG/mL, or 1×10¹⁶ VG/mL) in a volume of 1 μL to 200 μL (e.g., 1, 2, 3, 5, 6, 7, 8, 9, 10, 15, 20, 25, 30, 35, 40, 45, 50, 55, 60, 65, 70, 75, 80, 85, 90, 95, 100, 110,120, 130,140,150,160,170, 180, 190, or 200 μL). The AAV vectors may be administered to the subject at a dose of about 1×10⁷ VG/ear to about 2×10¹⁵ VG/ear (e.g., 1×10⁷ VG/ear, 2×10⁷ VG/ear, 3×10⁷ VG/ear, 4×10⁷ VG/ear, 5×10⁷ VG/ear, 6×10⁷ VG/ear, 7×10⁷ VG/ear, 8×10⁷ VG/ear, 9×10⁷ VG/ear, 1×10⁸ VG/ear, 2×10⁸ VG/ear, 3×10⁸ VG/ear, 4×10⁸ VG/ear, 5×10⁸ VG/ear, 6×10⁸ VG/ear, 7×10⁸ VG/ear, 8×10⁸ VG/ear, 9×10⁸ VG/ear, 1×10⁹ VG/ear, 2×10⁹ VG/ear, 3×10⁹ VG/ear, 4×10⁹ VG/ear, 5×10⁹ VG/ear, 6×10⁹ VG/ear, 7×10⁹ VG/ear, 8×10⁹ VG/ear, 9×10⁹ VG/ear, 1×10¹⁰VG/ear, 2×10¹⁰ VG/ear, 3×10¹⁰ VG/ear, 4×10¹⁰ VG/ear, 5×10¹⁰ VG/ear, 6×10¹⁰ VG/ear, 7×10¹⁰ VG/ear, 8×10¹⁰ VG/ear, 9×10¹⁰VG/ear, 1×10¹¹ VG/ear, 2×10¹¹ VG/ear, 3×10¹¹ VG/ear, 4×10¹¹ VG/ear, 5×10¹¹ VG/ear, 6×10¹¹ VG/ear, 7×10¹¹ VG/ear, 8×10¹¹ VG/ear, 9×10¹¹ VG/ear, 1×10¹² VG/ear, 2×10¹² VG/ear, 3×10¹² VG/ear, 4×10¹² VG/ear, 5×10¹² VG/ear, 6×10¹² VG/ear, 7×10¹² VG/ear, 8×10¹² VG/ear, 9×10¹² VG/ear, 1×10¹³ VG/ear, 2×10¹³ VG/ear, 3×10¹³ VG/ear, 4×10¹³ VG/ear, 5×10¹³ VG/ear, 6×10¹³ VG/ear, 7×10¹³ VG/ear, 8×10¹³ VG/ear, 9×10¹³ VG/ear, 1×10¹⁴ VG/ear, 2×10¹⁴ VG/ear, 3×10¹⁴ VG/ear, 4×10¹⁴ VG/ear, 5×10¹⁴ VG/ear, 6×10¹⁴ VG/ear, 7×10¹⁴ VG/ear, 8×10¹⁴ VG/ear, 9×10¹⁴ VG/ear, 1×10¹⁵ VG/ear, or 2×10¹⁵ VG/ear).

The compositions described herein are administered in an amount sufficient to improve or restore (e.g., rescue) hearing, inhibit or slow the progression of hearing loss (e.g., sensorineural hearing loss), reduce tinnitus (e.g., in a subject with Meniere's disease), reduce vestibular dysfunction, improve vestibular function (e.g., improve balance or reduce dizziness or vertigo), treat bilateral vestibulopathy, treat oscillopsia, inhibit or slow the progression of vestibular dysfunction, reduce the feeling of fullness in the ear (e.g., in a subject with Meniere's disease), increase or promote vestibular hair cell regeneration, increase or induce hair cell maturation (e.g., the maturation of regenerated vestibular hair cells), or increase or induce expression of an expression product in SLC26A4-expressing cells (e.g., interdental cells, spiral prominence cells, root cells, or vestibular supporting cells). Hearing may be evaluated using standard hearing tests (e.g., audiometry, ABR, electrocochleography (ECOG), and otoacoustic emissions) and may be improved by 5% or more (e.g., 5%, 10%, 15%, 20%, 30%, 40%, 50%, 60%, 70%, 80%, 90%, 100%, 125%, 150%, 200% or more) compared to hearing measurements obtained prior to treatment. In some embodiments, the compositions are administered in an amount sufficient to improve the subject's ability to understand speech. The compositions described herein may also be administered in an amount sufficient to delay or prevent the development of sensorineural hearing loss or deafness (e.g., in subjects who carry an SLC26A4 mutation but do not exhibit hearing impairment at the time of treatment, or in subjects exhibiting mild to moderate hearing loss at the time of treatment). Vestibular function may be evaluated using standard tests for balance and vertigo (e.g., eye movement testing (e.g., ENG or VNG), VOR testing (e.g., head impulse testing (Halmagyi-Curthoys testing, e.g., VHIT), or caloric reflex testing), posturography, rotary-chair testing, ECOG, VEMP, and specialized clinical balance tests) and may be improved by 5% or more (e.g., 5%, 10%, 15%, 20%, 30%, 40%, 50%, 60%, 70%, 80%, 90%, 100%, 125%, 150%, 200% or more) compared to measurements obtained prior to treatment. The compositions described herein may also be administered in an amount sufficient to slow or prevent the development or progression of vestibular dysfunction (e.g., in subjects who carry a mutation in SLC26A4 associated with vestibular dysfunction, or who have been exposed to risk factors associated with vestibular dysfunction (e.g., ototoxic drugs, head trauma, or disease or infection) but who do not exhibit vestibular dysfunction (e.g., vertigo, dizziness, or imbalance), or in subjects exhibiting mild to moderate vestibular dysfunction). Expression of a protein encoded by a transgene operably linked to a SLC26A4 promoter and/or enhancer in a nucleic acid vector administered to the subject or to a cell may be evaluated using immunohistochemistry, Western blot analysis, quantitative real-time PCR, or other methods known in the art for detection protein or mRNA, and may be increased by 5% or more (e.g., 5%, 10%, 15%, 20%, 30%, 40%, 50%, 60%, 70%, 80%, 90%, 100%, 125%, 150%, 200% or more) compared to expression prior to administration of the compositions described herein. Vestibular hair cell regeneration may be evaluated indirectly based on tests of vestibular function, and may be increased by 5% or more (e.g., 5%, 10%, 15%, 20%, 30%, 40%, 50%, 60%, 70%, 80%, 90%, 100%, 125%, 150%, 200% or more) compared to vestibular hair cell regeneration prior to administration of a composition described herein or compared to an untreated subject. The compositions and methods described herein may also reduce the toxicity associated with administration of a nucleic acid vector compared to the toxicity observed after the administration of a nucleic acid vector that does not contain an SLC26A4 promoter and/or enhancer described herein (e.g., administration of a nucleic acid vector in which the same transgene is expressed using a ubiquitous promoter and/or without an SLC26A4 enhancer described herein). These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the compositions described herein. The patient may be evaluated 1 month, 2 months, 3 months, 4 months, 5 months, 6 months, or more following administration of the composition depending on the dose and route of administration used for treatment. Depending on the outcome of the evaluation, the patient may receive additional treatments.

### Kits

The compositions described herein can be provided in a kit for use in treating sensorineural hearing loss or vestibular dysfunction. Compositions may include one or more SLC26A4 enhancers (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or SEQ ID NO: 3) and/or an SLC26A4 promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17) described herein, a nucleic acid vector containing such a polynucleotide, or a nucleic acid vector containing an SLC26A4 enhancer and/or promoter described herein that is operably linked to a polynucleotide encoding an expression product (e.g., a transgene encoding a protein of interest, such as a protein that can be expressed in SLC26A4-expressing inner ear cells to treat hearing loss (e.g., pendrin) or vestibular dysfunction (e.g., pendrin or Atoh1), or a transgene encoding an RNA molecule, such as an inhibitory RNA molecule). The nucleic acid vector may be packaged in an AAV virus capsid (e.g., AAV1, AAV2, AAV2quad(Y-F), AAV6, AAV8, AAV9, Anc80, Anc80L65, AAV-DJ, AAV-DJ/9, 7m8, or PHP.B). The kit can further include a package insert that instructs a user of the kit, such as a physician, to perform the methods described herein. The kit may optionally include a syringe or other device for administering the composition.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Demonstration of AAV1-CMV.H2B.EGFP Tropism Across Mouse Inner Ear Tissue

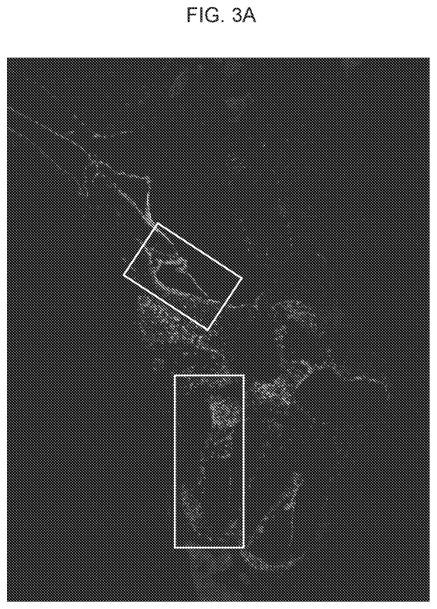

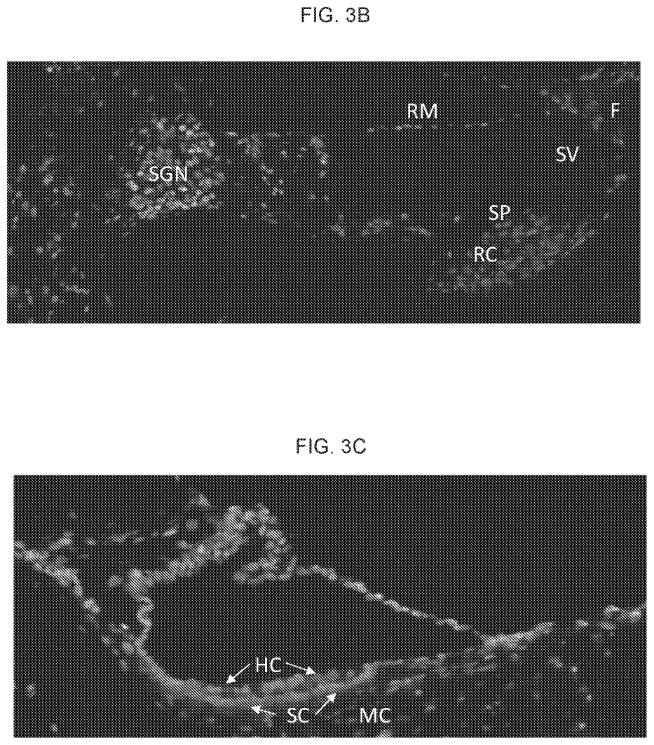

To test the hypothesis that AAV1 transduces a wide array of cell types across the inner ear, including cell types that do not express Pendrin, an AAV1 virus was delivered to adult C57BL/6 mice via the posterior semicircular canal (intra-labyrinth (IL)) at a dose of 7.19×10¹⁰ vg/ear. This AAV1 virus was packaged with a plasmid containing an expression cassette encoding the cytomegalovirus (CMV) promoter driving expression of a nuclear-targeted enhanced green fluorescent protein (EGFP fused to the H2B fragment of the histone 2b gene) at a titer of 3.59×10¹³ vg/mL. Two weeks after administration of the virus, whole ears were fixed, decalcified, and paraffin-embedded, and sections were imaged for EGFP using fluorescence microscopy. EGFP fluorescence was detected in the nuclei of many cell types across the inner ear including, but not limited to, spiral ganglion neurons, Reissner's membrane, fibrocytes of the lateral wall, spiral prominence cells, and root cells of the cochlea (FIGS. 3A and 3B). In the vestibule, EGFP fluorescence was detected in the nuclei of hair cells, supporting cells, mesenchymal cells, and roof cells of the otolith organs (FIGS. 3A and 3C).

### Example 2. EGFP Transgene Expression is Severely Reduced in Mouse Inner Ear Tissue when Under the Control of the Murine SLC26A4 Core Promoter

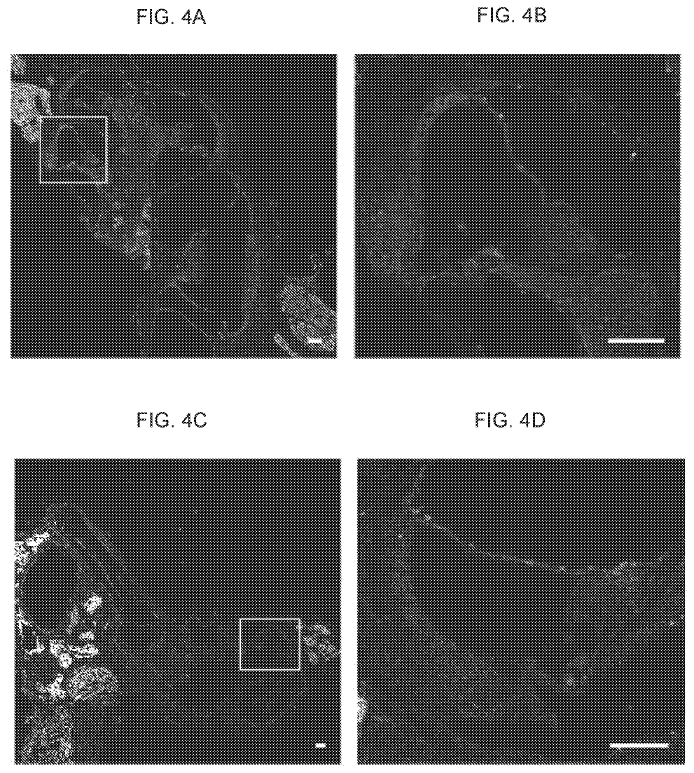

An in vivo experiment was conducted to assess whether expression of the AAV1 EGFP transgene could be restricted to SLC26A4-expressing cells of the cochlea and vestibule by replacing the ubiquitous CMV promoter with the murineSLC26A4 core promoter. To test this, a plasmid containing an expression cassette encoding the murine SLC26A4 core promoter (SEQ ID NO: 1) without any enhancer sequences driving expression of a nuclear-targeted EGFP was packaged into AAV1 at a titer of 6.28×10¹³ vg/mL. The resulting AAV virus was administered locally to adult C57BL/6 mice via the posterior semicircular canal (IL) at a dose of 6.28×10¹⁰ vg/ear. Two weeks after administration of the virus, whole ears were fixed, decalcified, and paraffin-embedded, and sections were imaged for EGFP using fluorescence microscopy. EGFP fluorescence was not detectable in any cell types in the cochlea (FIGS. 4A-4D) or vestibule (data not shown). The fluorescence observed in the wider views in FIGS. 4A and 4C is unrelated to EGFP expression and is the result of naturally fluorescing structures in the mouse ear that are observable when the intensity of display is raised significantly, as it was here in order to detect any EGFP-specific staining.

### Example 3. Addition of Enhancers to the Murine SLC26A4 Core Promoter Resulted in Increased EGFP Transgene Expression in Target Cell Types within the Cochlea and Vestibule

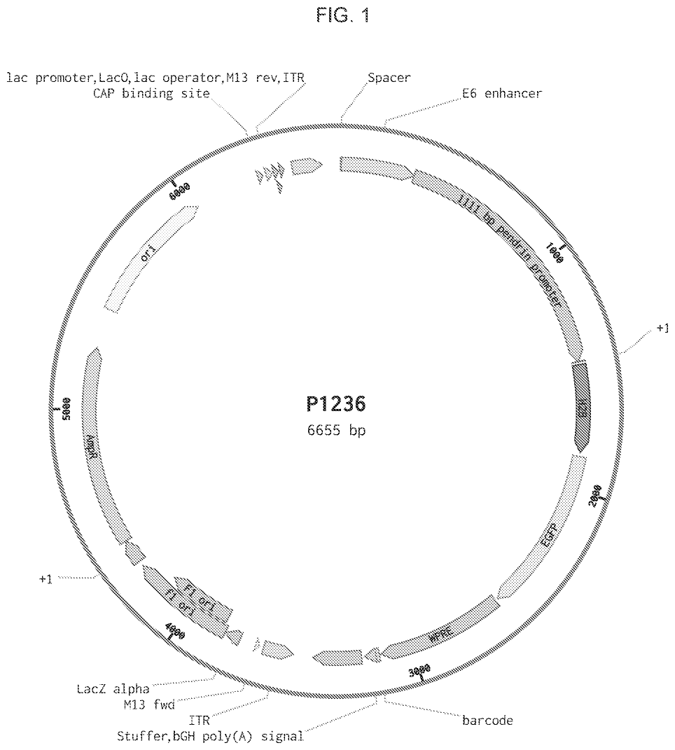

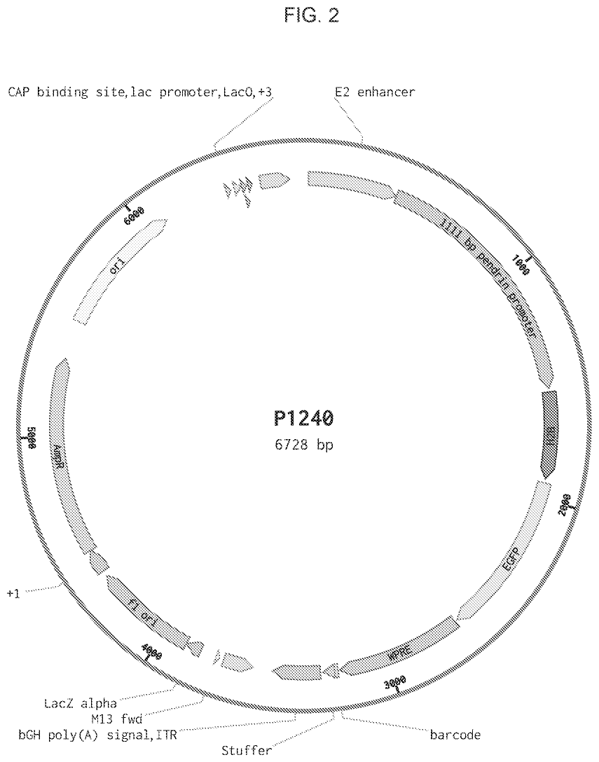

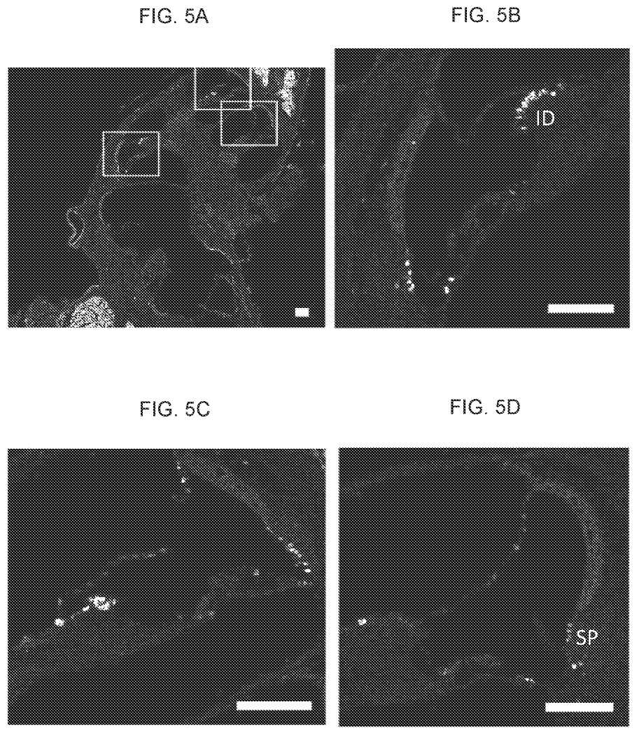

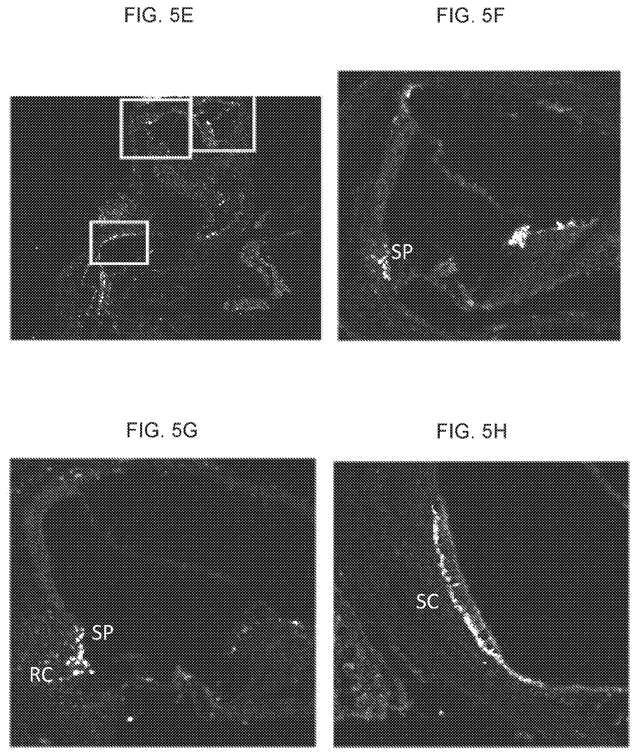

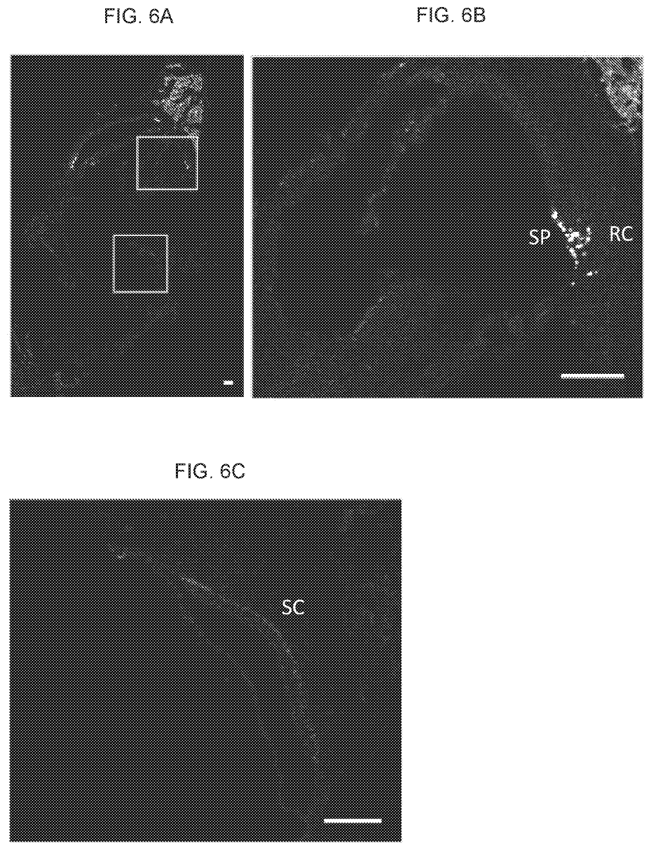

To test whether the combination of the murine SLC26A4 core promoter with enhancer element(s) would increase EGFP expression in target cell types within both the cochlea and vestibule, plasmids containing an expression cassette encoding the E2 enhancer (SEQ ID NO: 2) fused directly to the 5′ end of the murine SLC26A4 core promoter (SEQ ID NO: 1) (plasmid P1240; FIG. 2), or the E6 enhancer (SEQ ID NO: 3) fused directly to the 5′ end of the murine SLC26A4 core promoter (SEQ ID NO: 1) (plasmid P1236; FIG. 1) driving expression of a nuclear-targeted EGFP were packaged separately into AAV1 at a titer of 7.12×10¹³ and 6.68×10¹³, respectively. Each of these AAV1 vectors were separately administered locally to different adult C57BL/6 mice via the posterior semicircular canal (IL) at a dose of 7.12×10¹⁰ and 6.68×10¹⁰ vg/ear, respectively. Two weeks after administration of the virus, whole ears were fixed, decalcified, and paraffin-embedded, and sections were imaged for EGFP using fluorescence microscopy. In ears treated with AAV1 containing the E2 enhancer, EGFP fluorescence was detected in the nuclei of interdental cells (ID), spiral prominence cells (SP), and root cells (RC) of the cochlea (FIGS. 5A-5G) as well as supporting cells of the vestibular otolith organs (FIGS. 5E and 5H). In ears treated with AAV1 containing the E6 enhancer, EGFP fluorescence was detected in the nuclei of spiral prominence cells (SP) and root cells (RC) of the cochlea (FIGS. 6A-6B). Weak EGFP fluorescent signal was detected in the supporting cells (SC) of the vestibular otolith organs (FIGS. 6A and 6C).

### Example 4. The Presence of Multiple Enhancers in an AAV1 Vector Increases SLC26A4 Promoter-Driven GFP Expression in Mouse Cochlear Lateral Wall Explants without Affecting Cell-Specificity

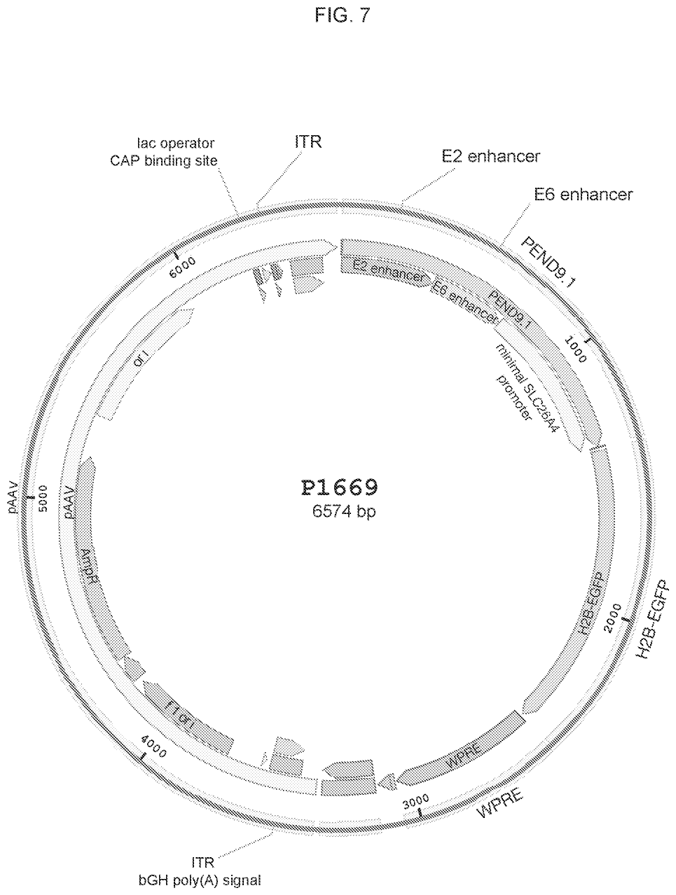

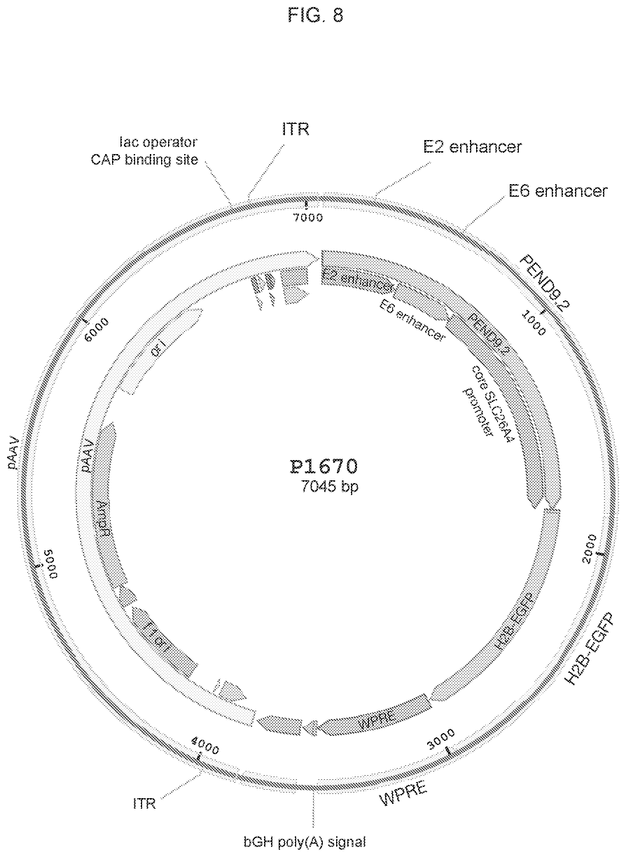

The lateral wall from cochleae of 6-8 week-old male C57BL/6J mice (000664, The Jackson Laboratory) were excised for culture with AAV1 viral vectors derived from transgene plasmids containing a nucleus-directed H2B-EGFP fusion transgene driven by a variety of promoters (CMV and a variety of SLC26A4 promoters described herein without an enhancer), as well as AAV1 vectors containing a H2B-EGFP fusion transgene driven by the murine core SLC26A4 promoter (SEQ ID NO: 1) and both the murine E2 enhancer (SEQ ID NO:2) and the murine E6 enhancer (SEQ ID NO:3) (P1670; FIG. 8); or the murine minimal SLC26A4 promoter (SEQ ID NO: 17) and both the murine E2 enhancer and the murine E6 enhancer (P1669; FIG. 7). After animals were sacrificed by CO₂ euthanasia, temporal bones were harvested, and the cochlear lateral wall was dissected in ice-cold DMEM/F-12 solution (11039021, Gibco) and cultured in DMEM/F-12, GlutaMax culture media (10565018, Gibco) supplemented with 10% FBS (F4135, Sigma) and 10 μg/ml Ciprofloxacin (AC456880050, Fisher Scientific) in glass bottom culture dishes (10810-054, Matsunami Glass). AAV were added to the media in 250 μl of culture media and were left in the culture media for 3 days before washing out with 2 ml of fresh media formulated as described above. The cultured lateral walls were then kept in culture for an additional 2 days (for 5 days total in culture after adding AAV to the media).

At the end of the culture, samples were fixed with fresh 4% formaldehyde in 1×PBS for 1 hour at room temperature (RT) and rinsed with 1×PBS for 3 times, 5 minutes each. Tissues were blocked with 10% normal donkey serum, 0.5% TritonX-100, in PBS at pH 7.4 for 1 hour at room temperature, followed by incubation with primary antibody against pendrin (BiCell Scientific 20501) diluted 1:100 with 0.5% TritonX-100 in 1×PBS overnight at 4° C. The next day, after washing with PBS (3 times, 5 min.), tissues were incubated with secondary antibody (1:500; Invitrogen A10042: Donkey (host), Rabbit IgG (target species) coupled to Alexa Fluor 568) for 2 hours at room temperature. After secondary antibody incubation, tissues were washed with PBS (3×, 5 mins.) and then were mounted in Slowfade Diamond Antifade Mounting Media (DAKO) (ThermoFisher Molecular probes, s36963).

After mounting, lateral walls were imaged using the Zeiss LSM 880 confocal microscope on both the 488 (EGFP) and 568 (pendrin) channels. Laser power and gain were set to achieve the highest EGFP signal without saturation of the detector. After establishing the imaging settings, all groups were imaged with the same laser power and gain within a study to allow for comparison between groups.

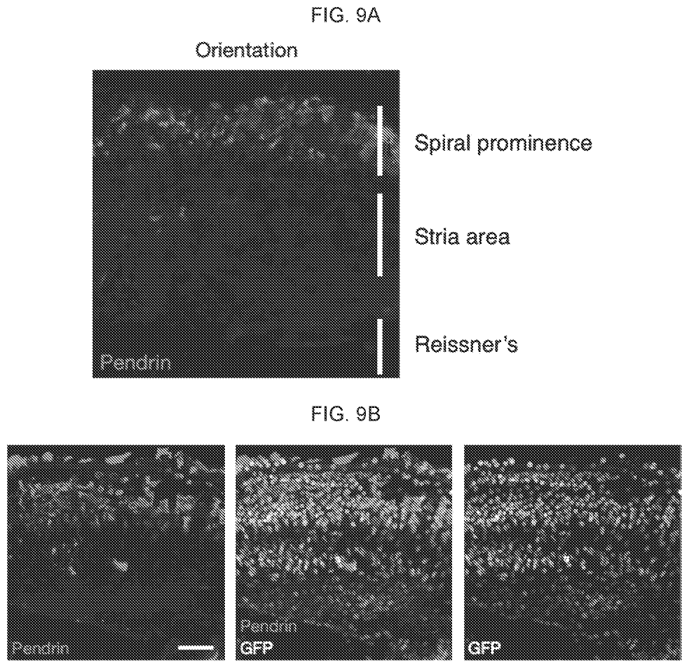

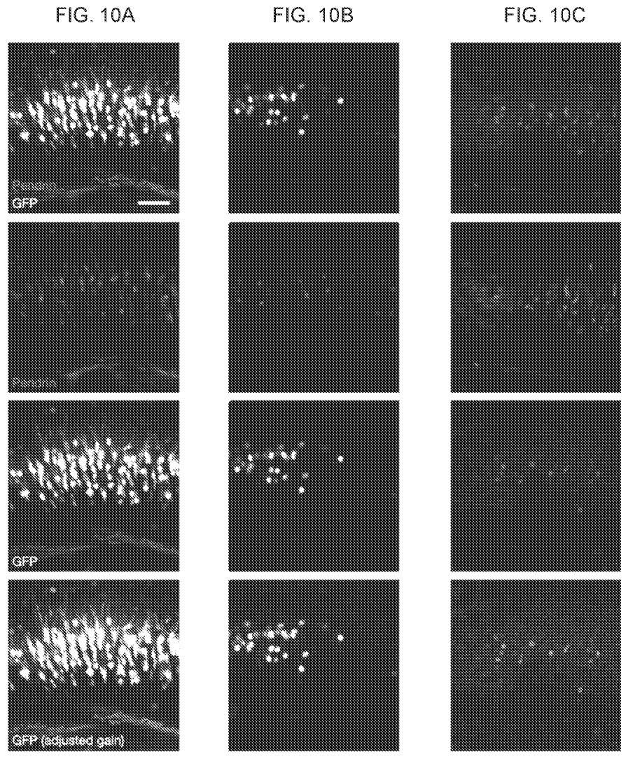

As can be seen from FIG. 9A, most of the SLC26A4-expressing cells are present in the spiral prominence and tight junctions of the stria vascularis. AAV expressing EGFP under control of the ubiquitous CMV promoter produced EGFP expression throughout the lateral wall that was not limited to the SLC26A4-expressing regions, demonstrating the ubiquity of AAV1 tropism in the lateral wall with that promoter (FIG. 9B). Replacing the CMV promoter with the murine core or minimal promoter, combined with the E2 and/or E6 enhancers disclosed herein, resulted in restriction of expression of EGFP mostly to cells that also express pendrin, with little or no EGFP expression in other cells of the lateral wall (FIGS. 10A-10C).

The inclusion of both the murine E6 and E2 SLC26A4 enhancer sequences fused to the 5′ end of the minimal or core murine promoter in AAV vectors expressing EGFP caused expression of EGFP without a loss of specificity to SLC26A4-expressing cells (FIGS. 10A and 101B). Interestingly, when both the murine E2 and murineE6 enhancers were coupled to either the murine minimal promoter (FIG. 10A) or murine core promoter (FIG. 101B) both resulted in specific expression of EGFP that was stronger than with only the murine E2 enhancer coupled to the murine core promoter (FIG. 10C).

### Example 5. In Vivo Administration of an AAV1 Vector Containing Multiple Enhancers Increases SLC26A4 Promoter-Driven GFP Expression in the Mouse Cochlea

To test whether combining both the E2 and E6 enhancers would further increase EGFP expression in vivo as shown in Example 3, plasmids containing an EGFP expression cassette encoding the E2 enhancer (SEQ ID NO: 2) fused directly to the 5′ end of the E6 enhancer (SEQ ID NO: 3) fused directly to the 5′ end of either the murine SLC26A4 core promoter (SEQ ID NO: 1) (plasmid P1670; FIG. 8), or the murine SLC26A4 minimal promoter (SEQ ID NO: 17) (plasmid P1669; FIG. 7) were packaged into AAV1 at titers of 4.41×10¹³ (lot 1), 2.70×10¹³ (lot 2), and 4.60×10¹³ vg/mL, respectively.

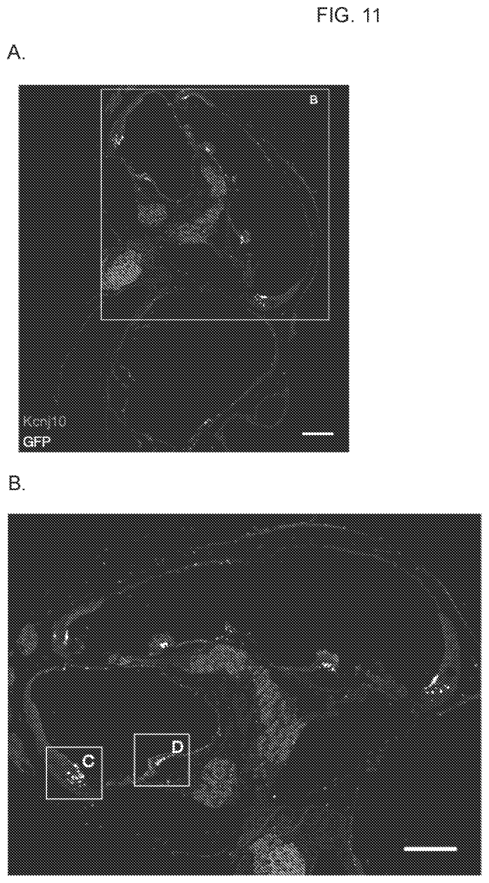

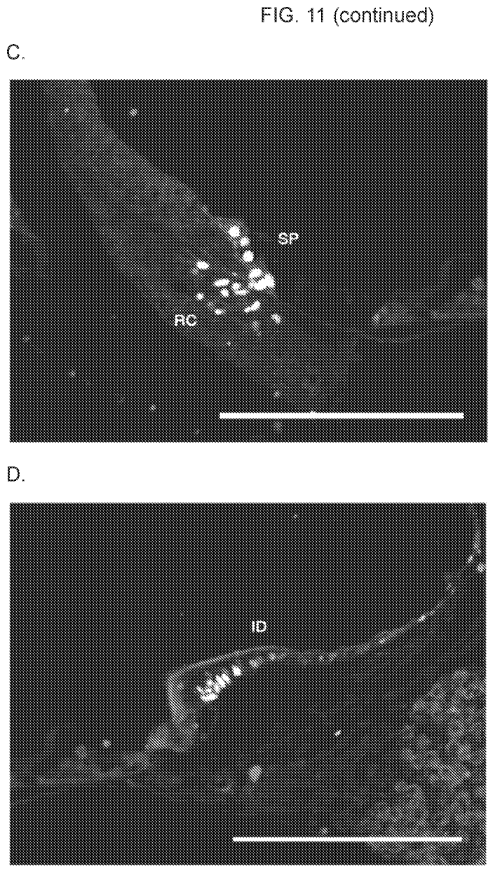

Lot 2 of the AAV1 vector with the minimal SLC26A4 promoter containing an expression cassette encoding the E2 enhancer (SEQ ID NO: 2) and the E6 enhancer (SEQ ID NO: 3) was administered locally to neonatal pendrin knockout mice at ages of P1-P3 via the posterior semicircular canal (IL) bilaterally at a dose of 1.5×10¹⁰ gc/ear. These pendrin KO mice were generated by CRISPR/Cas9-mediated deletion of exons 3-5 of the SLC26A4 gene on mouse chromosome 12 from mice with a C57BL/6 background. At the age of P21, whole ears were fixed, decalcified, and paraffin-embedded, and sections were imaged for EGFP using fluorescence microscopy. Sections were additionally stained for Kcnj10 (Abnova H00003766-M01) to visualize cochlear structures. In ears treated with AAV1 containing the coupled E2 and E6 enhancers with the minimal promoter, EGFP fluorescence was detected in the nuclei of interdental cells (ID), spiral prominence cells (SP), and root cells (RC) of the cochlea (FIG. 11).

### Example 6. In Vivo Administration of an AAV1 Vector Containing Multiple SLC26A4 Enhancers and an SLC26A4 Promoter Induces GFP Expression in SLC26A4-Expressing Cells in the Cochlea of Non-Human Primates

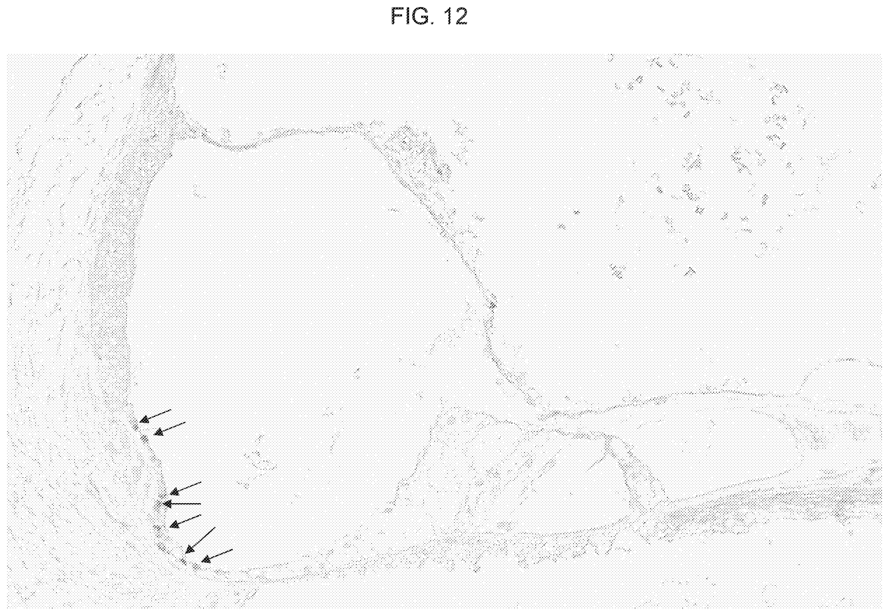

Plasmid containing the EGFP expression cassette encoding the E2 enhancer (SEQ ID NO: 2) fused directly to the 5′ end of the E6 enhancer (SEQ ID NO: 3) fused directly to the 5′ end of the murine SLC26A4 minimal promoter (SEQ ID NO: 17) (plasmid P1669) was packaged into AAV. The virus was administered locally to 2-4-year-old Cynomolgus monkeys (Macaca fascicularis). Administration was performed bilaterally by first creating a fenestration of the PSCC to allow for fluid egress followed by delivery of 60 μL/ear of virus via the round window membrane. After 2-weeks in life, animals were perfused, whole ears were collected, decalcified, paraffin-embedded, and sections were imaged for EGFP using fluorescence microscopy. In ears treated with AAV1 containing the coupled E2 and E6 enhancers with the minimal promoter, EGFP fluorescence was detected in the nuclei of spiral prominence cells (SP), root cells (RC), and outer sulcus cells of the cochlea (FIG. 12).

### Example 7. Administration of a Composition Containing a Nucleic Acid Vector Containing an SLC26A4 Enhancer and an SLC26A4 Promoter to a Subject with Sensorineural Hearing Loss

According to the methods disclosed herein, a physician of skill in the art can treat a patient, such as a human patient, with hearing loss (e.g., pendrin-related hearing loss, such as DFNB4 or Pendred syndrome) so as to improve or restore hearing. To this end, a physician of skill in the art can administer to the human patient a composition containing an AAV vector (e.g., an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ, DJ/8, DJ/9, 7m8, PHP.B, PHP.eB, or PHP.S vector) containing at least one SLC26A4 enhancer described herein (e.g., one or more copies of a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 and/or SEQ ID NO: 3) operably linked to an SLC26A4 promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17) that is operably linked to a polynucleotide encoding an expression product (e.g., a wild-type version of pendrin, such as a polynucleotide encoding SEQ ID NO: 4 or SEQ ID NO: 5). The composition containing the AAV vector may be administered to the patient, for example, by local administration to the inner ear (e.g., injection into the perilymph or endolymph or through the round window membrane) to treat sensorineural hearing loss.

Following administration of the composition to a patient, a practitioner of skill in the art can monitor the patient's improvement in response to the therapy by a variety of methods. For example, a physician can monitor the patient's hearing by performing standard tests, such as audiometry, ABR, electrocochleography (ECOG), and otoacoustic emissions following administration of the composition. A finding that the patient exhibits improved hearing in one or more of the tests following administration of the composition compared to hearing test results prior to administration of the composition indicates that the patient is responding favorably to the treatment. Subsequent doses can be determined and administered as needed.

### Example 8. Administration of a Composition Containing a Nucleic Acid Vector Containing an SLC26A4 Enhancer and an SLC26A4 Promoter to a Subject with Vestibular Dysfunction

According to the methods disclosed herein, a physician of skill in the art can treat a patient, such as a human patient, with vestibular dysfunction (e.g., vestibular dysfunction associated with loss of hair cells, such as age-related vestibular dysfunction or ototoxic drug-induced vestibular dysfunction) so as to improve or restore vestibular function. To this end, a physician of skill in the art can administer to the human patient a composition containing an AAV vector (e.g., an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ, DJ/8, DJ/9, 7m8, PHP.B, PHP.eB, or PHP.S vector) containing at least one SLC26A4 enhancer described herein (e.g., one or more copies of a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 and/or SEQ ID NO: 3) operably linked to an SLC26A4 promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17) that is operably linked to a polynucleotide encoding an expression product (e.g., a polynucleotide encoding a wild-type form of Atoh1, such as a polynucleotide encoding SEQ ID NO: 8 or SEQ ID NO: 10). The composition containing the AAV vector may be administered to the patient, for example, by local administration to the inner ear (e.g., injection into the perilymph or endolymph, through the round window membrane, or into a semicircular canal) to treat vestibular dysfunction.

Following administration of the composition to a patient, a practitioner of skill in the art can monitor the expression of the therapeutic protein encoded by the transgene, and the patient's improvement in response to the therapy, by a variety of methods. For example, a physician can monitor the patient's vestibular function by performing standard tests such as electronystagmography, video nystagmography, VOR tests (e.g., head impulse tests (Halmagyi-Curthoys test, e.g., VHIT), or caloric reflex tests), rotation tests, vestibular evoked myogenic potential, or computerized dynamic posturography. A finding that the patient exhibits improved vestibular function in one or more of the tests following administration of the composition compared to test results obtained prior to administration of the composition indicates that the patient is responding favorably to the treatment. Subsequent doses can be determined and administered as needed.

### Example 9. Administration of a Composition Containing a Nucleic Acid Vector Containing an SLC26A4 Enhancer and an SLC26A4 Promoter to a Subject with Meniere's Disease

According to the methods disclosed herein, a physician of skill in the art can treat a patient, such as a human patient, with Meniere's disease so as to reduce vertigo, improve hearing, reduce tinnitus, or reduce a sensation of fullness in the ear. To this end, a physician of skill in the art can administer to the human patient a composition containing an AAV vector (e.g., an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ, DJ/8, DJ/9, 7m8, PHP.B, PHP.eB, or PHP.S vector) containing at least one SLC26A4 enhancer described herein (e.g., one or more copies of a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 and/or SEQ ID NO: 3) operably linked to an SLC26A4 promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17) that is operably linked to a polynucleotide encoding an expression product (e.g., a wild-type version of pendrin, such as a polynucleotide encoding SEQ ID NO: 4 or SEQ ID NO: 5). The composition containing the AAV vector may be administered to the patient, for example, by local administration to the inner ear (e.g., injection into the perilymph or endolymph, through the round window membrane, or into a semicircular canal) to treat Meniere's disease.

Following administration of the composition to a patient, a practitioner of skill in the art can monitor the expression of the therapeutic protein encoded by the transgene, and the patient's improvement in response to the therapy, by a variety of methods. For example, a physician can monitor the patient's vestibular function by performing standard tests such as electronystagmography, video nystagmography, VOR tests (e.g., head impulse tests (Halmagyi-Curthoys test, e.g., VHIT), or caloric reflex tests), rotation tests, vestibular evoked myogenic potential, or computerized dynamic posturography; and can monitor the patient's hearing by performing standard tests, such as audiometry, ABR, electrocochleography (ECOG), and otoacoustic emissions following administration of the composition. A physician can also rely on patient reports regarding vertigo, tinnitus, and the sensation of fullness in the ear. A finding that the patient exhibits improved vestibular function or hearing in one or more of the tests or reports reduced vertigo, tinnitus, or a reduced sensation of fullness in the ear following administration of the composition compared to test results obtained prior to administration of the composition indicates that the patient is responding favorably to the treatment. Subsequent doses can be determined and administered as needed.

Exemplary embodiments of the invention are described in the enumerated paragraphs below.
E1. A polynucleotide comprising an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 3 operably linked to a promoter, wherein when the distance between the enhancer and the promoter in the polynucleotide is less than 3 kilobases (3 kb) (e.g., about 3.0 kb, 2.75 kb, 2.5 kb, 2.25 kb, 2.0 kb, 1.75 kb, 1.5 kb, 1.25 kb, 1.0 kb, 900 bases, 800 bases, 700 bases, 600 bases, 500 bases, 400 bases, 300 bases, 200 bases, 100 bases, 50 bases, or less).
E2. The polynucleotide of E1, wherein the distance between the enhancer and the promoter in the polynucleotide is less than 2 kb.
E3. The polynucleotide of E2, wherein the distance between the enhancer and the promoter in the polynucleotide is less than 1 kb.
E4. The polynucleotide of E3, wherein the distance between the enhancer and the promoter in the polynucleotide is less than 0.5 kb.
E5. The polynucleotide of E4, wherein the distance between the enhancer and the promoter in the polynucleotide is less than 100 bases.
E6. The polynucleotide of any one of E1-E5, wherein the enhancer is positioned 5′ of the promoter in the polynucleotide.
E7. The polynucleotide of any one of E1-E5, wherein the enhancer is positioned 3′ of the promoter in the polynucleotide.
E8. The polynucleotide of any one of E1-E7, wherein the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2.
E9. The polynucleotide of E8, wherein the enhancer has the sequence of SEQ ID NO: 2.
E10. The polynucleotide of any one of E1-E7, wherein the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 3.
E11. The polynucleotide of E10, wherein the enhancer has the sequence of SEQ ID NO: 3.
E12. The polynucleotide of any one of E1-E11, wherein the enhancer is directly fused to the promoter.
E13. The polynucleotide of any one of E1-E11, wherein the enhancer is joined to the promoter by a nucleic acid linker of one to 100 nucleic acids (e.g., about 1, 5, 10, 15, 20, 30, 40, 50, 60, 70, 80, 90, or 100 nucleic acids).
E14. The polynucleotide of any one of E1-E13, wherein the promoter is a minimal promoter, a core promoter, or a constitutive promoter.
E15. The polynucleotide of E14, wherein the promoter is a CAG promoter, a CBA promoter, an smCBA promoter, a CASI promoter, a dihydrofolate reductase (DHFR) promoter, a β-actin promoter, a phosphoglycerol kinase (PGK) promoter, an EF1α promoter, a β-globin promoter, a CMV promoter, an HSV promoter, or an SV40 promoter.
E16. The polynucleotide of E15, wherein the promoter is a minimal β-globin promoter, a CMVmini promoter, a minCMV promoter, a CMV-TATA+INR promoter, a min CMV-T6 promoter, a minimal HSV ICP0 promoter, a truncated HSV ICP0 promoter, or an SV40 minimal promoter.
E17. The polynucleotide of any one of E1-E13, wherein the promoter is a minimal promoter.
E18. The polynucleotide of any one of E1-E13, wherein the promoter is a mammalian SLC26A4 promoter.
E19. The polynucleotide of E18, wherein the SLC26A4 promoter is a human or murine SLC26A4 promoter.
E20. The polynucleotide of E19, wherein the SLC26A4 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1.
E21. The polynucleotide of E20, wherein the SLC26A4 promoter has the sequence of SEQ ID NO: 1.
E22. The polynucleotide of E19, wherein the SLC26A4 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 12-16.
E23. The polynucleotide of E22, wherein the SLC26A4 promoter has the sequence of any one of SEQ ID NOs: 12-16.
E24. The polynucleotide of E19, wherein the SLC26A4 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17.
E25. The polynucleotide of E24, wherein the SLC26A4 promoter has the sequence of SEQ ID NO: 17.
E26. The polynucleotide of any one of E1-E25, wherein the promoter is operably linked to a polynucleotide that can be transcribed to produce an expression product.
E27. The polynucleotide of E26, wherein the expression product is a heterologous expression product.
E28. The polynucleotide of E26, wherein the expression product is an expression product that is endogenously expressed in a SLC26A4-expressing cell.
E29. The polynucleotide of E28, wherein the expression product is an expression product that is endogenously expressed in a SLC26A4-expressing inner ear cell.
E30. The polynucleotide of E29, wherein the expression product is an expression product that is endogenously expressed in an interdental cell, a spiral prominence cell, a cochlear root cell, and/or a vestibular supporting cell (e.g., expressed in at least one of these cell types).
E31. The polynucleotide of any one of E28-E30, wherein the expression product is pendrin (e.g., a mammalian pendrin protein).
E32. The polynucleotide of E31, wherein the pendrin (the mammalian pendrin protein) is a wild-type isoform endogenously expressed in an inner ear of a mammal.
E33. The polynucleotide of any one of E31 or E32, wherein the pendrin (the mammalian pendrin protein) has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 4 or SEQ ID NO: 5.
E34. The polynucleotide of E33, wherein the pendrin (the mammalian pendrin protein) has the sequence of SEQ ID NO: 4 or SEQ ID NO: 5.
E35. The polynucleotide of any one of E27-E30, wherein the expression product is Atoh1 (e.g., mammalian Atoh1).
E36. The polynucleotide of E35, wherein the Atoh1 (the mammalian Atoh1 protein) is a wild-type isoform endogenously expressed in an inner ear of a mammal.
E37. The polynucleotide of any one of E35 or E36, wherein the Atoh1 (the mammalian Atoh1 protein) has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 8 or SEQ ID NO: 10.
E38. The polynucleotide of E37, wherein the Atoh1 (the mammalian Atoh1 protein) has the sequence of SEQ ID NO: 8 or SEQ ID NO: 10.
E39. The polynucleotide of E26 or E27, wherein the expression product is a protein, a short hairpin RNA (shRNA), an antisense oligonucleotide (ASO), a component of a gene editing system (e.g., a nuclease, such as a CRISPR Associated Protein 9 (Cas9), Transcription Activator-Like Effector Nuclease (TALEN), or Zinc Finger Nuclease (ZFN), or a guide RNA (gRNA)), or a microRNA.
E40. The polynucleotide of any one of E1-E39, wherein the polynucleotide comprises an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 and an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 3.
E41. The polynucleotide of E40, wherein the polynucleotide comprises an enhancer that has the sequence of SEQ ID NO: 2 and an enhancer that has the sequence of SEQ ID NO: 3.
E42. The polynucleotide of E41, wherein the polynucleotide comprises, in 5′ to 3′ order, an enhancer having the sequence of SEQ ID NO: 2, an enhancer having the sequence of SEQ ID NO: 3, and a SLC26A4 promoter having the sequence of SEQ ID NO: 1 or SEQ ID NO: 17 (e.g., an enhancer having the sequence of SEQ ID NO: 2 directly fused to an enhancer having the sequence of SEQ ID NO: 3 directly fused to a SLC26A4 promoter having the sequence of SEQ ID NO: 1 or SEQ ID NO: 17).
E43. The polynucleotide of E42, wherein the SLC26A4 promoter has the sequence of SEQ ID NO: 17.
E44. The polynucleotide of E42 or E43, wherein the polynucleotide comprises the sequence of SEQ ID NO: 18.
E45. The polynucleotide of any one of E1-E41, wherein the polynucleotide comprises two or more copies of an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 and/or two or more copies of an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) SEQ ID NO: 3 (e.g., two or more copies of one or both enhancers).
E46. The polynucleotide of E45, wherein the polynucleotide comprises two or more copies of an enhancer that has the sequence of SEQ ID NO: 2 and/or SEQ ID NO: 3 (e.g., two or more copies of one or both enhancers).
E47. A nucleic acid vector comprising the polynucleotide of any one of E1-E46.
E48. A nucleic acid vector comprising a polynucleotide comprising an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 3.
E49. The nucleic acid vector of E48, wherein the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2.
E50. The nucleic acid vector of E49, wherein the enhancer has the sequence of SEQ ID NO: 2.
E51. The nucleic acid vector of E48, wherein the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 3.
E52. The nucleic acid vector of E51, wherein the enhancer has the sequence of SEQ ID NO: 3.
E53. The nucleic acid vector of any one of E48-E52, wherein the enhancer is operably linked to a promoter.
E54. The nucleic acid vector of E53, wherein the enhancer is positioned 5′ of the promoter.
E55. The nucleic acid vector of E53, wherein the enhancer is positioned 3′ of the promoter.
E56. The nucleic acid vector of any one of E53-E55, wherein the enhancer is directly fused to the promoter.
E57. The nucleic acid vector of any one of E53-E55, wherein the enhancer is joined to the promoter by a nucleic acid linker of one to 100 nucleic acids (e.g., about 1, 5, 10, 15, 20, 30, 40, 50, 60, 70, 80, 90, or 100 nucleic acids).
E58. The nucleic acid vector of any one of E53-E57, wherein the promoter is a minimal promoter, a core promoter, or a constitutive promoter.
E59. The nucleic acid vector of E58, wherein the promoter is a CAG promoter, a CBA promoter, an smCBA promoter, a CASI promoter, a dihydrofolate reductase (DHFR) promoter, a β-actin promoter, a phosphoglycerol kinase (PGK) promoter, an EF1α promoter, a β-globin promoter, a CMV promoter, an HSV promoter, or an SV40 promoter.
E60. The nucleic acid vector of E59, wherein the promoter is a minimal β-globin promoter, a CMVmini promoter, a minCMV promoter, a CMV-TATA+INR promoter, a min CMV-T6 promoter, a minimal HSV ICP0 promoter, a truncated HSV ICP0 promoter, or an SV40 minimal promoter.
E61. The nucleic acid vector of E58, wherein the promoter is a minimal promoter.
E62. The nucleic acid vector of any one of E53-E57, wherein the promoter is a mammalian SLC26A4 promoter.
E63. The nucleic acid vector of E62, wherein the SLC26A4 promoter is a human or murine SLC26A4 promoter.
E64. The nucleic acid vector of E63, wherein the SLC26A4 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1.
E65. The nucleic acid vector of E64, wherein the SLC26A4 promoter has the sequence of SEQ ID NO: 1.
E66. The nucleic acid vector of E63, wherein the SLC26A4 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 12-16.
E67. The nucleic acid vector of E66, wherein the SLC26A4 promoter has the sequence of any one of SEQ ID NOs: 12-16.
E68. The nucleic acid vector of E63, wherein the SLC26A4 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17.
E69. The nucleic acid vector of E68, wherein the SLC26A4 promoter has the sequence of SEQ ID NO: 17.
E70. The nucleic acid vector of any one of E53-E69, wherein the promoter is operably linked to a polynucleotide that can be transcribed to produce an expression product.
E71. The nucleic acid vector of E70, wherein the expression product is a heterologous expression product.
E72. The nucleic acid vector of E70, wherein the expression product is an expression product that is endogenously expressed in an SLC26A4-expressing cell.
E73. The nucleic acid vector of E72, wherein the expression product is an expression product that is endogenously expressed in an SLC26A4-expressing inner ear cell.
E74. The nucleic acid vector of E73, wherein the expression product is an expression product that is endogenously expressed in an interdental cell, a spiral prominence cell, a cochlear root cell, and/or a vestibular supporting cell (e.g., expressed in at least one of these cell types).
E75. The nucleic acid vector of any one of E72-E74, wherein the expression product is pendrin (e.g., a mammalian pendrin protein).
E76. The nucleic acid vector of E75, wherein the pendrin (the mammalian pendrin protein) is a wild-type isoform endogenously expressed in an inner ear of a mammal.
E77. The nucleic acid vector of E75 or E76, wherein the pendrin (the mammalian pendrin protein) has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 4 or SEQ ID NO: 5.
E78. The nucleic acid vector of E77, wherein the pendrin (the mammalian pendrin protein) has the sequence of SEQ ID NO: 4 or SEQ ID NO: 5.
E79. The nucleic acid vector of any one of E71-E74, wherein the expression product is Atoh1 (e.g., a mammalian Atoh1 protein).
E80. The nucleic acid vector of E79, wherein the Atoh1 (the mammalian Atoh1 protein) is a wild-type isoform endogenously expressed in an inner ear of a mammal.
E81. The nucleic acid vector of any one of E79 or E80, wherein the Atoh1 (the mammalian Atoh1 protein) has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 8 or SEQ ID NO: 10.
E82. The nucleic acid vector of E81, wherein the Atoh1 (the mammalian Atoh1 protein) has the sequence of SEQ ID NO: 8 or SEQ ID NO: 10.
E83. The nucleic acid vector of E70 or E71, wherein the expression product is a protein, a short hairpin RNA (shRNA), an antisense oligonucleotide (ASO), a component of a gene editing system (e.g., a nuclease, such as a CRISPR Associated Protein 9 (Cas9), Transcription Activator-Like Effector Nuclease (TALEN), or Zinc Finger Nuclease (ZFN), or a guide RNA (gRNA)), or a microRNA.
E84. The nucleic acid vector of any one of E48-E83, wherein the polynucleotide comprises an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 and an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 3.
E85. The nucleic acid vector of E84, wherein the polynucleotide comprises an enhancer that has the sequence of SEQ ID NO: 2 and an enhancer that has the sequence of SEQ ID NO: 3.
E86. The nucleic acid vector of E85, wherein the polynucleotide comprises, in 5 to 3′ order, an enhancer having the sequence of SEQ ID NO: 2, an enhancer having the sequence of SEQ ID NO: 3, and a SLC26A4 promoter having the sequence of SEQ ID NO: 1 or SEQ ID NO: 17 (e.g., an enhancer having the sequence of SEQ ID NO: 2 fused directly to an enhancer having the sequence of SEQ ID NO: 3 fused directly to a SLC26A4 promoter having the sequence of SEQ ID NO: 1 or SEQ ID NO: 17).
E87. The nucleic acid vector of E86, wherein the SLC26A4 promoter has the sequence of SEQ ID NO: 17.
E88. The nucleic acid vector of E86 or E87, wherein the polynucleotide comprises the sequence of SEQ ID NO: 18.
E89. The nucleic acid vector of any one of E48-E85, wherein the polynucleotide comprises two or more copies of an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 and/or two or more copies of an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) SEQ ID NO: 3 (e.g., two or more copies of one or both enhancers).
E90. The nucleic acid vector of E89, wherein the polynucleotide comprises two or more copies of an enhancer that has the sequence of SEQ ID NO: 2 and/or SEQ ID NO: 3 (e.g., two or more copies of one or both enhancers).
E91. A nucleic acid vector comprising a polynucleotide comprising a SLC26A4 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1.
E92. The nucleic acid vector of E91, wherein the SLC26A4 promoter has the sequence of SEQ ID NO: 1.
E93. A nucleic acid vector comprising a polynucleotide comprising a SLC26A4 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17.
E94. The nucleic acid vector of E93, wherein the SLC26A4 promoter has the sequence of SEQ ID NO: 17.
E95. The nucleic acid vector of any one of E91-E94, wherein the promoter is operably linked to a polynucleotide that can be transcribed to produce an expression product.
E96. The nucleic acid vector of E95, wherein the expression product is a heterologous expression product.
E97. The nucleic acid vector of E95, wherein the expression product is an expression product that is endogenously expressed in a SLC26A4-expressing cell.
E98. The nucleic acid vector of E97, wherein the expression product is an expression product that is endogenously expressed in a SLC26A4-expressing inner ear cell.
E99. The nucleic acid vector of E98, wherein the expression product is an expression product that is endogenously expressed in an interdental cell, a spiral prominence cell, a cochlear root cell, and/or a vestibular supporting cell (e.g., expressed in at least one of these cell types).
E100. The nucleic acid vector of any one of E97-E99, wherein the expression product is pendrin (e.g., a mammalian pendrin protein).
E101. The nucleic acid vector of E100, wherein the pendrin (the mammalian pendrin protein) is a wild-type isoform endogenously expressed in an inner ear of a mammal.
E102. The nucleic acid vector of E100 or E101, wherein the pendrin (the mammalian pendrin protein) has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 4 or SEQ ID NO: 5.
E103. The nucleic acid vector of E102, wherein the pendrin (the mammalian pendrin protein) has the sequence of SEQ ID NO: 4 or SEQ ID NO: 5.
E104. The nucleic acid vector of any one of E95-E99, wherein the expression product is Atoh1 (e.g., a mammalian Atoh1 protein).
E105. The nucleic acid vector of E104, wherein the Atoh1 (the mammalian Atoh1 protein) is a wild-type isoform endogenously expressed in an inner ear of a mammal.
E106. The nucleic acid vector of E104 or E105, wherein the Atoh1 (the mammalian Atoh1 protein) has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 8 or SEQ ID NO: 10.
E107. The nucleic acid vector of E106, wherein the Atoh1 (the encoded mammalian Atoh1) has the sequence of SEQ ID NO: 8 or SEQ ID NO: 10.
E108. The nucleic acid vector of E95 or E96, wherein the expression product is a protein, a short hairpin RNA (shRNA), an antisense oligonucleotide (ASO), a component of a gene editing system (e.g., a nuclease, such as a CRISPR Associated Protein 9 (Cas9), Transcription Activator-Like Effector Nuclease (TALEN), or Zinc Finger Nuclease (ZFN), or a guide RNA (gRNA)), or a microRNA.
E109. The nucleic acid vector of any one of E91-E108, wherein the promoter is operably linked to an enhancer.
E110. The nucleic acid vector of E109, wherein the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or at least 85% sequence identity (e.g., 85%, 86%, 87%, 88%, 89%, 90%, 91%, 92%, 93%, 94%, 95%, 96%, 97%, 98%, 99%, or more, sequence identity) SEQ ID NO: 3.
E111. The nucleic acid vector of E110, wherein the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2.
E112. The nucleic acid vector of E111, wherein the enhancer has the sequence of SEQ ID NO: 2.
E113. The nucleic acid vector of E110, wherein the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 3.
E114. The nucleic acid vector of E113, wherein the enhancer has the sequence of SEQ ID NO: 3.
E115. The nucleic acid vector of any one of E109-E114, wherein the enhancer is positioned 5′ of the promoter.
E116. The nucleic acid vector of any one of E109-E114, wherein the enhancer is positioned 3′ of the promoter.
E117. The nucleic acid vector of any one of E109-E116, wherein the enhancer is directly fused to the promoter.
E118. The nucleic acid vector of any one of E109-E116, wherein the enhancer is joined to the promoter by a nucleic acid linker of one to 100 nucleic acids (e.g., about 1, 5, 10, 15, 20, 30, 40, 50, 60, 70, 80, 90, or 100 nucleic acids).
E119. The nucleic acid vector of any one of E109-E118, wherein the polynucleotide comprises an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 and an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 3.
E120. The nucleic acid vector of E119, wherein the polynucleotide comprises an enhancer that has the sequence of SEQ ID NO: 2 and an enhancer that has the sequence of SEQ ID NO: 3.
E121. The nucleic acid vector of E120, wherein the polynucleotide comprises, in 5′ to 3′ order, an enhancer having the sequence of SEQ ID NO: 2, an enhancer having the sequence of SEQ ID NO: 3, and a SLC26A4 promoter having the sequence of SEQ ID NO: 1 or SEQ ID NO: 17 (e.g., an enhancer having the sequence of SEQ ID NO: 2 fused directly to an enhancer having the sequence of SEQ ID NO: 3 fused directly to a SLC26A4 promoter having the sequence of SEQ ID NO: 1 or SEQ ID NO: 17).
E122. The nucleic acid vector of E121, wherein the SLC26A4 promoter has the sequence of SEQ ID NO: 17.
E123. The nucleic acid vector of E121 or E122, wherein the polynucleotide comprises the sequence of SEQ ID NO: 18.
E124. The nucleic acid vector of any one of E109-E120, wherein the polynucleotide comprises two or more copies of an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 and/or two or more copies of an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) SEQ ID NO: 3 (e.g., two or more copies of one or both enhancers).
E125. The nucleic acid vector of E1124, wherein the polynucleotide comprises two or more copies of an enhancer that has the sequence of SEQ ID NO: 2 and/or SEQ ID NO: 3 (e.g., two or more copies of one or both enhancers).
E126. A polynucleotide comprising a SLC26A4 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 operably linked to a polynucleotide that can be transcribed to produce an expression product.
E127. The polynucleotide of E126, wherein the SLC26A4 promoter has the sequence of SEQ ID NO: 1.
E128. A polynucleotide comprising a SLC26A4 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17 operably linked to a polynucleotide that can be transcribed to produce an expression product.
E129. The polynucleotide of E128, wherein the SLC26A4 promoter has the sequence of SEQ ID NO: 17.
E130. The polynucleotide of any one of E126-E129, wherein the expression product is a heterologous expression product.
E131. The polynucleotide of any one of E126-E129, wherein the expression product is an expression product that is endogenously expressed in a SLC26A4-expressing cell.
E132. The polynucleotide of E131, wherein the expression product is an expression product that is endogenously expressed in a SLC26A4-expressing inner ear cell.
E133. The polynucleotide of E132, wherein the expression product is an expression product that is endogenously expressed in an interdental cell, a spiral prominence cell, a cochlear root cell, and/or a vestibular supporting cell (e.g., expressed in at least one of these cell types).
E134. The polynucleotide of any one of E126-E129 and E131-E133, wherein the expression product is pendrin (e.g., a mammalian pendrin protein).
E135. The polynucleotide of E134, wherein the pendrin (the mammalian pendrin protein) is a wild-type isoform endogenously expressed in an inner ear of a mammal.
E136. The polynucleotide of E134 or E135, wherein the pendrin (the mammalian pendrin protein) has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 4 or SEQ ID NO: 5.
E137. The polynucleotide of E136, wherein the pendrin (the mammalian pendrin protein) has the sequence of SEQ ID NO: 4 or SEQ ID NO: 5.
E138. The polynucleotide of any one of E126-E133, wherein the expression product is Atoh1 (e.g., a mammalian Atoh1 protein).
E139. The polynucleotide of E138, wherein the Atoh1 (the mammalian Atoh1 protein) is a wild-type isoform endogenously expressed in an inner ear of a mammal.
E140. The polynucleotide of E138 or E139, wherein the Atoh1 (the mammalian Atoh1 protein) has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 8 or SEQ ID NO: 10.
E141. The polynucleotide of E140, wherein the Atoh1 (the encoded mammalian Atoh1) has the sequence of SEQ ID NO: 8 or SEQ ID NO: 10.
E142. The polynucleotide of any one of E126-E133, wherein the expression product is a protein, a short hairpin RNA (shRNA), an antisense oligonucleotide (ASO), a component of a gene editing system (e.g., a nuclease, such as a CRISPR Associated Protein 9 (Cas9), Transcription Activator-Like Effector Nuclease (TALEN), or Zinc Finger Nuclease (ZFN), or a guide RNA (gRNA)), or a microRNA.
E143. The polynucleotide of any one of E126-E142, wherein the promoter is operably linked to an enhancer.
E144. The polynucleotide of E143, wherein the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or at least 85% sequence identity (e.g., 85%, 86%, 87%, 88%, 89%, 90%, 91%, 92%, 93%, 94%, 95%, 96%, 97%, 98%, 99%, or more, sequence identity) SEQ ID NO: 3.
E145. The polynucleotide of E144, wherein the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2.
E146. The polynucleotide of E145, wherein the enhancer has the sequence of SEQ ID NO: 2.
E147. The polynucleotide of E144, wherein the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 3.
E148. The polynucleotide of E147, wherein the enhancer has the sequence of SEQ ID NO: 3.
E149. The polynucleotide of any one of E143-E148, wherein the enhancer is positioned 5′ of the promoter.
E150. The polynucleotide of any one of E143-E148, wherein the enhancer is positioned 3′ of the promoter.
E151. The polynucleotide of any one of E143-E150, wherein the enhancer is directly fused to the promoter.
E152. The polynucleotide of any one of E143-E150, wherein the enhancer is joined to the promoter by a nucleic acid linker of one to 100 nucleic acids (e.g., about 1, 5, 10, 15, 20, 30, 40, 50, 60, 70, 80, 90, or 100 nucleic acids).
E153. The polynucleotide of any one of E143-E152, wherein the polynucleotide comprises an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 and an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 3.
E154. The polynucleotide of E153, wherein the polynucleotide comprises an enhancer that has the sequence of SEQ ID NO: 2 and an enhancer that has the sequence of SEQ ID NO: 3.
E155. The polynucleotide of E154, wherein the polynucleotide comprises, in 5′ to 3′ order, an enhancer having the sequence of SEQ ID NO: 2, an enhancer having the sequence of SEQ ID NO: 3, and a SLC26A4 promoter having the sequence of SEQ ID NO: 1 or SEQ ID NO: 17 (e.g., an enhancer having the sequence of SEQ ID NO: 2 fused directly to an enhancer having the sequence of SEQ ID NO: 3 fused directly to a SLC26A4 promoter having the sequence of SEQ ID NO: 1 or SEQ ID NO: 17).
E156. The polynucleotide of E155, wherein the SLC26A4 promoter has the sequence of SEQ ID NO: 17.
E157. The polynucleotide of E155 or E156, wherein the polynucleotide comprises the sequence of SEQ ID NO: 18.
E158. The polynucleotide of any one of E143-E154, wherein the polynucleotide comprises two or more copies of an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 and/or two or more copies of an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) SEQ ID NO: 3 (e.g., two or more copies of one or both enhancers).
E159. The polynucleotide of E158, wherein the polynucleotide comprises two or more copies of an enhancer that has the sequence of SEQ ID NO: 2 and/or SEQ ID NO: 3 (e.g., two or more copies of one or both enhancers).
E160. A nucleic acid vector comprising the polynucleotide of any one of E126-E159.
E161. The nucleic acid vector of any one of E47-E125 and E160, wherein the nucleic acid vector is a viral vector, plasmid, cosmid, or artificial chromosome.
E162. The nucleic acid vector of any one of E47-E125, E160, and E161, wherein the nucleic acid vector is a viral vector.
E163. The nucleic acid vector of E162, wherein the viral vector is an adeno-associated virus (AAV) viral vector, an adenovirus viral vector, or a lentivirus viral vector.
E164. The nucleic acid vector of E163, wherein the viral vector is an AAV vector.
E165. The nucleic acid vector of E164, wherein the AAV vector has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ, DJ/8, DJ/9, 7m8, PHP.B, PHP.eB, or PHP.S capsid.
E166. A composition comprising the nucleic acid vector of any one of E47-E125 and E160-E165 and a pharmaceutically acceptable carrier, diluent, or excipient.
E167. A cell comprising the polynucleotide of any one of E1-E46 and E126-E159 or the nucleic acid vector of any one of E47-E125 and E160-E165.
E168. The cell of E167, wherein the cell is a SLC26A4-expressing cell.
E169. The cell of E168, wherein the cell is a SLC26A4-expressing inner ear cell.
E170. The cell of any one of E167-E169, wherein the cell is a mammalian cell.
E171. The cell of E170, wherein the mammalian cell is a human cell.
E172. The cell of any one of E167-E171, wherein the cell is an interdental cell, spiral prominence cell, cochlear root cell, or vestibular supporting cell.
E173. A method of expressing an expression product in a cell, the method comprising the step of contacting the cell with the nucleic acid vector of any one of E47-E125 and E160-E165 or the composition of E166.
E174. The method of E173, wherein the cell is an inner ear cell.
E175. The method of E173 or E174, wherein the cell is an SLC26A4-expressing cell.
E176. The method of any one of E173-E175, wherein the cell is an SLC26A4-expressing inner ear cell.
E177. The method of E176, wherein the SLC26A4-expressing inner ear cell is an interdental cell, spiral prominence cell, cochlear root cell, or vestibular supporting cell.
E178. The method of any one of E173-E177, wherein the cell is a mammalian cell.
E179. The method of E178, wherein the mammalian cell is a human cell.
E180. The method of any one of E173-E179, wherein the contacting is in a subject (e.g., in vivo).
E181. A method of treating a subject having or at risk of developing hearing loss (e.g., sensorineural hearing loss), the method comprising the step of administering to an inner ear of the subject a therapeutically effective amount of the nucleic acid vector of any one of E47-E125 and E160-E165 or the composition of E166.
E182. The method of E181, wherein the hearing loss is pendrin-related hearing loss.
E183. The method of E182, wherein the expression product is pendrin.
E184. The method of E182 or E183, wherein the pendrin-related hearing loss is hearing loss associated with Pendred syndrome or DFNB4.
E185. A method of treating hearing loss associated with Meniere's disease in a subject in need thereof, the method comprising the step of administering to an inner ear of the subject an effective amount of the nucleic acid vector of any one of E47-E125 and E160-E165 or the composition of E166.
E186. A method of treating tinnitus associated with Meniere's disease in a subject in need thereof, the method comprising the step of administering to an inner ear of the subject an effective amount of the nucleic acid vector of any one of E47-E125 and E160-E165 or the composition of E166.
E187. The method of E185 or E186, wherein the expression product is pendrin.
E188. A method of treating vestibular dysfunction associated with Meniere's disease in a subject in need thereof, the method comprising the step of administering to an inner ear of the subject an effective amount of the nucleic acid vector of any one of E47-E125 and E160-E165 or the composition of E166.
E189. The method of E188, wherein the expression product is pendrin or Atoh1.
E190. The method of E188 or E189, wherein the vestibular dysfunction is vertigo.
E191. A method of treating a subject having or at risk of developing vestibular dysfunction, the method comprising the step of administering to an inner ear of the subject a therapeutically effective amount of the nucleic acid vector of any one of E47-E125 and E160-E165 or the composition of E166.
E192. The method of E191, wherein the vestibular dysfunction is pendrin-related vestibular dysfunction.
E193. The method of E192, wherein the expression product is pendrin.
E194. The method of E192 or E193 wherein the pendrin-related vestibular dysfunction is vestibular dysfunction associated with Pendred syndrome or DFNB4.
E195. The method of E191, wherein the expression product is pendrin or Atoh1.
E196. A method of inducing or increasing differentiation of a vestibular supporting cell into a vestibular hair cell, the method comprising the step of contacting the vestibular supporting cell with the nucleic acid vector of any one of E47-E125 and E160-E165 or the composition of E166, wherein the expression product is Atoh1.
E197. The method of E196, wherein the contacting is in vivo (e.g., in a subject).
E198. The method of E197, wherein the subject has or is at risk of developing vestibular dysfunction.
E199. A method of inducing or increasing vestibular hair cell regeneration in a subject in need thereof, the method comprising the step of administering to an inner ear of the subject an effective amount of the nucleic acid vector of any one of E47-E125 and E160-E165 or the composition of E166, wherein the expression product is Atoh1.
E200. The method of E199, wherein the subject has or is at risk of developing vestibular dysfunction.
E201. A method of improving function of an SLC26A4-expressing cell, the method comprising the step of contacting the SLC26A4-expressing cell with the nucleic acid vector of any one of E47-E125 and E160-E165 or the composition of E166.
E202. The method of E201, wherein the contacting is in vivo (e.g., in a subject).
E203. The method of E202, wherein the subject has or is at risk of developing hearing loss (e.g., sensorineural hearing loss) or vestibular dysfunction.
E204. The method of any one of E191-E203, wherein the vestibular dysfunction is vertigo, dizziness, imbalance (e.g., loss of balance or a balance disorder), oscillopsia, or bilateral vestibulopathy.
E205. The method of any one of E201-E204, wherein the vestibular dysfunction is associated with damage to or loss of vestibular hair cells.
E206. The method of E205, wherein the damage to or loss of vestibular hair cells is associated with age (the vestibular dysfunction is age-related vestibular dysfunction), exposure to an ototoxic (e.g., vestibulotoxic) drug (the vestibular dysfunction is ototoxic drug-induced vestibular dysfunction), a disease or infection (the vestibular dysfunction is disease or infection-related vestibular dysfunction), or head trauma (the vestibular dysfunction is head trauma-related vestibular dysfunction).
E207. The method E206, wherein the ototoxic drug is an aminoglycoside (an aminoglycoside antibiotic, e.g., gentamycin, neomycin, streptomycin, tobramycin, kanamycin, vancomycin, amikacin, dibekacin, and netilmicin), viomycin, an antineoplastic drug (e.g., a platinum-containing chemotherapeutic agents, such as cisplatin, carboplatin, or oxaliplatin, or another chemotherapeutic agent, such as a nitrogen mustard or vincristine), a loop diuretic (e.g., ethacrynic acid or furosemide), a salicylate, or quinine.
E208. The method of any one of E181-E187, wherein the method further comprises evaluating the hearing of the subject prior to administering the nucleic acid vector or composition.
E209. The method of any one of E181-E187 and E208, wherein the method further comprises evaluating the hearing of the subject after administering the nucleic acid vector or composition.
E210. The method of any one of E188-E207, wherein the method further comprises evaluating the vestibular function of the subject prior to administering the nucleic acid vector or composition.
E211. The method of any one of E188-E207 and E210, wherein the method further comprises evaluating the vestibular function of the subject after administering the nucleic acid vector or composition.
E212. The method of any one of E173-E211, wherein the nucleic acid vector or composition is locally administered.
E213. The method of E212, wherein the nucleic acid vector or composition is administered to the inner ear.
E214. The method of E212, wherein the nucleic acid vector or composition is administered to the middle ear.
E215. The method of E212, wherein the nucleic acid vector or composition is administered transtympanically or intratympanically.
E216. The method of E212, wherein the nucleic acid vector or composition is administered into the perilymph.
E217. The method of E212, wherein the nucleic acid vector or composition is administered into the endolymph.
E218. The method of E212, wherein the nucleic acid vector or composition is administered to or through the oval window.
E219. The method of E212, wherein the nucleic acid vector or composition is administered to or through the round window.
E220. The method of E212, wherein the nucleic acid vector or composition is administered to a semicircular canal.
E221. The method of any one of E173-E220, wherein the nucleic acid vector or composition is administered in an amount sufficient to prevent or reduce hearing loss, delay the development of hearing loss, slow the progression of hearing loss, improve hearing, increase or induce expression of an expression product in SLC26A4-expressing cells, reduce tinnitus, improve vestibular function, reduce vertigo, improve balance, increase vestibular hair cell numbers, inhibit or slow the progression of vestibular dysfunction, reduce the feeling of fullness in the ear, increase vestibular hair cell regeneration, induce or increase the differentiation of vestibular supporting cells into vestibular hair cells, increase or induce hair cell maturation (e.g., the maturation of regenerated hair cells), or improve vestibular supporting cell function.
E222. The method of any one of E173-E221, wherein the subject is a human subject.
E223. A kit comprising the polynucleotide of any one of E1-E46 or E126-E159, the nucleic acid vector of any one of E47-E125 or E160-E165, or the composition of E166.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. Other embodiments are in the claims.

1. A polynucleotide comprising an enhancer having at least 85% sequence identity to a nucleotide sequence of SEQ ID NO: 2 or SEQ ID NO: 3 operably linked to a promoter, wherein the distance between the enhancer and the promoter in the polynucleotide is less than 3 kilobases (3kb).

2. The polynucleotide of claim 1, wherein the distance between the enhancer and the promoter is less than 500 bases.

3. The polynucleotide of claim 1, wherein the distance between the enhancer and the promoter is less than 100 bases.

4. The polynucleotide of claim 1, wherein the enhancer is directly fused to the promoter

5. The polynucleotide of any one of claims 1-4, wherein the enhancer is located 5′ of the promoter.

6. The polynucleotide of any one of claims 1-5, wherein the enhancer has the sequence of SEQ ID NO: 2 or SEQ ID NO: 3.

7. The polynucleotide of any one of claims 1-6, wherein the promoter is a constitutive promoter, a core promoter, or a minimal promoter.

8. The polynucleotide of any one of claims 1-6, wherein the promoter is a mammalian SLC26A4 promoter.

9. The polynucleotide of claim 8, wherein the promoter is a murine or human SLC26A4 promoter.

10. The polynucleotide of claim 9, wherein the murine SLC26A4 promoter has at least 85% sequence identity to the sequence of SEQ ID NO: 1.

11. The polynucleotide of claim 10, wherein the murine SLC26A4 promoter has the sequence of SEQ ID NO: 1.

12. The polynucleotide of claim 9, wherein the murine SLC26A4 promoter has at least 85% sequence identity to the sequence of SEQ ID NO: 17.

13. The polynucleotide of claim 12, wherein the murine SLC26A4 promoter has the sequence of SEQ ID NO: 17.

14. The polynucleotide of any one of claims 1-13, wherein the promoter is operably linked to a polynucleotide that can be transcribed to produce an expression product.

15. The polynucleotide of claim 14, wherein the expression product is a heterologous expression product.

16. The polynucleotide of claim 14, wherein the expression product is an expression product that is endogenously expressed in a SLC26A4-expressing inner ear cell.

17. The polynucleotide of claim 16, wherein the SLC26A4-expressing inner ear cell is an interdental cell, spiral prominence cell, cochlear root cell, or vestibular supporting cell.

18. The polynucleotide of claim 14, wherein the expression product is a mammalian pendrin protein.

19. The polynucleotide of claim 18, wherein the mammalian pendrin protein is a wild-type isoform endogenously expressed in an ear of a mammal.

20. The polynucleotide of claim 19, wherein the mammalian pendrin protein has an amino sequence of SEQ ID NO: 4 or SEQ ID NO: 5.

21. The polynucleotide of any one of claims 1-20, wherein the polynucleotide comprises an enhancer having at least 85% sequence identity to SEQ ID NO: 2 and an enhancer having at least 85% sequence identity to SEQ ID NO: 3.

22. The polynucleotide of claim 21, wherein the polynucleotide comprises, in 5′ to 3′ order, an enhancer having the sequence of SEQ ID NO: 2, an enhancer having the sequence of SEQ ID NO: 3, and a SLC26A4 promoter having the sequence of SEQ ID NO: 1 or SEQ ID NO: 17.

23. The polynucleotide of claim 22, wherein the SLC26A4 promoter has the sequence of SEQ ID NO: 17.

24. A nucleic acid vector comprising the polynucleotide of any one of claims 1-23.

25. The nucleic acid vector of claim 24, wherein the nucleic acid vector is a viral vector.

26. The nucleic acid vector of claim 25, wherein the viral vector is an adeno-associated virus vector.

27. The nucleic acid vector of any one of claims 24-26, wherein the expression product is a wild-type mammalian pendrin protein.

28. The nucleic acid vector of any one of claims 24-26, wherein the expression product is a wild-type mammalian Atoh1 protein.

29. A composition comprising the nucleic acid vector of any one of claims 24-28 and a pharmaceutically acceptable carrier, diluent, or excipient.

30. A method of expressing an expression product in an inner ear cell, comprising contacting the inner ear cell with the nucleic acid vector of any one of claims 24-28 or the composition of claim 29.

31. The method of claim 30, wherein the contacting is in a subject.

32. A method of treating a subject having or at risk of developing pendrin-related hearing loss, the method comprising the step of administering to the subject a therapeutically effective amount of the nucleic acid vector of claim 27.

33. The method of claim 32, wherein the pendrin-related hearing loss is Pendred syndrome or DFNB4.

34. A method of treating hearing loss associated with Meniere's disease in a subject in need thereof, the method comprising the step of administering to the subject a therapeutically effective amount of the nucleic acid vector of claim 27.

35. A method of treating a subject having or at risk of developing pendrin-related vestibular dysfunction, the method comprising the step of administering to the subject a therapeutically effective amount of the nucleic acid vector of claim 27.

36. A method of treating vestibular dysfunction associated with Meniere's disease in a subject in need thereof, the method comprising the step of administering to the subject a therapeutically effective amount of the nucleic acid vector of claim 27.

37. A method of treating a subject having or at risk of developing vestibular dysfunction associated with damage to or loss of vestibular hair cells, the method comprising the step of administering to the subject a therapeutically effective amount of the nucleic acid vector of claim 27 or 28.
